SEQUENCE LISTING (34 sequences)

SEQ ID NO: 1 - protein, 224 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTV

SEQ ID NO: 2 - protein, 229 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRR

SEQ ID NO: 3 - protein, 247 aa, Mus musculus
IIRRRPLFYAVSLLLPSIFLMVVDIVGFCLPPDSGERVSFKITLLLGYSVFLIIVSDTLPATIGTPLIGVYFVVCMALLVISLAETIFIVRLVHKQDLQRPVPDWLRHLVLDRIAWILCLGEQPMAHRPPATFQANKTDDCSGSDLLPAMGNHCSHVGGPQDLEKTPRGRGSPLPPPREASLAVRGLLQELSSIRHFLEKRDEMREVARDWLRVGYVLDRLLFRIYLLAVLAYSITLVTLWSIWHYS

SEQ ID NO: 4 - protein, 242 aa, Homo sapiens
IIRRRPLFYVVSLLLPSIFLMVMDIVGFYLPPNSGERVSFKITLLLGYSVFLIIVSDTLPATAIGTPLIGVYFVVCMALLVISLAETIFIVRLVHKQDLQQPVPAWLRHLVLERIAWLLCLREQSTSQRPPATSQATKTDDCSAMGNHCSHMGGPQDFEKSPRDRCSPPPPPREASLAVCGLLQELSSIRQFLEKRDEIREVARDWLRVGSVLDKLLFHIYLLAVLAYSITLVMLWSIWQYA

SEQ ID NO: 5 - protein, 202 aa, Homo sapiens
MGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 6 - protein, 471 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVIIRRRPLFYAVSLLLPSIFLMVVDIVGFCLPPDSGERVSFKITLLLGYSVFLIIVSDTLPATIGTPLIGVYFVVCMALLVISLAETIFIVRLVHKQDLQRPVPDWLRHLVLDRIAWILCLGEQPMAHRPPATFQANKTDDCSGSDLLPAMGNHCSHVGGPQDLEKTPRGRGSPLPPPREASLAVRGLLQELSSIRHFLEKRDEMREVARDWLRVGYVLDRLLFRIYLLAVLAYSITLVTLWSIWHYS

SEQ ID NO: 7 - protein, 431 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 8 - protein, 466 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVIIRRRPLFYVVSLLLPSIFLMVMDIVGFYLPPNSGERVSFKITLLLGYSVFLIIVSDTLPATAIGTPLIGVYFVVCMALLVISLAETIFIVRLVHKQDLQQPVPAWLRHLVLERIAWLLCLREQSTSQRPPATSQATKTDDCSAMGNHCSHMGGPQDFEKSPRDRCSPPPPPREASLAVCGLLQELSSIRQFLEKRDEIREVARDWLRVGSVLDKLLFHIYLLAVLAYSITLVMLWSIWQYA

SEQ ID NO: 9 - protein, 195 aa, Homo sapiens
LLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 10 - protein, 428 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYLLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 11 - protein, 233 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYY

SEQ ID NO: 12 - protein, 502 aa, Rattus norvegicus
MGGGRGGIWLALAAALLHVSLQGEFQRRLYKELVKNYNPLERPVANDSQPLTVYFSLSLLQIMDVDEKNQVLTTNIWLQMSWTDHYLQWNMSEYPGVKNVRFPDGQIWKPDILLYNSADERFDATFHTNVLVNASGHCQYLPPGIFKSSCYIDVRWFPFDVQQCKLKFGSWSYGGWSLDLQMQEADISSYIPNGEWDLMGIPGKRNEKFYECCKEPYPDVTYTVTMRRRTLYYGLNLLIPCVLISALALLVFLLPADSGEKISLGITVLLSLTVFMLLVAEIMPATSDSVPLIAQYFASTMIIVGLSVVVTVIVLRYHHHDPDGGKMPKWTRIILLNWCAWFLRMKRPGEDKVRPACQHKPRRCSLASVELSAGAGPPTSNGNLLYIGFRGLEGMHCAPTPDSGVVCGRLACSPTHDEHLMHGAHPSDGDPDLAKILEEVRYIANRNRCQDESEVICSEWKFAACVVDPLCLMAFSVFTIICTIGILMSAPNFVEAVSKDFA

SEQ ID NO: 13 - protein, 438 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKEDEAGEGRFNFSAYGMGPACLQAKDGISVKGANNSNFCYENEVTTNPPPAPSKSPEEMRKLFIQRAKKIDKISRIGFPMAFLIFNMFYWIIYKIVRREDVHNQ

SEQ ID NO: 14 - protein, 437 aa, synthetic construct
MRRAPSLVLFFLVALCGRGNCGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKEDEAGEGRFNFSAYGMGPACLQAKDGISVKGANNSNFCYENEVTTNPPPAPSKSPEEMRKLFIQRAKKIDKISRIGFPMAFLIFNMFYWIIYKIVRREDVHNQ

SEQ ID NO: 15 - protein, 496 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKEDEAGEGRFNFSAYGMGPACLQAKDGISVKGANNSNQSQPILNTKEMAPQSKPPEELEMSSMPSPVAPLPARTEGVIDMRSMSSIDSFISCATDFPEATRFTTNPPPAPSKSPEEMRKLFIQRAKKIDKISRIGFPMAFLIFNMFYWIIYKIVRREDVHNQ

SEQ ID NO: 16 - protein, 7 aa, synthetic construct
FCYENEV

SEQ ID NO: 17 - protein, 21 aa, synthetic construct
MRRAPSLVLFFLVALCGRGNC

SEQ ID NO: 18 - protein, 65 aa, synthetic construct
QSQPILNTKEMAPQSKPPEELEMSSMPSPVAPLPARTEGVIDMRSMSSIDSFISCATDFPEATRF

SEQ ID NO: 19 - DNA, 1509 bp, Rattus norvegicus
  source 1..1509: mol_type = genomic DNA
atgggcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaataactgtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcaaccgctgccaggacgagagtgaggtgatctgcagtgaatggaagtttgcagcctgcgtggtggacccgctttgcctcatggccttttcggtctttaccatcatctgtaccatcggcatcctcatgtcagctccaaactttgtggaggctgtgtccaaagactttgcttaa

SEQ ID NO: 20 - DNA, 672 bp, Homo sapiens
  source 1..672: mol_type = genomic DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaggaaccctaccctgacgtaacattcacagtt

SEQ ID NO: 21 - DNA, 687 bp, Homo sapiens
  source 1..687: mol_type = genomic DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagccctacccagatgtcaccttcacagtgaccatgcggagacgc

SEQ ID NO: 22 - DNA, 699 bp, Homo sapiens
  source 1..699: mol_type = genomic DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagccctacccagatgtcaccttcacagtgaccatgcggagacgcacactgtattac

SEQ ID NO: 23 - DNA, 744 bp, Mus musculus
  source 1..744: mol_type = genomic DNA
atcatcagaagaaggccattgttctacgccgttagtttgttgctccccagtatttttctcatggtcgtggacatcgtgggattttgtctcccacctgatagcggggagagggtctcctttaagattaccttgttgctcggctattctgtatttctgatcatcgtgtccgatacccttcctgccacaatcggcactccgctgataggagtgtatttcgtcgtgtgtatggcactcctggtgataagtctggcggaaactatcttcattgtacggctggtacataagcaggacctgcaaagacccgtgccagactggttgcgacaccttgtgctggacagaattgcatggattctgtgtcttggcgagcaacctatggcccaccggccacctgcaacctttcaagccaacaagacagacgattgtagtgggtctgatctgttgcctgctatggggaatcactgctcccatgttgggggaccacaagatttggaaaagaccccacgggggcggggatcaccccttcctcctccccgagaagcctctctcgctgtccgggggctgctccaggaactgtcaagcatccgacattttctggagaagcgggacgagatgagggaagtcgctagagactggctgcgagtgggctacgtccttgacaggctgctgtttcggatctacttgctggcggtgctggcttattccattactctggtgacactctggtccatatggcactacagttag

SEQ ID NO: 24 - DNA, 726 bp, Homo sapiens
  source 1..726: mol_type = genomic DNA
atcatccgtagaaggcctctgttttacgtggtgagcctgctgctgccatccatcttcctgatggtcatggacatcgtgggcttttacctgccacccaattctggcgagcgcgtgagcttcaagatcacactgctgctgggctatagcgtgtttctgatcatcgtgtccgataccctgcctgcaacagcaatcggaaccccactgatcggcgtgtatttcgtggtgtgcatggccctgctggtcatcagcctggccgagacaatctttatcgtgcggctggtgcacaagcaggacctgcagcagcctgtgccagcatggctgaggcacctggtgctggagaggatcgcatggctgctgtgcctgagagagcagtccacatctcagaggcctccagccacctctcaggccaccaagacagacgattgctctgccatgggcaatcactgtagccacatgggcggcccccaggactttgagaagtcccctcgcgatcggtgctctccacctccaccacctagggaggccagcctggccgtgtgcggcctgctgcaggagctgtcctctatccggcagttcctggagaagcgcgacgagatccgggaggtggccagagattggctgagggtgggcagcgtgctggataagctgctgtttcacatctacctgctggcagtcctggcctattctattaccctggtcatgctgtggtccatctggcagtacgcc

SEQ ID NO: 25 - DNA, 609 bp, Mus musculus
  source 1..609: mol_type = genomic DNA
atgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttacgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaa

SEQ ID NO: 26 - DNA, 588 bp, Homo sapiens
  source 1..588: mol_type = genomic DNA
cttttgcaaacttactttccagcaaccctcatggtgatgctttcatgggtgtccttttggatcgaccgccgagcggtccctgcacgggtccccctggggattacgacggtactgaccatgagcaccataatcactggagtcaatgcaagcatgcctagagtgtcttacataaaggccgtggacatctatctgtgggttagttttgtgttcgtattcctctccgtgctggagtatgcagctgtgaactatctgacaacagttcaagagcggaaagagcagaagttgagggagaagctgccatgcactagcggactgccaccgcccagaaccgctatgctcgatggtaactattccgacggcgaagttaatgacctcgataactacatgcctgaaaatggcgaaaagcccgacaggatgatggtccagctgacactggcctcagaaaggtccagtccacagagaaagtcacagcgatcctcttacgtcagcatgcgcatcgatacacatgccatcgacaaatactctcgcattatctttccggctgcttacatattgttcaaccttatctattggagcattttcagttga

SEQ ID NO: 27 - DNA, 1416 bp, synthetic construct
  source 1..1416: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaggaaccctaccctgacgtaacattcacagttatcatcagaagaaggccattgttctacgccgttagtttgttgctccccagtatttttctcatggtcgtggacatcgtgggattttgtctcccacctgatagcggggagagggtctcctttaagattaccttgttgctcggctattctgtatttctgatcatcgtgtccgatacccttcctgccacaatcggcactccgctgataggagtgtatttcgtcgtgtgtatggcactcctggtgataagtctggcggaaactatcttcattgtacggctggtacataagcaggacctgcaaagacccgtgccagactggttgcgacaccttgtgctggacagaattgcatggattctgtgtcttggcgagcaacctatggcccaccggccacctgcaacctttcaagccaacaagacagacgattgtagtgggtctgatctgttgcctgctatggggaatcactgctcccatgttgggggaccacaagatttggaaaagaccccacgggggcggggatcaccccttcctcctccccgagaagcctctctcgctgtccgggggctgctccaggaactgtcaagcatccgacattttctggagaagcgggacgagatgagggaagtcgctagagactggctgcgagtgggctacgtccttgacaggctgctgtttcggatctacttgctggcggtgctggcttattccattactctggtgacactctggtccatatggcactacagttag

SEQ ID NO: 28 - DNA, 1296 bp, synthetic construct
  source 1..1296: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagccctacccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttacgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaa

SEQ ID NO: 29 - DNA, 1287 bp, synthetic construct
  source 1..1287: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgttggtcaattcttcaggccactgccagtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagccctacccagatgtcaccttcacagtgaccatgcggagacgcacactgtattaccttttgcaaacttactttccagcaaccctcatggtgatgctttcatgggtgtccttttggatcgaccgccgagcggtccctgcacgggtccccctggggattacgacggtactgaccatgagcaccataatcactggagtcaatgcaagcatgcctagagtgtcttacataaaggccgtggacatctatctgtgggttagttttgtgttcgtattcctctccgtgctggagtatgcagctgtgaactatctgacaacagttcaagagcggaaagagcagaagttgagggagaagctgccatgcactagcggactgccaccgcccagaaccgctatgctcgatggtaactattccgacggcgaagttaatgacctcgataactacatgcctgaaaatggcgaaaagcccgacaggatgatggtccagctgacactggcctcagaaaggtccagtccacagagaaagtcacagcgatcctcttacgtcagcatgcgcatcgatacacatgccatcgacaaatactctcgcattatctttccggctgcttacatattgttcaaccttatctattggagcattttcagttga

SEQ ID NO: 30 - DNA, 21 bp, synthetic construct
  source 1..21: mol_type = other DNA
ttttgctatgaaaacgaagtc

SEQ ID NO: 31 - DNA, 63 bp, synthetic construct
  source 1..63: mol_type = other DNA
atgagaagggccccatccctggtattgttttttttggtagctttgtgcgggagggggaactgc

SEQ ID NO: 32 - DNA, 195 bp, synthetic construct
  source 1..195: mol_type = other DNA
caaagccaacctatccttaacactaaagagagcgccgctcaatccaaacccaaagaagagttggaaatggagtctataccttcacctgttgcacctctccctactaggaccgaaggcgtgattgacatgcgctctatgtctagtatagatagctttatatcctgcgccacagactttcccgaagccactaggttc

SEQ ID NO: 33 - DNA, 7066 bp, synthetic construct
  source 1..7066: mol_type = other DNA
agcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatccgagctccagtgtgctggaattcgcccttttcagtatttaaattgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccgttggatcaggtaagtatcaaggttacaagacaggtttaaggagaccaatagaaactgggcttgtcgagacagagaagactcttgcgtttctgataggcacctattggtcttactgacatccactttgcctttctctccacaggtgatgatatcactagtgctagcgccaccatgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttacgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaagcggccgcaattccccccgccccccccccccccccctcaccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggagatccggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaaaccggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 34 - DNA, 8167 bp, synthetic construct
  source 1..8167: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatccaggtaagtatcaaggttacaagacaggtttaaggagaccaatagaaactgggcttgtcgagacagagaagactcttgcgtttctgataggcacctattggtcttactgacatccactttgcctttctctccacaggtgatgaattcgctagcgccaccatgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttacgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaagcggccgcaattccccccgccccccccccccccccctcaccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggagatccggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaaaccggtgtcgacaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccggaactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2026/0098076 A1
(43) Pub. Date: Apr. 9, 2026

(54) MODIFIED LIGAND-GATED ION CHANNELS AND METHODS OF USE

(71) Applicant: Howard Hughes Medical Institute, Chevy Chase, MD (US)

(72) Inventors: Scott Sternson, Chevy Chase, MD (US); Peter Lee, Chevy Chase, MD (US); Christopher Magnus, Chevy Chase, MD (US)

(21) Appl. No.: 19/061,239

(22) Filed: Feb. 24, 2025

Related U.S. Application Data
(63) Continuation of application No. 17/146,906, filed on Jan. 12, 2021, now Pat. No. 12,247,064.
Application No. 17/146,906 is a continuation of application No. 16/186,122, filed on Nov. 9, 2018, now Pat. No. 10,961,296.
(60) Provisional application No. 62/584,428, filed on Nov. 10, 2017.
(60) Provisional application No. 62/729,716, filed on Sep. 11, 2018.

(51) Int. Cl. C07K 14/705; A61K 48/00

(52) U.S. Cl. CPC C07K 14/70571; A61K 48/0008; A61K 48/0058; A61K 48/0066; A61K 48/0091; C07K 2319/03

(57) ABSTRACT

This document relates to materials and methods for modulating ligand gated ion channel (LGIC) activity. For example, modified LGICs including at least one LGIC subunit having a modified ligand binding domain (LBD) and/or a modified ion pore domain (IPD) are provided. Also provided are exogenous LGIC ligands that can bind to and activate the modified LGIC, as well as methods of modulating ion transport across the membrane of a cell of a mammal, methods of modulating the excitability of a cell in a mammal, and methods of treating a mammal having a channelopathy.

### CROSS-REFERENCE TO RELATED APPLICATIONS

This application is a Continuation of U.S. application Ser. No. 17/146,906 filed Jan. 12, 2021, now allowed, which is a Continuation of U.S. application Ser. No. 16/186,122 filed Nov. 9, 2018, now U.S. Pat. No. 10,961,296, which claims the benefit of U.S. Application No. 62/584,428 filed on Nov. 10, 2017, and U.S. Application No. 62/729,716 filed on Sep. 11, 2018. The disclosures of the prior applications are considered part of (and are incorporated by reference in) the disclosure of this application.

### INCORPORATION BY REFERENCE

This application contains a Sequence Listing that has been submitted electronically as an XML file named “30872-0024003 SL_ST26.XML.” The XML file, created on Feb. 22, 2025, is 61,246 bytes in size. The material in the XML file is hereby incorporated by reference in its entirety.

### BACKGROUND

### 1. Technical Field

This document relates to materials and methods for modulating ligand gated ion channel (LGIC) activity. For example, this document provides modified LGICs including at least one LGIC subunit having a modified ligand binding domain (LBD) and/or a modified ion pore domain (IPD). Also provided are exogenous LGIC ligands that can bind to and activate the modified LGIC. In some cases, a modified LGIC and an exogenous ligand can be used to treat a mammal having a channelopathy (e.g., a neural channelopathy or a muscle channelopathy). In some cases, a modified LGIC and an exogenous LGIC ligand can be used to modulate (e.g., activate or inhibit) ion transport across the membrane of a cell of a mammal. In some cases, a modified LGIC and an exogenous LGIC ligand can be used to modulate (e.g., increase or decrease) the excitability of a cell in a mammal.

### 2. Background Information

Ion channels mediate ionic flux in cells, which profoundly affects their biological function. A prominent instance of this is in neurons, where ion channels control electrical signaling within and/or between neurons to influence physiology, sensation, behavior, mood, and cognition.

Different LGICs have distinct ligand binding properties as well as specific ion conductance properties (Hille 2001 Ion Channels of Excitable Membranes. pp. 814. Sunderland, MA: Sinauer Associates; Kandel et al 2000 Principles of Neural Science. USA: McGraw-Hill Co. 1414 pp). For example, nicotinic acetylcholine receptors (nAChRs) bind the endogenous ligand acetylcholine (ACh), which activates conductances for cations and typically depolarizes cells, thereby increasing cellular excitability. In contrast, the glycine receptor (GlyR) binds the endogenous ligand glycine, which activates chloride anion conductance and typically reduces the excitability of cells by hyperpolarization and/or by an electrical shunt of the cellular membrane resistance.

### SUMMARY

Levels of endogenous LGICligands (e.g., agonists) such as ACh are not readily controlled.

This document provides materials and methods for modulating LGIC activity (e.g., increasing the sensitivity of LGICs to exogenous ligands and/or reducing sensitivity to endogenous ligands such as ACh. For example, this document provides modified LGICs including at least one modified LGIC subunit having a LBD and an IPD, and having at least one modified amino acid (e.g., an amino acid substitution). Also provided are exogenous LGIC ligands that can bind to and modulate (e.g., activate) the modified LGIC. In some cases, a modified LGIC and an exogenous ligand can be used to treat a mammal having a channelopathy (e.g., a neural channelopathy or a muscle channelopathy). In some cases, a modified LGIC and an exogenous LGIC ligand can be used to modulate (e.g., activate or inhibit) ion transport across the membrane of a cell of a mammal. In some cases, a modified LGIC and an exogenous LGIC ligand can be used to modulate (e.g., increase or decrease) the excitability of a cell in a mammal.

Having the ability to control LGIC activity provides a unique and unrealized opportunity to control ion transport in cells. For example, modified LGICs having increased sensitivity for one or more exogenous LGIC ligands can be used to provide temporal and spatial control of ion transport and/or cellular excitability based on delivery of the exogenous LGIC ligand. For example, modified LGICs with reduced sensitivity for endogenous LGIC ligands prevent unwanted activation of modified LGICs and allow for selective control over the modified LGIC by exogenous ligands. Further, exogenous LGIC ligands having increased potency for a modified LGIC improve selectivity of targeting of the modified LGIC over endogenous ion channels. Thus, the modified LCIGs and exogenous LGIC ligands provided herein are useful to achieve a therapeutic effect while reducing side effects from the small molecules on unintended targets.

As described herein, one or more mutations in a modified LGIC can enhance potency for exogenous LGIC ligands. Mutation of the α7 LBD of α7-GlyR at residue L131 (e.g., substituting Leu with Gly or Ala) increased potency for varenicline (16-fold) and tropisetron (3.6-fold) while reducing ACh potency (−6.4-fold) relative to α7-GlyR. Mutation of α7 LBD of α7-GlyR at residue G175 (e.g., G175K) or P216 (e.g., P216I) enhanced potency for ACh, nicotine, tropisetron, varenicline, as well as other quinuclidine and tropane agonists. Combining the mutation at residue G175K with mutations that reduce potency of the endogenous agonist ACh (e.g. Y115F) produced α7-GlyR Y115F G175K with increased potency for tropisetron (5.5-fold) and reduced potency from ACh (−8-fold). In addition, combining mutations in the α7 LBD at residues 77 (e.g., substituting Trp with Phe or Tyr) and/or 79 (e.g., substituting Gln with Gly, Ala, or Ser) and/or 131 (e.g., substituting Leu with Gly or Ala) and/or 141 (e.g., substituting Leu with Phe or Pro) in these chimeric channels with potency enhancing mutations at residues G175 (e.g., G175K) or P216 (e.g., P216I) increase potency for distinct ligands and/or reduce ACh potency. For example, a chimeric α7-GlyR LGIC with a α7 nAChR LBD (α7 LBD) having a mutation at residue 79 (e.g., substituting Gln with Gly), a mutation at residue 115 (e.g., substituting Tyr with Phe), and a mutation at residue 175 (e.g., substituting Gly with Lys) has greater than 100-fold increased sensitivity to an exogenous tropane LGIC ligand compound 723 (a tropane), and reduced ACh sensitivity (−15-fold) relative to the unmodified chimeric α7-GlyR LGIC. Furthermore, a modified LGIC including at least one chimeric LGIC subunit having an α7 nAChR LBD (α7 LBD) having a mutation at residue 79 (e.g., substituting Gln with Ala, Gly, or Ser) and a GlyR IPD having a mutation at residue 298 (e.g., substituting Ala with Gly) has nearly 20-fold increased sensitivity for an exogenous LGIC ligand, such as a quinuclidine or a tropane. Additional mutations at residue 27 (e.g., substituting Arg with Asp) and 41 (e.g., substituting Glu with Arg) of the α7 LBD reduced the association of the modified chimeric LGIC with an unmodified ion channel. Additional mutations at residue 115 (e.g., substituting Tyr with Phe), 139 (e.g., substituting Gln with Gly or Leu), 210 (e.g., substituting Tyr with Phe) 217 (e.g., substituting Tyr with Phe), and/or 219 (e.g., substituting Asp with Ala) of the α7 LBD reduced sensitivity of the chimeric LGIC to the endogenous ligand ACh. These chimeric LGICs allow for highly selective control over cellular function in cells of a mammal while minimizing cross-reactivity with endogenous signaling systems in the mammal.

In general, one aspect of this document features a modified LGIC having at least one modified LGIC subunit which includes a LBD having an amino acid modification, and an IPD, where an exogenous LGIC ligand activates the modified LGIC. The modified LGIC can be a chimeric LGIC having a LBD from a first LGIC and an IPD from a second LGIC. The LBD can be an alpha7 nicotinic acetylcholine receptor (α7-nAChR) LBD. The modified LGIC, wherein the at least one modified amino acid in the α7-nAChR LBD comprises an amino acid substitution at an amino acid residue selected from the group consisting of residues 77, 79, 131, 139, 141, 175, and 216 of the α7-nAChR LBD. The amino acid substitution can be at residue 79 of the α7 LBD, and the amino acid substitution can be Q79A, Q79G, or Q79S. For example, the amino acid substitution at residue 79 of the α7 LBD can be Q79G. The IPD can be a serotonin 3 receptor (5HT3) IPD, a glycine receptor (GlyR) IPD, a gamma-aminobutyric acid (GABA) receptor IPD, or an α7-nAChR IPD. The IPD can be a GlyR IPD, and the GlyR IPD can include an amino acid substitution at residue 298 (e.g., a A298G substitution) of the chimeric LGIC. The IPD can be a GABA IPD, and the GABA IPD can include an amino acid substitution at residue 298 (e.g., a W298A substitution) of the modified LGIC. The modified LGIC can be a chimeric LGIC including an α7 LBD having a Q79G amino acid substitution, and a GlyR IPD having a A298G amino acid substitution. The exogenous LGIC ligand can be a synthetic exogenous LGIC ligand selected from the group consisting of a quinuclidine, a tropane, a 9-azabicyclo[3.3.1]nonane, a 6,7,8,9-tetrahydro-6,10-methano-6H-pyrazino (2,3-h)benzazepine, and a 1,4-diazabicyclo[3.2.2]nonane. When the synthetic exogenous LGIC ligand is a tropane, the tropane can be tropisetron, pseudo-tropisetron, nortropisetron, compound 723, compound 725, compound 737, or compound 745. When the synthetic exogenous LGIC ligand is a quinuclidine, the quinuclidine can be PNU-282987, PHA-543613, compound 0456, compound 0434, compound 0436, compound 0354, compound 0353, compound 0295, compound 0296, compound 0536, compound 0676, or compound 702. When the synthetic exogenous LGIC ligand is a 6,7,8,9-tetrahydro-6,10-methano-6H-pyrazino (2,3-h)benzazepine, the ligand can be compound 765 or compound 770. When the synthetic exogenous LGIC ligand is a 1,4-diazabicyclo[3.2.2]nonane, the ligand can be compound 773 or compound 774. In some cases, the LBD can be an α7 LBD, and the α7 LBD can also include at least one modified amino acid that confers selective binding to another α7 LBD having the at least one modified amino acid over binding to an unmodified LGIC. The unmodified LGIC can be an endogenous LGIC (e.g., an endogenous α7-nAChR). The at least one modified amino acid in the α7 LBD that confers reduced binding to the unmodified LGIC can include an amino acid substitution at residue 27 (e.g., a R27D substitution) and/or residue 41 (e.g., an E41R substitution). In some cases, the IPD can be a 5HT3 IPD, and the 5HT3 IPD can include at least one modified amino acid that confers increased ion conductance to the modified LGIC. The at least one modified amino acid in the 5HT3 IPD that confers increased ion conductance to the modified LGIC can include an amino acid substitution at an amino acid residue at residue 425 (e.g., a R425Q substitution), 429 (e.g., a R429D substitution), and/or 433 (e.g., a R433A substitution).

In another aspect, this document features a modified LGIC having at least one modified LGIC subunit including a LBD having at least one modified amino acid, and an IPD, where the at least one modified amino acid in the LBD reduces binding with an endogenous LGIC ligand. The modified LGIC can be a chimeric LGIC having a LBD from a first LGIC and an IPD from a second LGIC. The endogenous LGIC ligand can be ACh. The modified LGIC can have an EC50 of greater than 20 μM for Ach. The at least one modified amino acid can include an amino acid substitution at residue 115, 139, 210, 217, and/or 219. When the at least one modified amino acid includes an amino acid substitution at residue 115, the amino acid substitution can be a Y115F substitution. When the at least one modified amino acid includes an amino acid substitution at residue 139, the amino acid substitution can be a Q139G or a Q139L substitution. When the at least one modified amino acid includes an amino acid substitution at residue 210, the amino acid substitution can be a Y210F substitution. When the at least one modified amino acid includes an amino acid substitution at residue 217, the amino acid substitution can be a Y217F substitution. When the at least one modified amino acid includes an amino acid substitution at residue 219, the amino acid substitution can be a D219A substitution. In some cases, a modified LGIC can include a modified α7-nAChR LBD having a L131G amino acid substitution, a Q139L amino acid substitution, and a Y217F amino acid substitution. A modified LGIC (e.g., a modified LGIC including a modified α7-nAChR LBD having a L131G amino acid substitution, a Q139L amino acid substitution, and a Y217F amino acid substitution) also can include an endoplasmic reticulum export sequence. The endoplasmic reticulum export sequence can include the amino acid sequence FCYENEV (SEQ ID NO:16). For example, a modified LGIC including a modified α7-nAChR LBD having a L131G amino acid substitution, a Q139L amino acid substitution, and a Y217F amino acid substitution, and also including an endoplasmic reticulum export sequence can include the amino acid sequence set forth in SEQ ID NO: 13. A modified LGIC (e.g., a modified LGIC including a modified α7-nAChR LBD having a L131G amino acid substitution, a Q139L amino acid substitution, and a Y217F amino acid substitution) also can include a signal sequence (e.g., a CHRNB4 signal sequence). The CHRNB4 signal sequence can include the amino acid sequence MRRAPSLVLFFLVALCGRGNC (SEQ ID NO:17). For example, a modified LGIC including a modified α7-nAChR LBD having a L131G amino acid substitution, a Q139L amino acid substitution, and a Y217F amino acid substitution, and also including a CHRNB4 signal sequence can include the amino acid sequence set forth in SEQ ID NO:14. A modified LGIC (e.g., a modified LGIC including a modified α7-nAChR LBD having a L131G amino acid substitution, a Q139L amino acid substitution, and a Y217F amino acid substitution) also can include a somatic targeting sequence (e.g., a KCNB1 somatic targeting sequence). The KCNB1 somatic targeting sequence can include the amino acid sequence QSQPILNTKEMAPQSKPPEELEMSSMPSPVAPLPARTEGVIDMRSMSSIDSFISCATDFP EATRF (SEQ ID NO:18). For example, a modified LGIC including a modified α7-nAChR LBD having a L131G amino acid substitution, a Q139L amino acid substitution, and a Y217F amino acid substitution, and also including a KCNB1 somatic targeting sequence can include the amino acid sequence set forth in SEQ ID NO:15.

In another aspect, this document features a ligand having increased potency for a modified ligand gated ion channel (LGIC), wherein the ligand comprises Formula I:

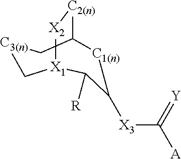

where each of X1, X2, and X3 can independently be CH, CH2, O, NH, or NMe; where each n can independently be 0 or 1; where Y=O or S; where A=an aromatic substituent; and where R=H or pyridinylmethylene. The aromatic substituent can be 1H-indole, 4-(trifluoromethyl)benzene, 2,5-dimethoxy benzene, 4-chloroaniline, aniline, 5-(trifluoromethyl)pyridin-2-yl, 6-(trifluoromethyl) nicotinic, or 4-chloro-benzene.

In some cases, a LGIC ligand can be a quinuclidine and can have a structure shown in Formula II:

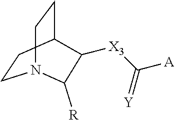

where X3=O, NH, or CH2; where Y=O or S; where A=an aromatic substituent; and where R=H or pyridinylmethylene. The aromatic substituent can be 1H-indole, 4-(trifluoromethyl)benzene, 4-chloro benzene, 2,5-dimethoxy benzene, 4-(trifluoromethyl)benzene, 4-chloroaniline, aniline, 5-(trifluoromethyl)pyridin-2-yl, 6-(trifluoromethyl) nicotinic, 3-chloro-4-fluoro benzene, or 1H-indole. The quinuclidine can be PNU-282987, PHA-543613, compound 0456, compound 0434, compound 0436, compound 0354, compound 0353, compound 0295, compound 0296, compound 0536, compound 0676, or compound 702.

In some cases, a LGIC ligand can be a tropane and can have a structure shown in Formula III:

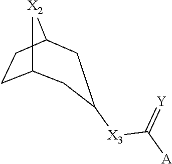

where X2=NH or NMe; where X3=O, NH, or CH2; where Y=O or S; and where A=an aromatic substituent. The aromatic substituent can be 1H-indole, 7-methoxy-1H-indole, 7-methyl-1H-indole, 5-chloro-1H-indole, or 1H-indazole. The tropane can be tropisetron, pseudo-tropisetron, nortropisetron, compound 723, compound 725, compound 737, or compound 745.

In some cases, a LGIC ligand can be a 9-azabicyclo[3.3.1]nonane and can have a structure shown in Formula IV:

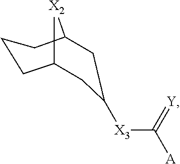

where X1 can be CH, X2 can be NH or NMe, X3 can be O, NH, or CH; Y can be O or S, and A can be an aromatic substituent. The aromatic substituent can be 4-chloro-benzene. The 9-azabicyclo[3.3.1]nonane can be compound 0536.

In another aspect, this document features a ligand having increased potency for a modified LGIC, where the ligand can be a 6,7,8,9-tetrahydro-6,10-methano-6H-pyrazino (2,3-h)benzazepine and can have a structure shown in Formula V:

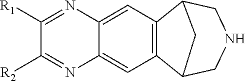

where R1 can be H, phenyl 2-toluyl; 3-pyridyl; 4-pyridyl; trifluoromethyl; methoxy; N,N-dimethylamino; N,N-diethylamino; imidazole, pyrrole, pyrazole, triazole, or isoxazole-3-amine, and where R2 can be H, methyl, or phenyl. The 6,7,8,9-tetrahydro-6,10-methano-6H-pyrazino (2,3-h)benzazepine can be varenicline, compound 0765, compound 0770, compound 0780, compound 0782, compound 0785, compound 0788, compound 0782, compound 0789, compound 0791, compound 0793, compound 0794, compound 0795, compound 0798, compound 0799, compound 0800, compound 0801, compound 0802, compound 0803, compound 0804, compound 0805, compound 0807, compound 0808, compound 0812, compound 0813, compound 815, and compound 817.

In another aspect, this document features a ligand having increased potency for a modified LGIC, where the ligand can be a 2-(pyridin-3-yl)-1,5,6,7,8,9-hexahydro-5,9-methanoimidazo[4′,5′: 4,5]benzo[1,2-d]azepine (e.g., compound 0786):

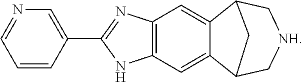 [SMILES: c1cncc(-c2nc3cc4c(cc3[nH]2)C2CNCC4C2)c1]

In another aspect, this document features a ligand having increased potency for a modified LGIC, where the ligand can be 7,8,9,10-tetrahydro-1H-6,10-methanoazepino[4,5-g]quinoxalin-2 (6H)-one and can have a structure shown in Formula VI:

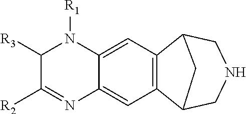

where R1 can be H or CH₃, where R2 can be H, CH3, or an aromatic substituent, and where R3 can be O or S. The 7,8,9,10-tetrahydro-1H-6,10-methanoazepino[4,5-g]quinoxalin-2 (6H)-one can be compound 0783, compound 0784, compound 0790, or compound 0792.

In another aspect, this document features a ligand having increased potency for a modified LGIC, where the ligand can be a 1,4-diazabicyclo[3.2.2]nonane and can have a structure shown in Formula VII:

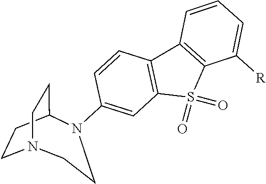

where R can be H, F, or NO₂. The 1,4-diazabicyclo[3.2.2]nonane can be 3-(1,4-diazabicyclo[3.2.2]nonan-4-yl)dibenzo[b,d]thiophene 5,5-dioxide, compound 0773, or compound 0774.

In another aspect, this document features methods of treating a channelopathy in a mammal. The methods include, or consist essentially of, administering to a cell in the mammal a modified LGIC, where an exogenous LGIC ligand selectively binds the modified LGIC. The modified LGIC has at least one modified LGIC subunit including a LBD including at least one modified amino acid, and an IPD; and administering the exogenous ligand to the mammal. The channelopathy can be Bartter syndrome, Brugada syndrome, catecholaminergic polymorphic ventricular tachycardia (CPVT), congenital hyperinsulinism, cystic fibrosis, Dravet syndrome, episodic ataxia, erythromelalgia, generalized epilepsy (e.g., with febrile seizures), familial hemiplegic migraine, fibromyalgia, hyperkalemic periodic paralysis, hypokalemic periodic paralysis, Lambert-Eaton myasthenic syndrome, long QT syndrome (e.g., Romano-Ward syndrome), short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, or multiple sclerosis.

In another aspect, this document features methods of modulating ion transport across a cell membrane of a mammal. The methods include, or consist essentially of, administering to the cell a modified LGIC, where an exogenous LGIC ligand selectively binds the modified LGIC. The modified LGIC has at least one modified LGIC subunit including a LBD including at least one modified amino acid, and an IPD; and administering the exogenous ligand to the mammal. The modulating can include activating or inhibiting ion transport. The cell can be a neuron, a glial cell, a myocyte, a stem cell, an endocrine cell, or an immune cell. The administering the modified LGIC to the cell can be an in vivo administration or an ex vivo administration. The administering the modified LGIC to the cell can include administering a nucleic acid encoding the modified LGIC.

In another aspect, this document features methods of modulating the excitability of a cell in a mammal. The methods include, or consist essentially of, administering to the cell from the mammal a modified LGIC, where an exogenous LGIC ligand selectively binds the modified LGIC. The modified LGIC has at least one modified LGIC subunit including a LBD including at least one modified amino acid, and an IPD; and administering the exogenous ligand to the mammal. The modulating can include increasing the excitability of the cell or decreasing the excitability of the cell. The cell can be an excitable cell. The cell can be a neuron, a glial cell, a myocyte, a stem cell, an endocrine cell, or an immune cell. The administering the modified LGIC to the cell can be an in vivo administration or an ex vivo administration. The administering the modified LGIC to the cell can include administering a nucleic acid encoding the modified LGIC.

In another aspect, this document features methods of modulating the activity of a cell in a mammal. The methods include, or consist essentially of, administering to the cell a modified LGIC, where an exogenous LGIC ligand selectively binds the modified LGIC. The modified LGIC has at least one modified LGIC subunit including a LBD including at least one modified amino acid, and an IPD; and administering the exogenous ligand to the mammal. The modulating can include increasing the activity of the cell or decreasing the activity of the cell. The activity can be ion transport, passive transport, excitation, inhibition, or exocytosis. The cell can be a neuron, a glial cell, a myocyte, a stem cell, an endocrine cell, or an immune cell. The administering the modified LGIC to the cell can be an in vivo administration or an ex vivo administration. The administering the modified LGIC to the cell can include administering a nucleic acid (e.g., via a viral vector such as an adeno-associated virus, a herpes simplex virus, or a lentivirus) encoding the modified LGIC.

In another aspect, this document features a method for identifying a ligand that selectively binds to a modified LGIC. The method includes, or consists essentially of, providing one or more candidate ligands to the modified LGIC described herein, and detecting binding between the candidate ligand and the modified LGIC, thereby identifying a ligand that selectively binds the modified LGIC. The modified LGIC can be a homomeric modified LGIC.

In another aspect, this document features a method for detecting a modified LGIC. The method includes, or consists essentially of, providing one or more modified LGIC subunits described herein, providing an agent that selectively binds the modified LGIC, and detecting binding between the modified LGIC and the agent that selectively binds the modified LGIC, thereby detecting the modified LGIC. The agent that selectively binds the modified LGIC comprises can be antibody, a protein (e.g., bungarotoxin), or a small molecule (e.g., a positron emission tomography (PET) ligand). The agent that selectively binds the modified LGIC can include a detectable label (e.g., a fluorescent label, a radioactive label, or a positron emitting label).

In another aspect, this document features synthetic nucleic acid constructs including a nucleic acid sequence having at least 75 percent sequence identity to the sequence set forth in SEQ ID NO:27. The nucleic acid sequence having at least 75 percent sequence identity to the sequence set forth in SEQ ID NO:27 can include a nucleic acid sequence that can encode a LBD having an amino acid modification. The modified LBD can be an α7-nAChR LBD having an amino acid substitution residue 77, 79, 115, 131, 139, 141, 175, 210, 216, 217, and/or 219 of the α7-nAChR LBD. The nucleic acid sequence having at least 75 percent sequence identity to the sequence set forth in SEQ ID NO:27 can include a nucleic acid sequence that can encode an IPD. The IPD can be a 5HT3 IPD, a GlyR IPD, a GABA receptor IPD, or an α7-nAChR IPD. The IPD can be a 5HT3 IPD.

In another aspect, this document features synthetic nucleic acid constructs including a nucleic acid sequence having at least 75 percent sequence identity to the sequence set forth in SEQ ID NO:28.

The nucleic acid sequence having at least 75 percent sequence identity to the sequence set forth in SEQ ID NO:28 can include a nucleic acid sequence that can encode a LBD having an amino acid modification. The modified LBD can be an α7-nAChR LBD having an amino acid substitution residue 77, 79, 115, 131, 139, 141, 175, 210, 216, 217, and/or 219 of the α7-nAChR LBD. The nucleic acid sequence having at least 75 percent sequence identity to the sequence set forth in SEQ ID NO:28 can include a nucleic acid sequence that can encode an IPD. The IPD can be a 5HT3 IPD, a GlyR IPD, a GABA receptor IPD, or an α7-nAChR IPD. The IPD can be a GlyR IPD having an amino acid modification. The modified GlyR IPD can have an amino acid substitution at amino acid residue 298 of the GlyR IPD.

In another aspect, this document features synthetic nucleic acid constructs including a nucleic acid sequence having at least 75 percent sequence identity to the sequence set forth in SEQ ID NO:29. The nucleic acid sequence having at least 75 percent sequence identity to the sequence set forth in SEQ ID NO:29 can include a nucleic acid sequence that can encode a LBD having an amino acid modification. The modified LBD can be a α7-nAChR LBD having an amino acid substitution residue 77, 79, 115, 131, 139, 141, 175, 210, 216, 217, and/or 219 of the α7-nAChR LBD. The nucleic acid sequence having at least 75 percent sequence identity to the sequence set forth in SEQ ID NO:29 can include a nucleic acid sequence that can encode an IPD. The IPD can be a 5HT3 IPD, a GlyR IPD, a GABA receptor IPD, or an α7-nAChR IPD. The IPD can be a GABA IPD having an amino acid modification. The modified GABA IPD can have an amino acid substitution at amino acid residue 298 of the GABA IPD.

In another aspect, this document features synthetic nucleic acid constructs having the sequence set forth in SEQ ID NO: 33.

In another aspect, this document features synthetic nucleic acid constructs having the sequence set forth in SEQ ID NO: 34.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E show exemplary amino acid sequences of chimeric LGICs. Mutation of amino acid residue 77 (e.g., W77F or W77Y) resulted in sensitivity to granisetron and tropisetron. Mutation of amino acid residue 79 (e.g., Q79G) was most effective for several agonists. Mutations of amino acid residue 131 (e.g., L131G, L131A, L131M, or L131N) altered sensitivity to varenicline, tropisetron, granisetron, and ACh. Potency was considerably enhanced when LBD mutations were combined with mutation at amino acid residue 298 in the GlyR or GABAC IPD. Potency was also enhanced when α7 nAChR LBD mutations were combined with mutation at amino acid residue G175 and P216. FIG. 1A) An amino acid sequence of α7-5HT3 chimeric receptor (SEQ ID NO:6) including a human α7 nAChR LBD (SEQ ID NO:1) and a murine 5HT3 IPD (SEQ ID NO:3) components. FIG. 1B) An amino acid sequence of α7-GlyR chimeric receptor (SEQ ID NO:7), including a human α7 nAChR LBD (SEQ ID NO:2) and a human GlyR IPD (SEQ ID NO:5) components. FIG. 1C) An amino acid sequence of α7-5HT3 chimeric receptor (SEQ ID NO: 8) including human α7 nAChR LBD (SEQ ID NO:1) and a human 5HT3 IPD (SEQ ID NO: 4) components. FIG. 1D) An amino acid sequence of α7-GABAc chimeric receptor (SEQ ID NO:10) including a human α7 nAChR LBD (SEQ ID NO:11) and a human GABAc IPD (SEQ ID NO:9) components. FIG. 1E) An amino acid sequence of rat nAChR sequence (SEQ ID NO:12).

FIG. 2 shows EC50s for tropisetron against a α7-5HT3 chimeric LGIC and variants of the chimeric LGIC with LBD mutations at positions noted in FIG. 1. Multiple mutations at Gln79 showed similar or improved potency relative to the unmodified α7-5HT3 channel (arrows).

FIGS. 3A-3B shows the relative potency of known nAChR agonists for α7-5HT3 chimeric LGICs. FIG. 3A) A graph of EC50s normalized to the unmodified α7-5HT3 chimeric channel (log scale). *P<0.05, statistically significant potency changes are noted (ANOVA followed by Dunn's test). FIG. 3B) Chemical structures of known nAChR agonists.

FIGS. 4A-4C show the relative potency of known nAChR agonists for α7-GlyR chimeric LGICs. FIG. 4A) A graph of EC50s for Q79 LBD mutants normalized to the unmodified α7-GlyR chimeric channel (log scale). FIG. 4B) A graph of EC50s for A298G IPD mutation normalized to the unmodified α7-GlyR chimeric channel (log scale). FIG. 4C) A graph of EC50s for α7-GlyR^{A298G} normalized to the unmodified α7-GlyR chimeric channel and compared to the double mutant channel α7Q79G-GlyR^{A298G} (log scale). *P<0.05, statistically significant potency changes are noted (ANOVA followed by Dunn's test).

FIGS. 5A-5C show schematic structures of LGIC agonists with substitution patterns most compatible with potency enhancement for α7^{Q79G}-5HT3 and α7^{Q79G}-GlyR^{A298G}. FIG. 5A) A generalized structure showing attributes associated with enhanced potency. FIG. 5B) Specific pharmacophores represented in (FIG. 5A) are quinuclidine, tropane, and 9-azabicyclo[3.3.1]nonane core structures. FIG. 5C) Exemplary synthetic molecules that show high potency for α7^{79G}-GlyR^{A298G}, α7^{Q79G,Y115F,G175K}-GlyR, α7^{W77F,Q79G,G175K}-GlyR.

FIGS. 6A-6C show mutations that reduce association of chimeric LCIG α7 nAChR LBDs with unmodified LBDs. FIG. 6A) Charge reversal schematic potential configurations of transfecting two epitope tagged (HA and V5) constructs encoding α7-5HT3 (top) or two constructs encoding α7-5HT3-HA and α7^{R21D,EH1R}-5HT3-V5 where association between the two different epitope tagged subunits would be unfavored due to charge reversal mutations at the subunit interfaces. FIG. 6B) Whole cell recordings in HEK cells expressing α7^{R21D,E4IR}-5HT3 with a V5 epitope tag shows potent responses to PNU-282987. FIG. 6C) Association of α7-5HT3 LGICs with HA and V5 epitope tags in HEK cells was probed by HA immunoprecipitation (left) or total lysate isolation followed by western blotting with either anti-HA (top) or anti-V5 antibodies (bottom). In cells co-expressing channels with the HA and V5 epitopes, anti-HA IP followed by anti-V5 immunoblotting shows the co-immunoprecipitation of unmodified channels of each type, but charge reversal mutations in the LBD α7^{R21D,F4IR}-5HT3-V5 was not immunoprecipitated. MW of α7-5HT3 is ˜48 kD (arrow).

FIG. 7 shows that chimeric LGICs can be controlled using an exogenous ligand. Cortical neurons from a mouse brain transduced with α779G-GlyR^{A298G} chimeric LGIC via adeno-associated virus (AAV) vectors fires action potentials in response to 40 pA current injection (PRE) that are potently suppressed by 30 nM tropisetron. After washout (WASH) of tropisetron, neuron firing is restored.

FIGS. 8A-8C show activity of agonists on chimeric LGICs with a G175K mutation. FIG. 8A) A graph of EC50s for Q79G G175K LBD mutants against known agonists normalized to the unmodified α7-GlyR chimeric channel (log scale). FIG. 8B) A graph of EC50s for ACh and tropisetron for channels with mutations in α7-GlyR chimeric LGICs. Mutations that result in channels with high potency for tropisetron and low potency for the endogenous ligand, acetylcholine (ACh) are optimal (grey shading). Unmod.: unmodified α7-GlyR chimeric LGIC. FIG. 8C) Action potentials of cortical neurons from a mouse brain transduced with α7^{Q79G,Y115F,G175K}-GlyR chimeric LGIC. Neurons fire in response to current injection (PRE) and are potently suppressed by 100 nM tropisetron. After washout (WASH) of tropisetron, neuron firing is restored.

FIGS. 9A-9E show activity of agonists on chimeric LGICs with a L131G mutation. FIG. 9A) A graph of EC50s for L131 LBD mutants against known agonists normalized to the unmodified α7-GlyR chimeric channel (log scale). FIG. 9B) A graph of EC50s for ACh and tropisetron for channels with mutations in α7^{L131G}-GlyR chimeric LGICs. FIG. 9C) A graph showing mutations that result in channels with high potency for varenicline and low potency for the endogenous ligand, acetylcholine (ACh) are optimal (grey shading). Unmod.: unmodified α7-GlyR chimeric LGIC. FIG. 9D) A graph showing activation of channels with mutations in α7L131G-GlyR by Ach and varenicline after brief channel antagonism by picrotoxin (PTX). Solid lines represent duration of molecule administration. FIG. 9E) Action potentials of a cortical neuron from a mouse brain transduced with α7^{L131G,Q139I,Y217F}-GlyR chimeric LGIC. Neuron fires in response to current injection (PRE) and are potently suppressed by 10 nM varenicline, even with >6-fold greater injected current. After washout (WASH) of varenicline, neuron firing is restored.

FIG. 10 contains a graph showing that varenicline can activate chimeric receptors in mice to induce a behavioral effect. In this case, expressing α7^{L131G,Q139I,Y217F}-GlyR in the VGAT-expressing neurons unilaterally in the substantia nigra reticulate followed by varenicline administration leads to contralateral rotation, consistent with varenicline-induced silencing the a 7^{L131G,Q139I,Y217F}-GlyR expressing neurons.

FIGS. 11A-11C show chemical structures of exemplary LGIC agonists. FIG. 11A) Chemical structures of LGIC agonists with substitution patterns most compatible with potency enhancement for α7^{Q79G,Y115F,G175K}-GlyR. FIG. 11B) Chemical structures of LGIC agonists with substitution patterns most compatible with potency enhancement for α7^{L131G,Q139L,Y217F}-GlyR, α7^{L131G,Q139I,Y217F}-5HT3, or α7^{L131G,Q139I,Y217F}-5HT3 HC. FIG. 11C) Chemical structures of LGIC agonists with substitution patterns most compatible with potency enhancement for α7^{L131G,Q139I,Y217F}-GlyR or α7^{L131G,Q139I,Y217F}-5HT3 HC.

FIGS. 12A-12F show chemogenetic perturbation of cortical neuron activity. FIG. 12A-FIG. 12B) Action potential firing was strongly suppressed by varenicline strongly in neurons expressing PSAM⁴-GlyR due to reduced input resistance (FIG. 12A) and elevated rheobase (FIG. 12B). FIG. 12C) Cortical layer 2/3 neuron membrane properties were similar in PSAM⁴-GlyR expressing neurons and intermingled untransfected control neurons. FIG. 12D-FIG. 12E) Varenicline depolarizes (FIG. 12D) and elicits firing (FIG. 12E) in neurons expressing PSAM⁴-5HT3 HC. FIG. 12F) Cortical layer 2/3 neuron membrane properties are similar in PSAM⁴-5HT3 HC expressing neurons and intermingled untransfected control neurons. Data are mean±SEM. Mann Whitney U-test, n.s. P>0.05, ***P<0.001.

FIGS. 13A-13D show PSAM⁴-GlyR neuron silencing in mice. FIG. 13A) PSAM⁴-GlyR-IRES-EGFP targeted unilaterally to the SNr. Inset: schematic of unilateral SNr transduction were silencing results in contraversive rotation. Asterisk: nonspecific immunofluorescence. FIG. 13B) Low doses of intraperitoneal varenicline elicit contraversive rotation for mice expressing PSAM⁴-GlyR but not sham operated or EGFP-alone expressing mice. FIG. 13C) Two doses of varenicline separated by 5 h give similar proportion of total rotation, indicating no tachyphylaxis of the chemogenetic response. FIG. 13D) Duration of chemogenetic silencing monitored by the timecourse of rotation response normalized to maximum rotation for each mouse. Pink (narrow) arrows: amphetamine injections, cyan (wide) arrow: varenicline injection. Mann-Whitney U-test, n.s. P>0.05, **P<0.01.

FIGS. 14A-14J show ultrapotent chemogenetic agonists (uPSEMs). FIG. 14A) Comparison of uPSEM agonist EC50s at PSAM⁴ channels and endogenous varenicline targets, as well as IC50 for α4β2 nAChR with 1 μM ACh. LED: lowest effective dose for mice in SNr rotation assay. Units: nM; parentheses: SEM. Selectivity relative to PSAM⁴-GlyR in bold. FIG. 14B, FIG. 14C) Dose response curves for PSAM⁴-GlyR, α4β2 nAChR, 5HT3-R. uPSEM⁷⁹² (FIG. 14B) is a 10% partial agonist of α4β2 nAChR and uPSEM⁸¹⁷ (FIG. 14C) inhibits α4β2 nAChR. FIG. 14D) Current response to uPSEMs (2 μM) and Ach (10 μM) in a HEK cell expressing α7 nAChRs. uPSEMs do not activate α7 nAChR, while ACh does. (FIG. 14E) Response amplitude normalized to ACh. FIG. 14F-FIG. 14J) uPSEM⁷⁹², uPSEM⁷⁹³, uPSEM⁸¹⁵, and uPSEM⁸¹⁷ strongly suppress firing in cortical neurons expressing PSAM⁴-GlyR by reducing the current required to fire an action potential (rheobase) (FIG. 14G-FIG. 14J).

FIGS. 15A-15H show in vivo uPSEM dose responses for mice expressing PSAM⁴-GlyR unilaterally in SNr. Behavioral response and timecourse for uPSEM⁷⁹² (FIG. 15A, FIG. 15B), uPSEM⁷⁹³ (FIG. 15C, FIG. 15D), uPSEM⁸¹⁵ (FIG. 15E, FIG. 15F), uPSEM⁸¹⁷ (FIG. 15G, FIG. 15H). Timecourse of rotation response normalized to maximum rotation for each mouse. Pink (narrow) arrows: amphetamine injections, blue (wide) arrows: uPSEM injection.

FIG. 16 shows an exemplary amino acid sequence for PSAM4-GlyR-KirM3M4 (SEQ ID NO:13). PSAM⁴ mutations are highlighted. A sequence for a Kir2.1 (KCNJ2) endoplasmic reticulum (ER) export sequence is underlined and in blue.

FIG. 17 shows an exemplary amino acid sequence for PSAM⁴-GlyR-B4sig (SEQ ID NO: 14). PSAM⁴ mutations are highlighted. A sequence for a β4 nAChR subunit (CHRNB4) signal sequence is underlined and in blue.

FIG. 18 shows an exemplary amino acid sequence for PSAM⁴-GlyR-Kv2M3M4-soma (SEQ ID NO:15). PSAM⁴ mutations are highlighted. Sequence for Kv2.1 (KCNB1) somatic targeting sequence underlined and in blue.

FIGS. 19A-19B show an exemplary construct including a nucleic acid sequence encoding a modified LGIC subunit. (FIG. 19A) A plasmid map of an AAV-Syn::PSAM4-GlyR-IRES-EGFP-WPRE construct including a nucleic acid sequence (SEQ ID NO:33) encoding a modified LGIC subunit. (FIG. 19B-1-FIG. 19B-3) A nucleic acid sequence of an AAV-Syn::PSAM4-GlyR-IRES-EGFP-WPRE construct including a nucleic acid sequence encoding a modified LGIC subunit.

FIGS. 20A-20B show an exemplary construct including a nucleic acid sequence encoding a modified LGIC subunit. (FIG. 20A) A plasmid map of an AAV-CamkII::PSAM4-GlyR-IRES-EGFP-WPRE construct including a nucleic acid sequence (SEQ ID NO:34) encoding a modified LGIC subunit. (FIG. 20B-1-FIG. 20B-3) A nucleic acid sequence of an AAV-CamkII::PSAM4-GlyR-IRES-EGFP-WPRE construct including a nucleic acid sequence encoding a modified LGIC subunit.

### DETAILED DESCRIPTION

This document provides modified LGICs and methods of using them. For example, this document provides modified LGICs including at least one modified LGIC subunit having a LBD and an IPD, and having at least one modified amino acid (e.g., an amino acid substitution). In some cases, a modified LGIC can be a chimeric LGIC. For example, a chimeric LGIC can include a LBD from a first LGIC and an IPD from a second LGIC. In some cases, the modified amino acid can confer pharmacological selectivity to the modified LGIC. For example, the modified amino acid can confer the modified LGIC with selective binding of an exogenous LGIC ligand. For example, the modified amino acid can confer the modified LGIC with reduced (e.g., minimized or eliminated) binding of an unmodified LGIC subunit (e.g., an LGIC subunit lacking the modification and/or an endogenous LGIC subunit). For example, the modified amino acid can confer the modified LGIC with reduced (e.g., minimized or eliminated) binding of an endogenous LGIC ligand.

Modified LGICs provided herein can be used, for example, in methods for treating channelopathies (e.g., a neural channelopathy or a muscle channelopathy). For example, a modified LGIC, and an exogenous LGIC ligand that can bind to and activate the modified LGIC, can be used to treat a mammal having a channelopathy. In some cases, a modified LGIC and an exogenous LGIC ligand can be used to modulate (e.g., activate or inhibit) ion transport across the membrane of a cell of a mammal. In some cases, a modified LGIC and an exogenous LGIC ligand can be used to modulate (e.g., increase or decrease) the excitability of a cell in a mammal.

### Modified LGICs

As used herein a “modified” LGIC is an LGIC that includes at least one LGIC subunit. Modified LGIC can also be referred to as pharmacologically selective actuator modules (PSAMs). A modified LGIC subunit can include at least one modified amino acid (e.g., an amino acid substitution) in the LBD and/or at least one modified amino acid (e.g., an amino acid substitution) in the IPD. A modified LGIC subunit described herein can be a modification of an LGIC from any appropriate species (e.g., human, rat, mouse, dog, cat, horse, cow, goat, pig, or monkey). In some cases, a modified LGIC can include at least one chimeric LGIC subunit having a non-naturally occurring combination of a LBD from a first LGIC and an IPD from a second LGIC.

A modified LGIC (e.g., an LGIC including one or more modified LGIC subunits) can be a homomeric (e.g., having any number of the same modified LGIC subunits) or heteromeric (e.g., having at least one modified LGIC subunit and any number of different LGIC subunits). In some cases, a modified LGIC described herein can be a homomeric modified LGIC. A modified LGIC described herein can include any suitable number of modified LGIC subunits. In some cases, a modified LGIC can be a trimer, a tetramer, a pentamer, or a hexamer. For example, a modified LGIC described herein can be a pentamer.

A modified LGIC subunit described herein can be a modification of any appropriate LGIC. The LGIC can conduct anions, cations, or both through a cellular membrane in response to the binding of a ligand. For example, the LGIC can transport sodium (Na+), potassium (K+), calcium (Ca2+), and/or chloride (Cl−) ions through a cellular membrane in response to the binding of a ligand. Examples of LGICs include, without limitation, Cys-loop receptors (e.g., AChR such as a nAChR (e.g., a muscle-type nAChR or a neuronal-type nAChR), gamma-aminobutyric acid (GABA; such as GABAA and GABAA-P (also referred to as GABAc) receptors, GlyR, GluCl receptors, and 5HT3 receptors), ionotropic glutamate receptors (iGluR; such as AMPA receptors, kainate receptors, NMDA receptors, and delta receptors), ATP-gated channels (e.g., P2X), and phosphatidylinositol 4,5-bisphosphate (PIP2)-gated channels. In cases where a modified LGIC described herein is a chimeric LGIC, the chimeric LGIC can include a LBD selected from any appropriate LGIC and an IPD selected from any appropriate LGIC. In cases where a LGIC includes multiple different subunits (for example, a neuronal-type nAChR includes α4, β2, and α7 subunits), the LBD and/or IPD can be selected from any of the subunits. For example, a LBD from a nAChR can be a α7 LBD. A representative rat α7 nAChR amino acid sequence (including both a LBD and an IPD) is as follows.

SEQ ID NO: 12
MGGGRGGIWLALAAALLHVSLQGEFQRRLYKELVKNYNPLERPVANDSQP
LTVYFSLSLLQIMDVDEKNQVLTTNIWLQMSWTDHYLQWNMSEYPGVKNV
RFPDGQIWKPDILLYNSADERFDATFHTNVLVNASGHCQYLPPGIFKSSC
YIDVRWFPFDVQQCKLKFGSWSYGGWSLDLQMQEADISSYIPNGEWDLMG
IPGKRNEKFYECCKEPYPDVTYTVTMRRRTLYYGLNLLIPCVLISALALL
VFLLPADSGEKISLGITVLLSLTVFMLLVAEIMPATSDSVPLIAQYFAST
MIIVGLSVVVTVIVLRYHHHDPDGGKMPKWTRIILLNWCAWFLRMKRPGE
DKVRPACQHKPRRCSLASVELSAGAGPPTSNGNLLYIGFRGLEGMHCAPT
PDSGVVCGRLACSPTHDEHLMHGAHPSDGDPDLAKILEEVRYIANRNRCQ
DESEVICSEWKFAACVVDPLCLMAFSVFTIICTIGILMSAPNFVEAVSKD
FA

In some cases, a modified LGIC subunit described herein can include a LBD from a α7 nAChR. Examples of α7 nAChR LBDs include, without limitation, a human α7 nAChR LBD having the amino acid sequence set forth in SEQ ID NO: 1, a human α7 nAChR LBD having the amino acid sequence set forth in SEQ ID NO:2, and a human α7 nAChR LBD having the amino acid sequence set forth in SEQ ID NO:11. In some cases, a α7 nAChR LBD can be a homolog, orthologue, or paralog of the human α7 nAChR LBD set forth in SEQ ID NO:1, SEQ ID NO:2, or SEQ ID NO:11. In some cases, a α7 nAChR LBD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:1, SEQ ID NO:2, or SEQ ID NO:11.

SEQ ID NO: 1
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTV
SEQ ID NO: 2
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRR
SEQ ID NO: 11
MRCSPGGVWLALAASLLHVSLQGEFQRKLYKELVKNYNPLERPVANDSQP
LTVYFSLSLLQIMDVDEKNQVLTTNIWLQMSWTDHYLQWNVSEYPGVKTV
RFPDGQIWKPDILLYNSADERFDATFHTNVLVNSSGHCQYLPPGIFKSSC
YIDVRWFPFDVQHCKLKFGSWSYGGWSLDLQMQEADISGYIPNGEWDLVG
IPGKRSERFYECCKEPYPDVTFTVTMRRRTLYY

In some cases, a modified LGIC subunit described herein can include a IPD from a 5HT3 receptor. Examples of 5HT3 IPDs include, without limitation, a murine 5HT3 IPD having the amino acid sequence set forth in SEQ ID NO:3, and a human 5HT3 IPD having the amino acid sequence set forth in SEQ ID NO:4. In some cases, a 5HT3 IPD can be a homolog, orthologue, or paralog of a 5HT3 IPD set forth in SEQ ID NO:3 or SEQ ID NO:4. In some cases, a 5HT3 IPD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:3 of SEQ ID NO:4.

SEQ ID NO: 3
IIRRRPLFYAVSLLLPSIFLMVVDIVGFCLPPDSGERVSFKITLLLGYSV
FLIIVSDTLPATIGTPLIGVYFVVCMALLVISLAETIFIVRLVHKQDLQR
PVPDWLRHLVLDRIAWILCLGEQPMAHRPPATFQANKTDDCSGSDLLPAM
GNHCSHVGGPQDLEKTPRGRGSPLPPPREASLAVRGLLQELSSIRHFLEK
RDEMREVARDWLRVGYVLDRLLFRIYLLAVLAYSITLVTLWSIWHYS
SEQ ID NO: 4
IIRRRPLFYVVSLLLPSIFLMVMDIVGFYLPPNSGERVSFKITLLLGYSV
FLIIVSDTLPATAIGTPLIGVYFVVCMALLVISLAETIFIVRLVHKQDLQ
QPVPAWLRHLVLERIAWLLCLREQSTSQRPPATSQATKTDDCSAMGNHCS
HMGGPQDFEKSPRDRCSPPPPPREASLAVCGLLQELSSIRQFLEKRDEIR
EVARDWLRVGSVLDKLLFHIYLLAVLAYSITLVMLWSIWQYA

In some cases, a modified LGIC subunit described herein can include an IPD from a GlyR. Examples of GlyR IPDs include, without limitation, a murine GlyR IPD having the amino acid sequence set forth in SEQ ID NO:5. In some cases, a GlyR IPD can be a homolog, orthologue, or paralog of the human GlyR IPD set forth in SEQ ID NO:5. In some cases, a GlyR IPD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:5.

SEQ ID NO: 5
MGYYLIQMYIPSLLIVILSWISFWINMDAAPARVGLGITTVLTMTTQSSG
SRASLPKVSYVKAIDIWMAVCLLFVFSALLEYAAVNFVSRQHKELLRFRR
KRRHHKEDEAGEGRFNFSAYGMGPACLQAKDGISVKGANNSNTTNPPPAP
SKSPEEMRKLFIQRAKKIDKISRIGFPMAFLIFNMFYWIIYKIVRREDVH
NQ

In some cases, a modified LGIC subunit described herein can include an IPD from a GABA receptor (e.g., GABAA-P, also referred to as GABAc). Examples of GABAA-P IPDs include, without limitation, a human GABAA-p IPD having the amino acid sequence set forth in SEQ ID NO:9. In some cases, a GABAA-p IPD can be a homolog, orthologue, or paralog of the human GABAA-p IPD set forth in SEQ ID NO:9. In some cases, a GABAA-P IPD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:9.

SEQ ID NO: 9
LLQTYFPATLMVMLSWVSFWIDRRAVPARVPLGITTVLTMSTIITGVNAS
MPRVSYIKAVDIYLWVSFVFVFLSVLEYAAVNYLTTVQERKEQKLREKLP
CTSGLPPPRTAMLDGNYSDGEVNDLDNYMPENGEKPDRMMVQLTLASERS
SPQRKSQRSSYVSMRIDTHAIDKYSRIIFPAAYILFNLIYWSIFS

In calculating percent sequence identity, two sequences are aligned and the number of identical matches of amino acid residues between the two sequences is determined. The number of identical matches is divided by the length of the aligned region (i.e., the number of aligned amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. The alignment of two or more sequences to determine percent sequence identity can be performed using the computer program ClustalW and default parameters, which calculates the best match between a query and one or more subject sequences, and aligns them so that identities, similarities and differences can be determined. See, e.g., Chenna et al., 2003, Nucleic Acids Res., 31 (13): 3497-500.

In cases where a modified LGIC subunit described herein is a chimeric LGIC subunit, the chimeric LGIC subunit can include a LBD and IPD from the same species or a LBD and IPD from different species. In some cases, a chimeric LGIC subunit can include a LBD from a human LGIC protein and an IPD from a human LGIC protein. For example, a chimeric LGIC subunit can include a human α7 LBD and a human GlyR IPD. In some cases, a chimeric LGIC subunit can include a LBD from a human LGIC protein and an IPD from a murine LGIC protein. For example, a chimeric LGIC subunit can include a human α7 LBD and a murine 5HT3 IPD.

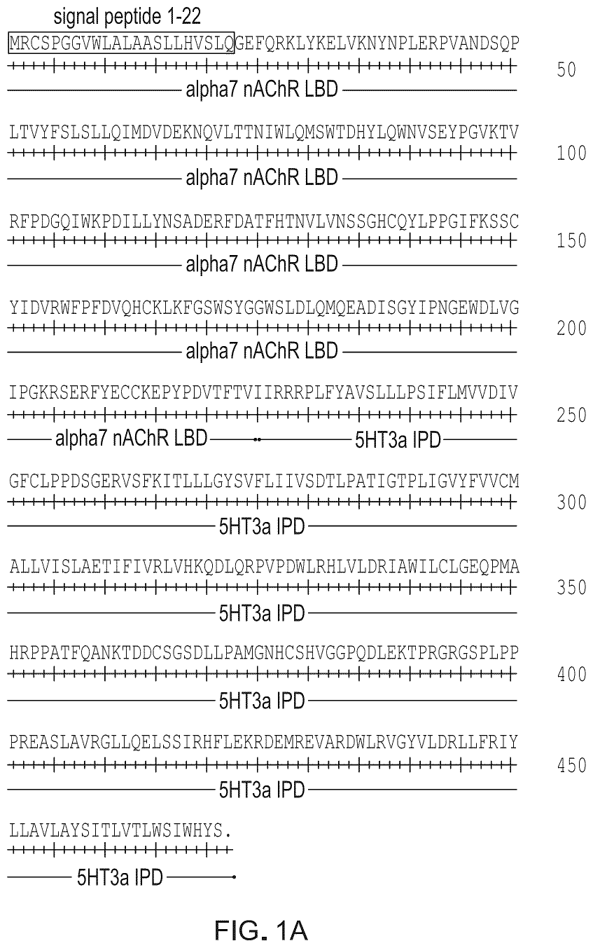

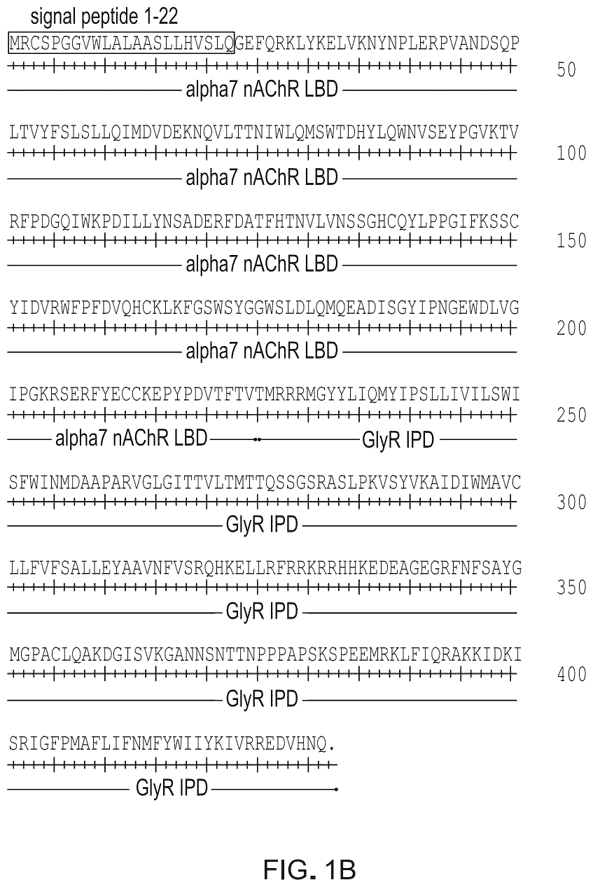

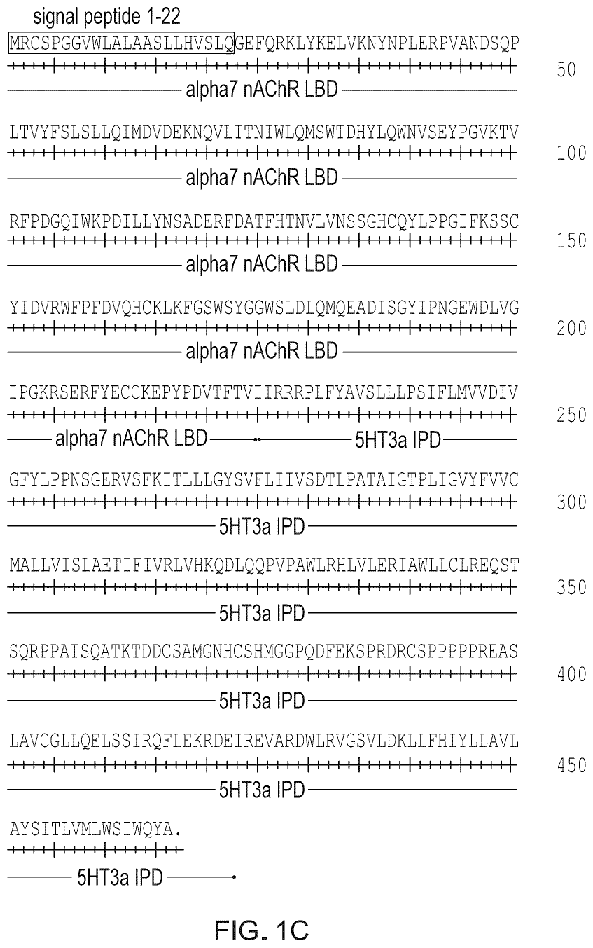

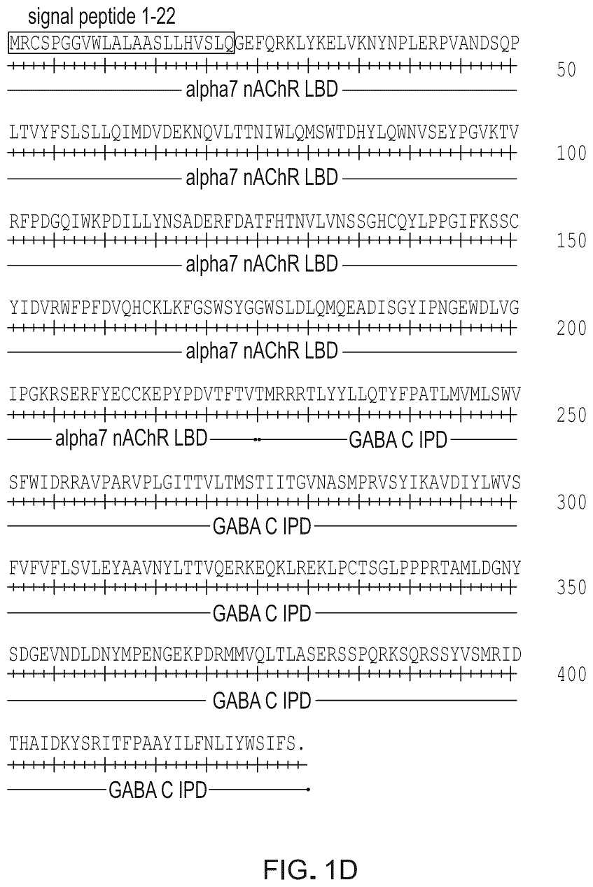

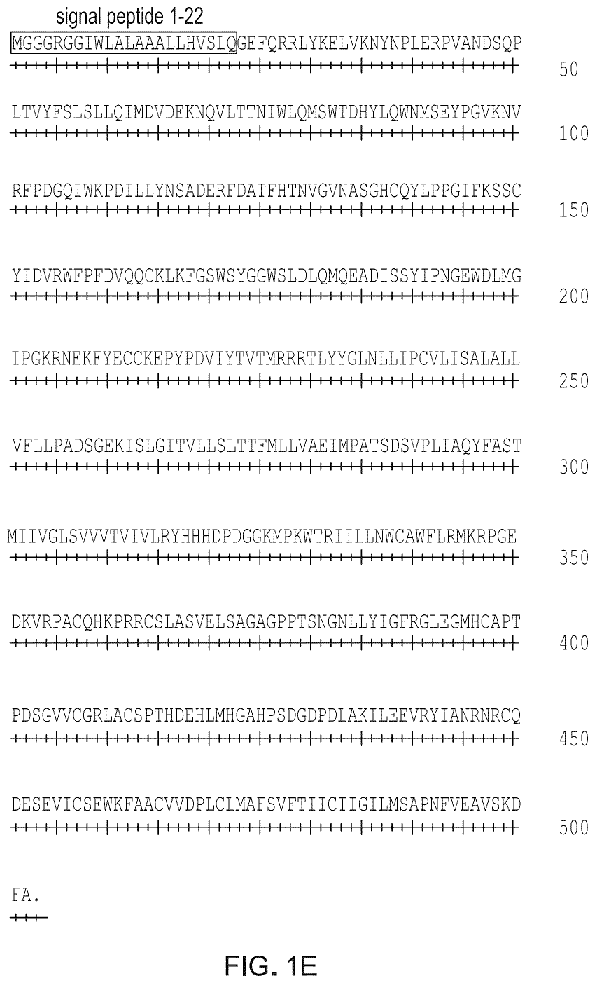

In cases where a modified LGIC subunit described herein is a chimeric LGIC subunit, the chimeric LGIC subunit can include varied fusion points connecting the LBD and the IPD such that the number of amino acids in a LBD may vary when the LBD is fused with different IPDs to form a chimeric channel subunit. For example, the length of an α7 nAChR LBD used to form a chimeric LGIC subunit with a 5HTS IPD is different from the length of an α7 nAChR LBD used to form a chimeric LGIC subunit with a GlyR IPD (compare, for example, FIGS. 1A and 1C to FIG. 1B).

A modified LGIC subunit described herein can include a LBD having at least one modified amino acid and/or an IPD having at least one modified amino acid. For example, a modified LGIC subunit described herein can include a α7 LBD having at least 75 percent sequence identity to SEQ ID NO:1, SEQ ID NO:2, SEQ ID NO: 11, or SEQ ID NO: 12, and an amino acid substitution at amino acid residue 27, 41, 77, 79, 131, 139, 141, 175, 210, 216, 217, and/or 219. For example, a modified LGIC subunit described herein can include a GlyR IPD having at least 75 percent sequence identity to a sequence set forth in SEQ ID NO:5, and an amino acid substitution at amino acid residue 298 of an α7-GlyR chimeric receptor (e.g., SEQ ID NO:7). For example, a modified LGIC subunit described herein can include a GABAc IPD having at least 75 percent sequence identity to SEQ ID NO:9, and an amino acid substitution at amino acid residue 298 of an α7-GABAc chimeric receptor (e.g., SEQ ID NO: 10). In some cases, a modified LGIC subunit described herein can include more than one (e.g., two, three, four, five, six, seven, eight, nine, ten, eleven, twelve, or more) amino acid modifications. The modification can be an amino acid substitution. In some cases, the modified amino acid can confer pharmacological selectivity to the modified LGIC. For example, the modified amino acid can confer the modified LGIC with selective binding of an exogenous LGIC ligand. For example, the modified amino acid can confer the modified LGIC with reduced (minimized or eliminated) binding of an unmodified LGIC subunit (an LGIC subunit lacking the modification and/or an endogenous LGIC subunit). For example, the modified amino acid can confer the modified LGIC with reduced (minimized or eliminated) binding of an endogenous LGIC ligand.

In some aspects, a modified LGIC subunit described herein can include at least one modified amino acid that confers the modified LGIC with selective binding (e.g., enhanced binding or increased potency) with an exogenous LGIC ligand. The binding with an exogenous LGIC ligand can be selective over the binding with an endogenous LGIC ligand. A modified LGIC subunit with selective binding with an exogenous LGIC ligand can include any appropriate LDB (e.g., a α7 LBD). In some aspects, the modified LGIC subunit can include a α7 LBD set forth in SEQ ID NO: 1, SEQ ID NO:2, SEQ ID NO: 11, or SEQ ID NO: 12, and the amino acid modification can be a substitution at amino acid residue 77, 79, 131 139, 141, 175, and/or 216. In some cases, the tryptophan at amino acid residue 77 of SEQ ID NO:1, SEQ ID NO:2, SEQ ID NO: 11, or SEQ ID NO: 12 can be substituted with a hydrophobic amino acid residue such as phenylalanine (e.g., W77F), tyrosine (e.g., W77Y), or methionine (e.g., W77M). For example, a modified LGIC subunit described herein can include a α7 LBD set forth in SEQ ID NO:1, SEQ ID NO:2, SEQ ID NO: 11, or SEQ ID NO: 12 and having a W77F substitution. In some cases, the glutamine at amino acid residue 79 of SEQ ID NO:1, SEQ ID NO:2, SEQ ID NO: 11, or SEQ ID NO: 12 can be substituted with an amino acid residue such as alanine (e.g., Q79A), glycine (e.g., Q79G), or serine (e.g., Q79S). For example, a modified LGIC subunit described herein can include a α7 LBD having a Q79G substitution. In some cases, the leucine at amino acid residue 131 of SEQ ID NO: 1, SEQ ID NO:2, SEQ ID NO:11, or SEQ ID NO: 12 can be substituted with an amino acid residue such as alanine (e.g., L131A), glycine (e.g., L131G), methionine (e.g., L131M), asparagine (e.g., L13IN), glutamine (e.g., L131Q), valine (e.g., L131V), or phenylalanine (e.g., L131F). In some cases, the glycine at amino acid residue 175 of SEQ ID NO:1, SEQ ID NO: 2, SEQ ID NO: 11, or SEQ ID NO: 12 can be substituted with an amino acid residue such as lysine (e.g., G175K), alanine (e.g., G175A), phenylalanine (e.g., G175F), histidine (e.g., G175H), methionine (e.g., G175m), arginine (e.g., G175R), serine (e.g., G175S), valine (e.g., G175V). In some cases, the proline at amino acid residue 216 of SEQ ID NO:1, SEQ ID NO: 2, SEQ ID NO:11, or SEQ ID NO: 12 can be substituted with an amino acid residue such as isoleucine (e.g., P216I). A modified LGIC subunit with selective binding with an exogenous LGIC ligand can include any appropriate IPD (e.g., a GlyR IPD or a GABAA-P IPD). In some aspects, the modified LGIC subunit can include a GlyR IPD set forth in SEQ ID NO: 5, and the amino acid modification can be a substitution at amino acid residue 298 of an α7-GlyR chimeric receptor (e.g., SEQ ID NO:7). In some cases, the alanine at amino acid residue 298 of SEQ ID NO:7 can be substituted with an amino acid residue such as glycine (e.g., A298G). In some aspects, the modified LGIC subunit can include the a GABAA-P IPD set forth in SEQ ID NO:9, and the amino acid modification can be a substitution at amino acid residue 298 of an α7-GABAA-p chimeric receptor (e.g., SEQ ID NO: 10). In some cases, the tryptophan at amino acid residue 298 of SEQ ID NO: 10 can be substituted with an amino acid residue such as alanine (e.g., W298A).

In some cases, a modified LGIC subunit described herein can include more than one (e.g., two, three, four, five, six, seven, eight, nine, ten, eleven, twelve, or more) amino acid modifications. For example, a modified LGIC subunit described herein can have at least 75 percent sequence identity to SEQ ID NO:7 and can include a Q79G substitution and a A298G substitution. Additional examples of modifications that can confer the modified LGIC with selective binding of an exogenous LGIC ligand include modifications described elsewhere (see, e.g., U.S. Pat. No. 8,435,762).

A modified LGIC subunit that selectively binds (e.g., enhanced binding or increased potency) an exogenous LGIC ligand over an endogenous (e.g., a canonical) LGIC ligand can also be described as having enhanced potency for an exogenous ligand. In some cases, a modified LGIC subunit described herein that selectively binds an exogenous LGIC ligand can have at least 4 fold (e.g., at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, or at least 20 fold) enhanced potency for an exogenous ligand. In some cases, a modified LGIC subunit described herein that selectively binds an exogenous LGIC ligand can have about 4 fold to about 200 fold (e.g., about 4 fold to about 200 fold, about 5 fold to about 180 fold, about 6 fold to about 175 fold, about 7 fold to about 150 fold, about 8 fold to about 125 fold, about 9 fold to about 100 fold, about 10 fold to about 90 fold, about 11 fold to about 75 fold, about 12 fold to about 65 fold, about 13 fold to about 50 fold, about 14 fold to about 40 fold, or about 15 fold to about 30 fold) enhanced potency for an exogenous ligand. For example, a modified LGIC subunit described herein that selectively binds an exogenous LGIC ligand can have about 10 fold to about 100 fold enhanced potency for an exogenous ligand. For example, a modified LGIC subunit described herein that selectively binds an exogenous LGIC ligand can have about 10 fold to about 20 fold enhanced potency for an exogenous ligand.

In some aspects, a modified LGIC subunit described herein can include at least one modified amino acid that confers the modified LGIC with reduced (e.g., minimized or eliminated) binding with an unmodified LGIC subunit. The binding with a modified LGIC subunit having the same modification can be selective over the binding with an unmodified LGIC subunit. An unmodified LGIC subunit can be a LGIC subunit lacking the modification that confers the modified LGIC with reduced binding with an unmodified LGIC subunit or an unmodified LGIC can be an endogenous LGIC subunit. The modification that confers the modified LGIC with reduced binding with an unmodified LGIC subunit can be a charge reversal modification. A modified LGIC subunit with reduced binding with an unmodified LGIC subunit can include any appropriate LBD (e.g., a α7 LBD). In some aspects, the modified LGIC subunit can include a α7 LBD set forth in SEQ ID NO:1, SEQ ID NO:2, SEQ ID NO: 11, or SEQ ID NO: 12, and the amino acid modification can be a substitution at amino acid residue 27 and/or 41. For example, the arginine at amino acid residue 27 of SEQ ID NO: 1, SEQ ID NO:2, SEQ ID NO: 11, or SEQ ID NO: 12 can be substituted with an aspartic acid (e.g., R27D). For example, the glutamic acid at amino acid residue 41 of SEQ ID NO: 1, SEQ ID NO:2, SEQ ID NO:11, or SEQ ID NO: 12 can be substituted with an arginine (e.g., E41R). In some cases, a modified LGIC subunit described herein can include a α7 LBD having a R27D substitution and a E41R.

In some aspects, a modified LGIC subunit described herein can include at least one modified amino acid that confers the modified LGIC with reduced (e.g., minimized or eliminated) binding of an endogenous LGIC ligand. The endogenous LGIC ligand can be ACh. A modified LGIC subunit with reduced binding of an endogenous LGIC ligand can include any appropriate IPD (e.g., a GlyR LBD). For example, the modified LGIC subunit can include a α7 LBD set forth in SEQ ID NO:1, SEQ ID NO:2, SEQ ID NO: 11, or SEQ ID NO: 12, and the amino acid modification can be a substitution at amino acid residue 115, 131, 139, 210, 217 and/or 219. In some cases, the tyrosine at amino acid residue 115 of SEQ ID NO: 1, SEQ ID NO:2, SEQ ID NO:11, or SEQ ID NO: 12 can be substituted with a phenylalanine (e.g., Y115F). In some cases, the leucine at amino acid residue 131 of SEQ ID NO: 1, SEQ ID NO:2, SEQ ID NO:11, or SEQ ID NO: 12 can be substituted with an amino acid residue such as alanine (e.g., L131A), glycine (e.g., L131G), methionine (e.g., L131M), asparagine (e.g., L13IN), glutamine (e.g., L131Q), valine (e.g., L131V), or phenylalanine (e.g., L131F). In some cases, the glutamine at amino acid residue 139 of SEQ ID NO:1, SEQ ID NO: 2, SEQ ID NO:11, or SEQ ID NO: 12 can be substituted with a glycine (e.g., Q139G) or a leucine (e.g., Q139L). In some cases, the tyrosine at amino acid residue 210 of SEQ ID NO: 1, SEQ ID NO:2, SEQ ID NO:11, or SEQ ID NO: 12 can be substituted with a phenylalanine (e.g., Y210F). In some cases, the tyrosine at amino acid residue 217 of SEQ ID NO: 1, SEQ ID NO:2, SEQ ID NO: 11, or SEQ ID NO: 12 can be substituted with a phenylalanine (e.g., Y217F). In some cases, the aspartate at amino acid residue 219 of SEQ ID NO: 1, SEQ ID NO:2, SEQ ID NO: 11, or SEQ ID NO: 12 can be substituted with an alanine (e.g., D219A).

In some aspects, a modified LGIC subunit described herein can include at least one modified amino acid that confers the modified LGIC with increased ion conductance. In some cases, the modified LGIC subunit can include a 5HT3 IPD set forth in SEQ ID NO:3, and the amino acid modification can be a substitution at amino acid residue 425, 429, and/or 433. For example, a modified LGIC subunit described herein can include a 5HT3 IPD having a R425Q substitution, a R429D substitution, and a R433A substitution. In some cases, the modified LGIC subunit can include a 5HT3 IPD set forth in SEQ ID NO:4, and the amino acid modification can be a substitution at amino acid residue 420, 424, and/or 428. For example, a modified LGIC subunit described herein can include a 5HT3 IPD having a R420Q substitution, a R424D substitution, and a R428A substitution.

In some cases, a modified LGIC described herein can include at least one chimeric α7-5HT3 LGIC subunit (SEQ ID NO:6) having a human α7 nAChR LBD (SEQ ID NO:1) with a Q79G amino acid substitution and a Y115F amino acid substitution, and a murine 5HT3 IPD (SEQ ID NO:3).

In some cases, a modified LGIC described herein can include at least one chimeric α7-5HT3 LGIC subunit (SEQ ID NO:6) having a human α7 nAChR LBD (SEQ ID NO:1) with a Q79G amino acid substitution and a Q139G amino acid substitution, and a murine 5HT3 IPD (SEQ ID NO:3).

In some cases, a modified LGIC described herein can include at least one chimeric α7-GlyR LGIC subunit (SEQ ID NO:7) having a human α7 nAChR LBD (SEQ ID NO:2) with a Q79G amino acid substitution and a Y115F amino acid substitution, and a human GlyR IPD (SEQ ID NO:5) with a A298G amino acid substitution.

In some cases, a modified LGIC described herein can include at least one chimeric α7-GlyR LGIC subunit (SEQ ID NO:7) having a human α7 nAChR LBD (SEQ ID NO:2) with a Q79G amino acid substitution and a Q139G amino acid substitution, and a human GlyR IPD (SEQ ID NO:5) with a A298G amino acid substitution.

In some cases, a modified LGIC described herein can include at least one chimeric α7-GlyR LGIC subunit (SEQ ID NO:7) having a human α7 nAChR LBD (SEQ ID NO:2) with a R27D amino acid substitution, a E41R amino acid substitution, a Q79G amino acid substitution, and a Y115F amino acid substitution, and a human GlyR IPD (SEQ ID NO:5) with a A298G amino acid substitution.

In some cases, a modified LGIC described herein can include at least one chimeric α7-GlyR LGIC subunit (SEQ ID NO:7) having a human α7 nAChR LBD (SEQ ID NO:2) with a substitution at amino acid residue 131 (e.g., L131G, L131A, L131M, or L13IN), and a human GlyR IPD (SEQ ID NO:5).

In some cases, a modified LGIC described herein can include at least one chimeric α7-GlyR LGIC subunit (SEQ ID NO:7) having a human α7 nAChR LBD (SEQ ID NO:2) with a substitution at amino acid residues 131 (e.g., L131G, L131A, L131M, or L131N) and Y115 (e.g., Y115F), and a human GlyR IPD (SEQ ID NO:5).

In some cases, a modified LGIC described herein can include at least one chimeric α7-GlyR LGIC subunit (SEQ ID NO:7) having a human α7 nAChR LBD (SEQ ID NO:2) with a substitution at amino acid residues 131 (e.g., L131G, L131A, L131M, or L131N) and 139 (e.g., Q139L), and a human GlyR IPD (SEQ ID NO:5).

In some cases, a modified LGIC described herein can include at least one chimeric α7-GlyR LGIC subunit (SEQ ID NO:7) having a human α7 nAChR LBD (SEQ ID NO:2) with a substitution at amino acid residues 131 (e.g., L131G, L131A, L131M, or L131N) and 217 (e.g., Y217F), and a human GlyR IPD (SEQ ID NO:5).

In some cases, a modified LGIC described herein can include at least one chimeric α7-GlyR LGIC subunit (SEQ ID NO:7) having a human α7 nAChR LBD (SEQ ID NO:2) with a substitution at amino acid residues 131 (e.g., L131G, L131A, L131M, or L131N), 139 (e.g., Q139L), and 217 (e.g., Y217F), and a human GlyR IPD (SEQ ID NO:5).

In some cases, a modified LGIC described herein can include at least one chimeric α7-5HT3 LGIC subunit having a human α7 nAChR LBD (SEQ ID NO:2) with a substitution at amino acid residue 131 (e.g., L131G, L131A, L131M, or L131N), and a human 5HT3 IPD (SEQ ID NO:4).

In some cases, a modified LGIC described herein can include at least one chimeric α7-GlyR LGIC subunit (SEQ ID NO:7) having a human α7 nAChR LBD (SEQ ID NO:2) with a substitution at amino acid residue 175 (e.g., G175K), and a human GlyR IPD (SEQ ID NO: 5).

In some cases, a modified LGIC described herein can include at least one chimeric α7-5HT3 LGIC subunit having a human α7 nAChR LBD (SEQ ID NO:2) with a substitution at amino acid residue 131 (e.g., L131G, L131A, L131M, or L131N) and 139 (e.g., Q139L), and a human 5HT3 IPD (SEQ ID NO:4) with a R420Q substitution, a R424D substitution, and a R428A substitution.

In some cases, a modified LGIC described herein can include at least one chimeric α7-5HT3 LGIC subunit having a human α7 nAChR LBD (SEQ ID NO:2) with a substitution at amino acid residue 131 (e.g., L131G, L131A, L131M, or L131N) and 139 (e.g., Q139L) and 217 (e.g., Y217F), and a human 5HT3 IPD (SEQ ID NO:4) with a R420Q substitution, a R424D substitution, and a R428A substitution.

In some cases, a modified LGIC described herein can include at least one chimeric α7-GlyR LGIC subunit (SEQ ID NO:7) having a human α7 nAChR LBD (SEQ ID NO:2) with a substitution at amino acid residues 175 (e.g., G175K) and 115 (e.g., Y115F), and a human GlyR IPD (SEQ ID NO:5).

In some cases, a modified LGIC described herein can include at least one chimeric α7-GlyR LGIC subunit (SEQ ID NO:7) having a human α7 nAChR LBD (SEQ ID NO:2) with a substitution at amino acid residues 175 (e.g., G175K) and 115 (e.g., Y115F) and 79 (e.g., Q79G), and a human GlyR IPD (SEQ ID NO:5).

In some cases, a modified LGIC described herein can include at least one chimeric α7-GlyR LGIC subunit (SEQ ID NO:7) having a human α7 nAChR LBD (SEQ ID NO:2) with a substitution at amino acid residues 175 (e.g., G175K) and 77 (e.g., W77F) and 79 (e.g., Q79G), and a human GlyR IPD (SEQ ID NO:5).

In some cases, a modified LGIC described herein can include at least one chimeric α7-GlyR LGIC subunit (SEQ ID NO:7) having a human α7 nAChR LBD (SEQ ID NO:2) with a substitution at amino acid residue 216 (e.g., P216I), and a human GlyR IPD (SEQ ID NO: 5).

In some cases, a modified LGIC described herein can include at least one chimeric α7-GlyR LGIC subunit (SEQ ID NO:7) having a human α7 nAChR LBD (SEQ ID NO:2) with a substitution at amino acid residues 216 (e.g., P216I) and 79 (e.g., Q79G), and a human GlyR IPD (SEQ ID NO:5).

In some cases, a modified LGIC described herein can include at least one chimeric α7-GlyR LGIC subunit (SEQ ID NO: 10) having a human α7 nAChR LBD (SEQ ID NO:2) with a substitution at amino acid residue 131 (e.g., L131A, L131G, L131M, L13IN, L131Q, L131V, or L131F), and a human GABAc IPD (SEQ ID NO:9).

In some cases, a modified LGIC described herein can include one or more additional polypeptide sequences. A polypeptide sequence can be a transport sequence (e.g., an export sequence and/or a signal sequence). Examples of export sequences include, without limitation, ER export sequences (e.g., FCYENEV (SEQ ID NO:16)). Examples of signal sequences include, without limitation, CHRNB4 signal sequences (e.g., MRRAPSLVLFFLVALCGRGNC (SEQ ID NO:17)). A polypeptide sequence can be a targeting sequence. Examples of targeting sequences include, without limitation, KCNB1 somatic targeting sequences (e.g., QSQPILNTKEMAPQSKPPEELEMSSMPSPVAPLPARTEGVIDMRSMSSIDSFISCATDFP EATRF (SEQ ID NO:18)). The one or more additional polypeptide sequences can be included in a modified LGIC in any appropriate location. In some cases, an additional polypeptide sequence can be a terminal (e.g., a C-terminal or an N-terminal) polypeptide sequence. In some cases, an additional polypeptide sequence can be an insertion. In some cases, an additional polypeptide sequence can be a substitution.

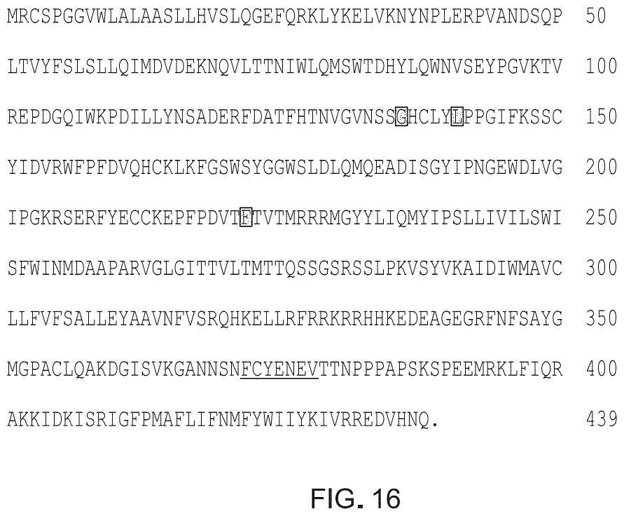

In some cases, a modified LGIC described herein can include an export sequence. For example, a modified LGIC can include an ER export sequence (e.g., FCYENEV (SEQ ID NO: 16). An exemplary modified LGIC containing a α7 nAChR LBD having a L131G substitution, a Q139L substitution, and a Y217F (e.g., each of which is relative to the residue numbering set forth in SEQ ID NO: 1, SEQ ID NO:2, SEQ ID NO: 11, or SEQ ID NO:12), and a GlyR IPD having an ER export sequence inserted between residues 142 and 143 (e.g., relative to the residue numbering set forth in SEQ ID NO:5) is shown in FIG. 16.

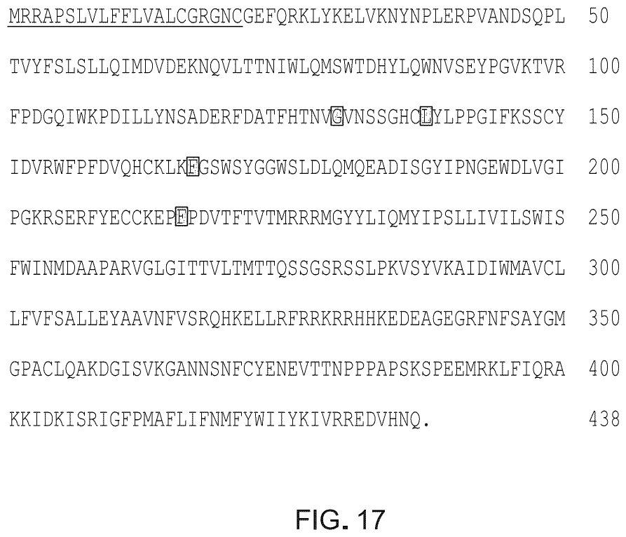

In some cases, a modified LGIC described herein can include a signal sequence. for example, a modified LGIC can include a CHRNB4 signal sequence (e.g., MRRAPSLVLFFLVALCGRGNC (SEQ ID NO:17). An exemplary modified LGIC containing a α7 nAChR LBD having a CHRNB4 signal sequence substituting residues 1-22, a L131G substitution, a Q139L substitution, and a Y217F (e.g., each of which is relative to the residue numbering set forth in SEQ ID NO:1, SEQ ID NO:2, SEQ ID NO: 11, or SEQ ID NO: 12), and a GlyR IPD is shown in FIG. 17.

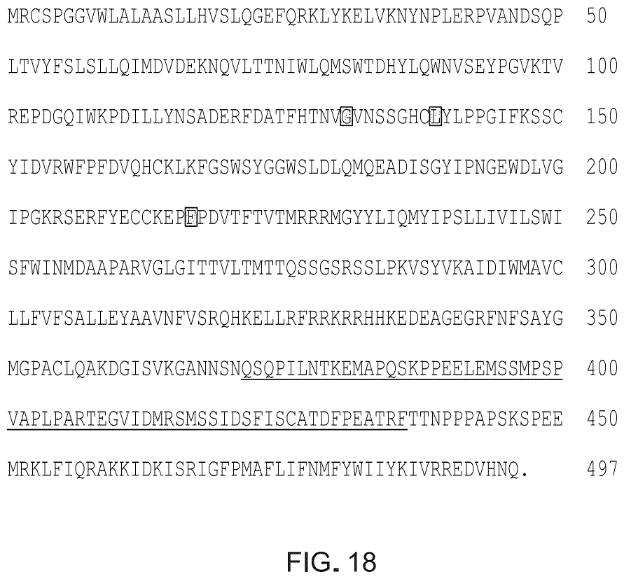

In some cases, a modified LGIC described herein can include a targeting sequence. For example, a modified LGIC can include a KCNB1 somatic targeting sequence (e.g., QSQPILNTKEMAPQSKPPEELEMSSMPSPVAPLPARTEGVIDMRSMSSIDSFISCATDFP EATRF (SEQ ID NO: 18). An exemplary modified LGIC containing a α7 nAChR LBD having a L131G substitution, a Q139L substitution, and a Y217F (e.g., each of which is relative to the residue numbering set forth in SEQ ID NO:1, SEQ ID NO:2, SEQ ID NO:11, or SEQ ID NO:12), and a GlyR IPD having a KCNB1 somatic targeting sequence inserted between residues 142 and 143 (e.g., relative to the residue numbering set forth in SEQ ID NO: 5) is shown in FIG. 18. In cases where a LBD and/or a IPD is a homolog, orthologue, or paralog of a sequence set forth herein (e.g., SEQ ID NOs: 1-5 and/or 9), it is understood that reference to a particular modified amino acid residue can shift to the corresponding amino acid in the homolog, orthologue, or paralog. For example, residues 425, 429, and 433 in a murine 5HT3 IPD set forth in SEQ ID NO:3 correspond to residues 420, 424, and 428 in a human 5HT3 IPD set forth in SEQ ID NO:4, and the R425Q, R429D, and R433A substitutions in a murine 5HT3 IPD correspond to R420Q, R424D, and R428A substitutions in a human 5HT3 IPD.

Any method can be used to obtain a modified LGIC subunit described herein. In some cases, peptide synthesis methods can be used to make a modified LGIC subunit described herein. Examples of methods of peptide synthesis include, without limitation, liquid-phase peptide synthesis, and solid-phase peptide synthesis. In some cases, protein biosynthesis methods can be used to make a modified LGIC subunit described herein. Examples of methods of protein biosynthesis include, without limitation, transcription and/or translation of nucleic acids encoding a phosphorylation-mimicking peptide provided herein. Similar modified LGIC subunits (e.g., modified subunits having essentially the same modifications and/or having essentially the same amino acid sequence) will self-assemble through interactions between the LBDs to form a modified LGIC.

This document also provides nucleic acids encoding modified LGIC subunits described herein as well as constructs (e.g., synthetic constructs such as plasmids, non-viral vectors, viral vectors (such as adeno-associated virus, a herpes simplex virus, or lentivirus vectors)) for expressing nucleic acids encoding modified LGIC subunits described herein.

A nucleic acid sequence encoding modified LGIC subunit described herein can encode any LGIC described herein. In some cases, a nucleic acid sequence provided herein can encode a LBD from any LGIC described herein. In some cases, a nucleic acid sequence provided herein can encode an IPD from any LGIC described herein. In cases where a nucleic acid sequence provided herein encodes a chimeric LGIC, the chimeric LGIC can include a LBD selected from any appropriate LGIC and an IPD selected from any appropriate LGIC.

In some cases, a nucleic acid sequence can encode a LGIC described herein. For example, a nucleic acid sequence can encode a nAChR (e.g., a α7 nAChR). A representative nucleic acid sequence encoding a rat α7 nAChR amino acid sequence (including both a LBD and an IPD) is as follows.

SEQ ID NO: 19
ATGGGCGGCGGGCGGGGAGGCATCTGGCTGGCTCTGGCCGCGGCGCTGCT
GCACGTGTCCCTGCAAGGCGAGTTCCAGAGGAGGCTGTACAAGGAGCTGG
TCAAGAACTACAACCCGCTGGAGAGGCCGGTGGCCAACGACTCGCAGCCG
CTCACCGTGTACTTCTCCCTGAGTCTCCTGCAGATCATGGATGTGGATGA
GAAGAACCAAGTTTTAACCACCAACATTTGGCTACAAATGTCTTGGACAG
ATCACTATTTGCAGTGGAACATGTCTGAGTACCCCGGAGTGAAGAATGTT
CGTTTTCCAGATGGCCAGATTTGGAAACCAGACATTCTCCTCTATAACAG
TGCTGATGAGCGCTTTGATGCCACGTTCCACACCAATGTTTTGGTGAATG
CATCTGGGCATTGCCAGTATCTCCCTCCAGGCATATTCAAGAGCTCCTGC
TACATTGACGTTCGCTGGTTCCCTTTTGATGTGCAGCAGTGCAAACTGAA
GTTTGGGTCCTGGTCCTATGGAGGGTGGTCACTGGACCTGCAAATGCAAG
AGGCAGATATCAGCAGCTATATCCCCAACGGAGAATGGGATCTCATGGGA
ATCCCTGGCAAAAGGAATGAGAAGTTCTATGAGTGCTGCAAAGAGCCATA
CCCAGATGTCACCTACACAGTAACCATGCGCCGTAGGACACTCTACTATG
GCCTCAATCTGCTCATCCCTTGTGTACTCATTTCAGCCCTGGCTCTGCTG
GTATTCTTGCTGCCTGCAGACTCTGGAGAGAAAATCTCTCTTGGAATAAC
TGTCTTACTTTCTCTGACTGTCTTCATGCTGCTTGTGGCTGAGATCATGC
CAGCAACATCTGATTCTGTGCCCTTGATAGCACAATACTTCGCCAGCACC
ATGATCATCGTGGGCCTCTCTGTAGTGGTGACAGTGATTGTGCTGAGATA
TCACCACCATGACCCTGATGGTGGCAAAATGCCTAAGTGGACCAGAATCA
TTCTCCTGAACTGGTGTGCATGGTTTCTGCGCATGAAGAGGCCCGGAGAG
GACAAGGTGCGGCCAGCTTGTCAGCACAAGCCTCGGCGCTGCAGCCTGGC
CAGTGTGGAGCTGAGTGCAGGTGCTGGGCCACCCACCAGCAATGGCAACC
TGCTCTACATTGGCTTCCGAGGCCTGGAGGGCATGCACTGTGCCCCAACT
CCAGACTCTGGGGTCGTATGTGGCCGTTTGGCCTGCTCCCCAACACATGA
TGAGCACCTCATGCACGGTGCACACCCCTCTGATGGGGACCCCGACCTGG
CCAAGATCCTGGAGGAGGTCCGCTACATCGCCAACCGCAACCGCTGCCAG
GACGAGAGTGAGGTGATCTGCAGTGAATGGAAGTTTGCAGCCTGCGTGGT
GGACCCGCTTTGCCTCATGGCCTTTTCGGTCTTTACCATCATCTGTACCA
TCGGCATCCTCATGTCAGCTCCAAACTTTGTGGAGGCTGTGTCCAAAGAC
TTTGCTTAA

In some cases, a nucleic acid sequence encoding a modified LGIC subunit described herein can encode a LBD from a α7 nAChR. Examples of nucleic acid sequences encoding α7 nAChR LBDs include, without limitation, a nucleic acid sequence set forth in SEQ ID NO: 20, a nucleic acid sequence set forth in SEQ ID NO:21, and a nucleic acid sequence set forth in SEQ ID NO:22. In some cases, a nucleic acid sequence encoding α7 nAChR LBD can have at least 75 percent sequence identity (e.g., at least 80%, at least 82%, at least 85%, at least 88%, at least 90%, at least 93%, at least 95%, at least 97% or at least 99% sequence identity) to SEQ ID NO:20, SEQ ID NO:21, or SEQ ID NO:22.

SEQ ID NO: 20
ATGCGCTGTTCTCCAGGCGGCGTGTGGCTCGCCCTGGCTGCTTCCCTTCT
GCACGTTAGCCTGCAGGGTGAGTTCCAGCGCAAACTGTATAAGGAGCTTG
TTAAGAATTATAACCCCCTGGAGCGGCCGGTCGCAAATGATTCCCAGCCA
CTGACAGTGTACTTCAGCCTCTCCTTGCTGCAGATCATGGACGTGGATGA
AAAGAACCAGGTGCTGACCACTAATATTTGGTTGCAGATGTCCTGGACCG
ATCACTACTTGCAGTGGAATGTGAGCGAATACCCAGGTGTAAAGACTGTA
AGATTCCCTGACGGCCAAATCTGGAAACCAGATATCCTGCTGTACAACAG
CGCAGACGAAAGGTTTGATGCAACATTTCACACCAACGTGTTGGTCAATT
CTTCAGGCCACTGCCAGTACCTGCCCCCTGGAATCTTCAAGTCCTCATGC
TATATCGACGTCCGCTGGTTTCCCTTCGACGTCCAGCACTGCAAACTCAA
ATTCGGGAGCTGGAGCTACGGCGGATGGAGCCTGGATCTGCAAATGCAGG
AGGCTGACATCTCTGGTTACATCCCGAATGGGGAGTGGGACCTTGTGGGA
ATCCCCGGTAAAAGAAGCGAGCGATTTTATGAATGCTGCAAGGAACCCTA
CCCTGACGTAACATTCACAGTT
SEQ ID NO: 21
ATGCGCTGTTCTCCAGGCGGCGTGTGGCTCGCCCTGGCTGCTTCCCTTCT
GCACGTTAGCCTGCAGGGTGAGTTCCAGCGCAAACTGTATAAGGAGCTTG
TTAAGAATTATAACCCCCTGGAGCGGCCGGTCGCAAATGATTCCCAGCCA
CTGACAGTGTACTTCAGCCTCTCCTTGCTGCAGATCATGGACGTGGATGA
AAAGAACCAGGTGCTGACCACTAATATTTGGTTGCAGATGTCCTGGACCG
ATCACTACTTGCAGTGGAATGTGAGCGAATACCCAGGTGTAAAGACTGTA
AGATTCCCTGACGGCCAAATCTGGAAACCAGATATCCTGCTGTACAACAG
CGCAGACGAAAGGTTTGATGCAACATTTCACACCAACGTGTTGGTCAATT
CTTCAGGCCACTGCCAGTACCTGCCCCCTGGAATCTTCAAGTCCTCATGC
TATATCGACGTCCGCTGGTTTCCCTTCGACGTCCAGCACTGCAAACTCAA
ATTCGGGAGCTGGAGCTACGGCGGATGGAGCCTGGATCTGCAAATGCAGG
AGGCTGACATCTCTGGTTACATCCCGAATGGGGAGTGGGACCTTGTGGGA
ATCCCCGGTAAAAGAAGCGAGCGATTTTATGAATGCTGCAAAGAGCCCTA
CCCAGATGTCACCTTCACAGTGACCATGCGGAGACGC
SEQ ID NO: 22
ATGCGCTGTTCTCCAGGCGGCGTGTGGCTCGCCCTGGCTGCTTCCCTTCT
GCACGTTAGCCTGCAGGGTGAGTTCCAGCGCAAACTGTATAAGGAGCTTG
TTAAGAATTATAACCCCCTGGAGCGGCCGGTCGCAAATGATTCCCAGCCA
CTGACAGTGTACTTCAGCCTCTCCTTGCTGCAGATCATGGACGTGGATGA
AAAGAACCAGGTGCTGACCACTAATATTTGGTTGCAGATGTCCTGGACCG
ATCACTACTTGCAGTGGAATGTGAGCGAATACCCAGGTGTAAAGACTGTA
AGATTCCCTGACGGCCAAATCTGGAAACCAGATATCCTGCTGTACAACAG
CGCAGACGAAAGGTTTGATGCAACATTTCACACCAACGTGTTGGTCAATT
CTTCAGGCCACTGCCAGTACCTGCCCCCTGGAATCTTCAAGTCCTCATGC
TATATCGACGTCCGCTGGTTTCCCTTCGACGTCCAGCACTGCAAACTCAA
ATTCGGGAGCTGGAGCTACGGCGGATGGAGCCTGGATCTGCAAATGCAGG
AGGCTGACATCTCTGGTTACATCCCGAATGGGGAGTGGGACCTTGTGGGA
ATCCCCGGTAAAAGAAGCGAGCGATTTTATGAATGCTGCAAAGAGCCCTA
CCCAGATGTCACCTTCACAGTGACCATGCGGAGACGCACACTGTATTAC

In some cases, a nucleic acid sequence encoding a modified LGIC subunit described herein can encode an IPD from a 5HT3 receptor. Examples of nucleic acid sequences encoding 5HT3 IPDs include, without limitation, a nucleic acid sequence set forth in SEQ ID NO: 23, and a nucleic acid sequence set forth in SEQ ID NO:24. In some cases, a nucleic acid sequence encoding a 5HT3 IPD can have at least 75 percent sequence identity (e.g., at least 80%, at least 82%, at least 85%, at least 88%, at least 90%, at least 93%, at least 95%, at least 97% or at least 99% sequence identity) to SEQ ID NO:23 or SEQ ID NO:24.

SEQ ID NO: 23
ATCATCAGAAGAAGGCCATTGTTCTACGCCGTTAGTTTGTTGCTCCCCAG
TATTTTTCTCATGGTCGTGGACATCGTGGGATTTTGTCTCCCACCTGATA
GCGGGGAGAGGGTCTCCTTTAAGATTACCTTGTTGCTCGGCTATTCTGTA
TTTCTGATCATCGTGTCCGATACCCTTCCTGCCACAATCGGCACTCCGCT
GATAGGAGTGTATTTCGTCGTGTGTATGGCACTCCTGGTGATAAGTCTGG
CGGAAACTATCTTCATTGTACGGCTGGTACATAAGCAGGACCTGCAAAGA
CCCGTGCCAGACTGGTTGCGACACCTTGTGCTGGACAGAATTGCATGGAT
TCTGTGTCTTGGCGAGCAACCTATGGCCCACCGGCCACCTGCAACCTTTC
AAGCCAACAAGACAGACGATTGTAGTGGGTCTGATCTGTTGCCTGCTATG
GGGAATCACTGCTCCCATGTTGGGGGACCACAAGATTTGGAAAAGACCCC
ACGGGGGCGGGGATCACCCCTTCCTCCTCCCCGAGAAGCCTCTCTCGCTG
TCCGGGGGCTGCTCCAGGAACTGTCAAGCATCCGACATTTTCTGGAGAAG
CGGGACGAGATGAGGGAAGTCGCTAGAGACTGGCTGCGAGTGGGCTACGT
CCTTGACAGGCTGCTGTTTCGGATCTACTTGCTGGCGGTGCTGGCTTATT
CCATTACTCTGGTGACACTCTGGTCCATATGGCACTACAGTTAG
SEQ ID NO: 24
ATCATCCGTAGAAGGCCTCTGTTTTACGTGGTGAGCCTGCTGCTGCCATC
CATCTTCCTGATGGTCATGGACATCGTGGGCTTTTACCTGCCACCCAATT
CTGGCGAGCGCGTGAGCTTCAAGATCACACTGCTGCTGGGCTATAGCGTG
TTTCTGATCATCGTGTCCGATACCCTGCCTGCAACAGCAATCGGAACCCC
ACTGATCGGCGTGTATTTCGTGGTGTGCATGGCCCTGCTGGTCATCAGCC
TGGCCGAGACAATCTTTATCGTGCGGCTGGTGCACAAGCAGGACCTGCAG
CAGCCTGTGCCAGCATGGCTGAGGCACCTGGTGCTGGAGAGGATCGCATG
GCTGCTGTGCCTGAGAGAGCAGTCCACATCTCAGAGGCCTCCAGCCACCT
CTCAGGCCACCAAGACAGACGATTGCTCTGCCATGGGCAATCACTGTAGC
CACATGGGCGGCCCCCAGGACTTTGAGAAGTCCCCTCGCGATCGGTGCTC
TCCACCTCCACCACCTAGGGAGGCCAGCCTGGCCGTGTGCGGCCTGCTGC
AGGAGCTGTCCTCTATCCGGCAGTTCCTGGAGAAGCGCGACGAGATCCGG
GAGGTGGCCAGAGATTGGCTGAGGGTGGGCAGCGTGCTGGATAAGCTGCT
GTTTCACATCTACCTGCTGGCAGTCCTGGCCTATTCTATTACCCTGGTCA
TGCTGTGGTCCATCTGGCAGTACGCC

In some cases, a nucleic acid sequence encoding a modified LGIC subunit described herein can encode an IPD from a GlyR. Examples of nucleic acid sequences encoding GlyR IPDs include, without limitation, a nucleic acid sequence set forth in SEQ ID NO:5. In some cases, a GlyR IPD can be a homolog, orthologue, or paralog of the human GlyR IPD set forth in SEQ ID NO:5. In some cases, a GlyR IPD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:25.

SEQ ID NO: 25
ATGGGTTATTATCTGATCCAAATGTATATCCCAAGCTTGCTTATAGTGAT
TTTGTCATGGATCTCCTTCTGGATTAATATGGACGCCGCTCCAGCTAGGG
TCGGACTGGGCATCACCACAGTGCTGACAATGACTACTCAGAGCTCAGGC
AGCCGAGCCAGCTTGCCCAAGGTTTCTTACGTGAAGGCCATCGATATCTG
GATGGCTGTCTGCCTTCTGTTTGTCTTCAGCGCACTGCTGGAATACGCCG
CTGTCAATTTTGTGTCTCGACAGCATAAAGAGCTGTTGCGGTTCAGAAGA
AAACGACGCCACCACAAAGAGGATGAGGCAGGAGAAGGACGCTTCAACTT
TAGCGCCTATGGTATGGGACCTGCTTGCCTCCAGGCTAAAGACGGAATTT
CCGTGAAGGGAGCCAACAATAGCAACACAACCAACCCACCCCCTGCTCCA
TCTAAGAGCCCGGAGGAAATGCGCAAACTCTTTATTCAGAGAGCGAAAAA
GATCGACAAAATCTCCCGGATCGGATTCCCCATGGCTTTCCTGATTTTCA
ACATGTTTTATTGGATCATCTACAAGATTGTGCGAAGGGAGGACGTACAC
AACCAGTAA

In some cases, a nucleic acid sequence encoding a modified LGIC subunit described herein can encode an IPD from a GABA receptor (e.g., GABAA-p, also referred to as GABAc). Examples of nucleic acid sequences encoding GABAA-P IPDs include, without limitation, a nucleic acid sequence set forth in SEQ ID NO:26. In some cases, a GABAA-P IPD can be have at least 75 percent sequence identity (e.g., at least 80%, at least 82%, at least 85%, at least 88%, at least 90%, at least 93%, at least 95%, at least 97% or at least 99% sequence identity) to SEQ ID NO:26.

SEQ ID NO: 26
CTTTTGCAAACTTACTTTCCAGCAACCCTCATGGTGATGCTTTCATGGGT
GTCCTTTTGGATCGACCGCCGAGCGGTCCCTGCACGGGTCCCCCTGGGGA
TTACGACGGTACTGACCATGAGCACCATAATCACTGGAGTCAATGCAAGC
ATGCCTAGAGTGTCTTACATAAAGGCCGTGGACATCTATCTGTGGGTTAG
TTTTGTGTTCGTATTCCTCTCCGTGCTGGAGTATGCAGCTGTGAACTATC
TGACAACAGTTCAAGAGCGGAAAGAGCAGAAGTTGAGGGAGAAGCTGCCA
TGCACTAGCGGACTGCCACCGCCCAGAACCGCTATGCTCGATGGTAACTA
TTCCGACGGCGAAGTTAATGACCTCGATAACTACATGCCTGAAAATGGCG
AAAAGCCCGACAGGATGATGGTCCAGCTGACACTGGCCTCAGAAAGGTCC
AGTCCACAGAGAAAGTCACAGCGATCCTCTTACGTCAGCATGCGCATCGA
TACACATGCCATCGACAAATACTCTCGCATTATCTTTCCGGCTGCTTACA
TATTGTTCAACCTTATCTATTGGAGCATTTTCAGTTGA

In calculating percent sequence identity, two sequences are aligned and the number of identical matches of amino acid residues between the two sequences is determined. The number of identical matches is divided by the length of the aligned region (i.e., the number of aligned nucleic acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. The alignment of two or more sequences to determine percent sequence identity can be performed using the computer program ClustalW and default parameters, which calculates the best match between a query and one or more subject sequences, and aligns them so that identities, similarities and differences can be determined. See, e.g., Chenna et al., 2003, Nucleic Acids Res., 31 (13): 3497-500.

A nucleic acid sequence encoding a modified LGIC described herein can include at least one modified nucleic acid such that the nucleic acid sequence can encode a LBD having at least one modified amino acid and/or an IPD having at least one modified amino acid. In some cases, a nucleic acid sequence encoding a modified LGIC described herein can include more than one (e.g., two, three, four, five, six, seven, eight, nine, ten, eleven, twelve, or more) modified nucleic acids. For example, a nucleic acid sequence can encode a modified LGIC subunit including a α7 LBD having at least 75 percent sequence identity to SEQ ID NO: 1, SEQ ID NO:2, SEQ ID NO:11, or SEQ ID NO:12, and an amino acid substitution at amino acid residue 27, 41, 77, 79, 131, 139, 141, 175, 210, 216, 217, and/or 219. Examples of nucleic acid codons at codon numbers 27, 41, 77, 79, 131, 139, 141, 175, 210, 216, 217, and/or 219 that can results amino acid substitutions at amino acid residues 27, 41, 77, 79, 131, 139, 141, 175, 210, 216, 217, and/or 219 can be as shown below.

TABLE 12
Codon usage resulting in LBD amino acid substitutions

| WT amino acid | |
|---|---|
| residue | Modified amino acid: Modified nucleic acid codon |
| R27 | D: GAC |
| E41 | R: AGG |
| W77 | F: TTC, Y: TAC |
| Q79 | A: GCA, G: GGC, S: TCG |
| Y115 | F: TTC |
| L131 | A: GCG, F: TTC, G: GGA, I: ATA, M: ATG, N: AAC, |
| | Q: CAA, V: GTG |
| Q139 | G: GGC, L: CTG |
| L141 | F: TTC or TTT |
| G175 | A: GCA, F: TTC, H: CAC, K: AAA, M: ATG, R: CGA, |
| | S: TCA, V: GTA |
| Y210 | F: TTT |
| P216 | I: ATC |
| Y217 | F: TTC |
| D219 | A: GCT |
| W298 | A: GCG |

For example, a nucleic acid sequence can encode a modified LGIC subunit including a GlyR IPD having at least 75 percent sequence identity to a sequence set forth in SEQ ID NO:5, and an amino acid substitution at amino acid residue 298 of an α7-GlyR IPD. Examples of nucleic acid codons at codon number 298 that can result in an amino acid substitution at amino acid residue 298 can be as shown below.

TABLE 13
Codon usage resulting in GlyR IPD amino acid substitutions

| WT amino acid | |
|---|---|
| residue | Modified amino acid: Modified nucleic acid codon |
| A298 | G: GGT |

* numbering is relative to the residue numbering set forth in SEQ ID NO: 7

For example, a nucleic acid sequence can encode a modified LGIC subunit including a GABAC IPD having at least 75 percent sequence identity to SEQ ID NO:9, and an amino acid substitution at amino acid residue 298 of an α7-GABAC chimeric receptor. Examples of nucleic acid codons at codon number 298 that can result in an amino acid substitution at amino acid residue 298 can be as shown below

TABLE 14
Codon usage resulting in GABAC IPD amino acid substitutions

| WT amino acid | |
|---|---|
| residue | Modified amino acid: Modified nucleic acid codon |
| W298* | A: GCG |

*numbering is relative to the residue numbering set forth in SEQ ID NO: 10

In some cases, a nucleic acid sequence encoding a modified LGIC described herein can encode a α7-5HT3 chimeric receptor as set forth in SEQ ID NO:6 (e.g., including a human α7 nAChR LBD (SEQ ID NO:1) and a murine 5HT3 IPD (SEQ ID NO:3) components). Examples of nucleic acid sequences encoding a α7-5HT3 chimeric receptor including a human α7 nAChR LBD and a murine 5HT3 IPD include, without limitation, a nucleic acid sequence set forth in SEQ ID NO:27.

SEQ ID NO: 27
ATGCGCTGTTCTCCAGGCGGCGTGTGGCTCGCCCTGGCTGCTTCCCTTCT
GCACGTTAGCCTGCAGGGTGAGTTCCAGCGCAAACTGTATAAGGAGCTTG
TTAAGAATTATAACCCCCTGGAGCGGCCGGTCGCAAATGATTCCCAGCCA
CTGACAGTGTACTTCAGCCTCTCCTTGCTGCAGATCATGGACGTGGATGA
AAAGAACCAGGTGCTGACCACTAATATTTGGTTGCAGATGTCCTGGACCG
ATCACTACTTGCAGTGGAATGTGAGCGAATACCCAGGTGTAAAGACTGTA
AGATTCCCTGACGGCCAAATCTGGAAACCAGATATCCTGCTGTACAACAG
CGCAGACGAAAGGTTTGATGCAACATTTCACACCAACGTGTTGGTCAATT
CTTCAGGCCACTGCCAGTACCTGCCCCCTGGAATCTTCAAGTCCTCATGC
TATATCGACGTCCGCTGGTTTCCCTTCGACGTCCAGCACTGCAAACTCAA
ATTCGGGAGCTGGAGCTACGGCGGATGGAGCCTGGATCTGCAAATGCAGG
AGGCTGACATCTCTGGTTACATCCCGAATGGGGAGTGGGACCTTGTGGGA
ATCCCCGGTAAAAGAAGCGAGCGATTTTATGAATGCTGCAAGGAACCCTA
CCCTGACGTAACATTCACAGTTATCATCAGAAGAAGGCCATTGTTCTACG
CCGTTAGTTTGTTGCTCCCCAGTATTTTTCTCATGGTCGTGGACATCGTG
GGATTTTGTCTCCCACCTGATAGCGGGGAGAGGGTCTCCTTTAAGATTAC
CTTGTTGCTCGGCTATTCTGTATTTCTGATCATCGTGTCCGATACCCTTC
CTGCCACAATCGGCACTCCGCTGATAGGAGTGTATTTCGTCGTGTGTATG
GCACTCCTGGTGATAAGTCTGGCGGAAACTATCTTCATTGTACGGCTGGT
ACATAAGCAGGACCTGCAAAGACCCGTGCCAGACTGGTTGCGACACCTTG
TGCTGGACAGAATTGCATGGATTCTGTGTCTTGGCGAGCAACCTATGGCC
CACCGGCCACCTGCAACCTTTCAAGCCAACAAGACAGACGATTGTAGTGG
GTCTGATCTGTTGCCTGCTATGGGGAATCACTGCTCCCATGTTGGGGGAC
CACAAGATTTGGAAAAGACCCCACGGGGGCGGGGATCACCCCTTCCTCCT
CCCCGAGAAGCCTCTCTCGCTGTCCGGGGGCTGCTCCAGGAACTGTCAAG
CATCCGACATTTTCTGGAGAAGCGGGACGAGATGAGGGAAGTCGCTAGAG
ACTGGCTGCGAGTGGGCTACGTCCTTGACAGGCTGCTGTTTCGGATCTAC
TTGCTGGCGGTGCTGGCTTATTCCATTACTCTGGTGACACTCTGGTCCAT
ATGGCACTACAGTTAG

In some cases, a nucleic acid sequence encoding a modified LGIC described herein can encode a α7-GlyR chimeric receptor as set forth in SEQ ID NO:7 (e.g., including a human α7 nAChR LBD (SEQ ID NO:2) and a human GlyR IPD (SEQ ID NO:5)). Examples of nucleic acid sequences encoding a α7-GlyR chimeric receptor including a human α7 nAChR LBD and a human GlyR IPD include, without limitation, a nucleic acid sequence set forth in SEQ ID NO:28.

SEQ ID NO: 28
ATGCGCTGTTCTCCAGGCGGCGTGTGGCTCGCCCTGGCTGCTTCCCTTCT
GCACGTTAGCCTGCAGGGTGAGTTCCAGCGCAAACTGTATAAGGAGCTTG
TTAAGAATTATAACCCCCTGGAGCGGCCGGTCGCAAATGATTCCCAGCCA
CTGACAGTGTACTTCAGCCTCTCCTTGCTGCAGATCATGGACGTGGATGA
AAAGAACCAGGTGCTGACCACTAATATTTGGTTGCAGATGTCCTGGACCG
ATCACTACTTGCAGTGGAATGTGAGCGAATACCCAGGTGTAAAGACTGTA
AGATTCCCTGACGGCCAAATCTGGAAACCAGATATCCTGCTGTACAACAG
CGCAGACGAAAGGTTTGATGCAACATTTCACACCAACGTGTTGGTCAATT
CTTCAGGCCACTGCCAGTACCTGCCCCCTGGAATCTTCAAGTCCTCATGC
TATATCGACGTCCGCTGGTTTCCCTTCGACGTCCAGCACTGCAAACTCAA
ATTCGGGAGCTGGAGCTACGGCGGATGGAGCCTGGATCTGCAAATGCAGG
AGGCTGACATCTCTGGTTACATCCCGAATGGGGAGTGGGACCTTGTGGGA
ATCCCCGGTAAAAGAAGCGAGCGATTTTATGAATGCTGCAAAGAGCCCTA
CCCAGATGTCACCTTCACAGTGACCATGCGGAGACGCATGGGTTATTATC
TGATCCAAATGTATATCCCAAGCTTGCTTATAGTGATTTTGTCATGGATC
TCCTTCTGGATTAATATGGACGCCGCTCCAGCTAGGGTCGGACTGGGCAT
CACCACAGTGCTGACAATGACTACTCAGAGCTCAGGCAGCCGAGCCAGCT
TGCCCAAGGTTTCTTACGTGAAGGCCATCGATATCTGGATGGCTGTCTGC
CTTCTGTTTGTCTTCAGCGCACTGCTGGAATACGCCGCTGTCAATTTTGT
GTCTCGACAGCATAAAGAGCTGTTGCGGTTCAGAAGAAAACGACGCCACC
ACAAAGAGGATGAGGCAGGAGAAGGACGCTTCAACTTTAGCGCCTATGGT
ATGGGACCTGCTTGCCTCCAGGCTAAAGACGGAATTTCCGTGAAGGGAGC
CAACAATAGCAACACAACCAACCCACCCCCTGCTCCATCTAAGAGCCCGG
AGGAAATGCGCAAACTCTTTATTCAGAGAGCGAAAAAGATCGACAAAATC
TCCCGGATCGGATTCCCCATGGCTTTCCTGATTTTCAACATGTTTTATTG
GATCATCTACAAGATTGTGCGAAGGGAGGACGTACACAACCAGTAA

In some cases, a nucleic acid sequence encoding a modified LGIC described herein can encode a α7-GABAC chimeric receptor set forth in SEQ ID NO:10 (e.g., including a human α7 nAChR LBD (SEQ ID NO:2) and a human GABAC IPD (SEQ ID NO:9)). Examples of nucleic acid sequences encoding a including a human α7 nAChR LBD (SEQ ID NO: 2) and a human GABAC IPD (SEQ ID NO:9) chimeric receptor including a human α7 nAChR LBD and a human GABAC IPD include, without limitation, a nucleic acid sequence set forth in SEQ ID NO:29.

SEQ ID NO: 29
ATGCGCTGTTCTCCAGGCGGCGTGTGGCTCGCCCTGGCTGCTTCCCTTCT
GCACGTTAGCCTGCAGGGTGAGTTCCAGCGCAAACTGTATAAGGAGCTTG
TTAAGAATTATAACCCCCTGGAGCGGCCGGTCGCAAATGATTCCCAGCCA
CTGACAGTGTACTTCAGCCTCTCCTTGCTGCAGATCATGGACGTGGATGA
AAAGAACCAGGTGCTGACCACTAATATTTGGTTGCAGATGTCCTGGACCG
ATCACTACTTGCAGTGGAATGTGAGCGAATACCCAGGTGTAAAGACTGTA
AGATTCCCTGACGGCCAAATCTGGAAACCAGATATCCTGCTGTACAACAG
CGCAGACGAAAGGTTTGATGCAACATTTCACACCAACGTGTTGGTCAATT
CTTCAGGCCACTGCCAGTACCTGCCCCCTGGAATCTTCAAGTCCTCATGC
TATATCGACGTCCGCTGGTTTCCCTTCGACGTCCAGCACTGCAAACTCAA
ATTCGGGAGCTGGAGCTACGGCGGATGGAGCCTGGATCTGCAAATGCAGG
AGGCTGACATCTCTGGTTACATCCCGAATGGGGAGTGGGACCTTGTGGGA
ATCCCCGGTAAAAGAAGCGAGCGATTTTATGAATGCTGCAAAGAGCCCTA
CCCAGATGTCACCTTCACAGTGACCATGCGGAGACGCACACTGTATTACC
TTTTGCAAACTTACTTTCCAGCAACCCTCATGGTGATGCTTTCATGGGTG
TCCTTTTGGATCGACCGCCGAGCGGTCCCTGCACGGGTCCCCCTGGGGAT
TACGACGGTACTGACCATGAGCACCATAATCACTGGAGTCAATGCAAGCA
TGCCTAGAGTGTCTTACATAAAGGCCGTGGACATCTATCTGTGGGTTAGT
TTTGTGTTCGTATTCCTCTCCGTGCTGGAGTATGCAGCTGTGAACTATCT
GACAACAGTTCAAGAGCGGAAAGAGCAGAAGTTGAGGGAGAAGCTGCCAT
GCACTAGCGGACTGCCACCGCCCAGAACCGCTATGCTCGATGGTAACTAT
TCCGACGGCGAAGTTAATGACCTCGATAACTACATGCCTGAAAATGGCGA
AAAGCCCGACAGGATGATGGTCCAGCTGACACTGGCCTCAGAAAGGTCCA
GTCCACAGAGAAAGTCACAGCGATCCTCTTACGTCAGCATGCGCATCGAT
ACACATGCCATCGACAAATACTCTCGCATTATCTTTCCGGCTGCTTACAT
ATTGTTCAACCTTATCTATTGGAGCATTTTCAGTTGA

In some cases, a nucleic acid sequence encoding a modified LGIC described herein can include a nucleic acid sequence encoding one or more additional polypeptide sequences (e.g., a transport sequence such as an export sequence and/or a signal sequence, or a targeting sequence). Examples of nucleic acid sequences encoding export sequences include, without limitation, a nucleic acid sequence encoding an ER export sequence (e.g., TTTTGCTATGAAAACGAAGTC; SEQ ID NO:30). Examples of nucleic acid sequences encoding signal sequences include, without limitation, a nucleic acid sequence encoding a CHRNB4 signal sequence (e.g., ATGAGAAGGGCCCCATCCCTGGTATTGTTTTTTTTGGTAGCTTTGTGCGGGAGGGG GAACTGC; SEQ ID NO:31). Examples of nucleic acid sequences encoding targeting sequences include, without limitation, a nucleic acid sequence encoding a KCNB1 somatic targeting sequence (e.g., CAAAGCCAACCTATCCTTAACACTAAAGAGAGCGCCGCTCAATCCAAACCCAAAG AAGAGTTGGAAATGGAGTCTATACCTTCACCTGTTGCACCTCTCCCTACTAGGACC GAAGGCGTGATTGACATGCGCTCTATGTCTAGTATAGATAGCTTTATATCCTGCGCC ACAGACTTTCCCGAAGCCACTAGGTTC; SEQ ID NO:32).

In some cases, a nucleic acid encoding a modified LGIC subunit described herein can be linked (e.g., operably linked) to one or more regulatory elements. For example, nucleic acids encoding modified LGIC subunits described herein can be operably linked to any appropriate promoter. A promoter can be a native (i.e., minimal) promoter or a composite promoter. A promoter can be a ubiquitous (i.e., constitutive) promoter or a regulated promoter (e.g., inducible, tissue specific, cell-type specific (e.g., neuron specific, muscle specific, glial specific), and neural subtype-specific). Examples of promoters that can be used to drive expression of nucleic acids encoding modified LGIC subunits described herein include, without limitation, synapsin (SYN), CAMKII, CMV, CAG, enolase, TRPVl, POMC, NPY, AGRP, MCH, and Orexin promoters. In some cases, a nucleic acid encoding a modified LGIC subunit described herein can be operably linked to a neuron specific promoter.

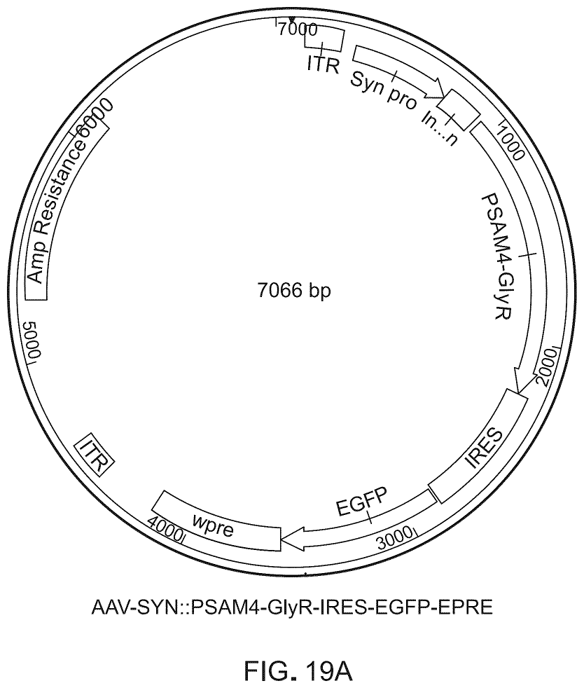

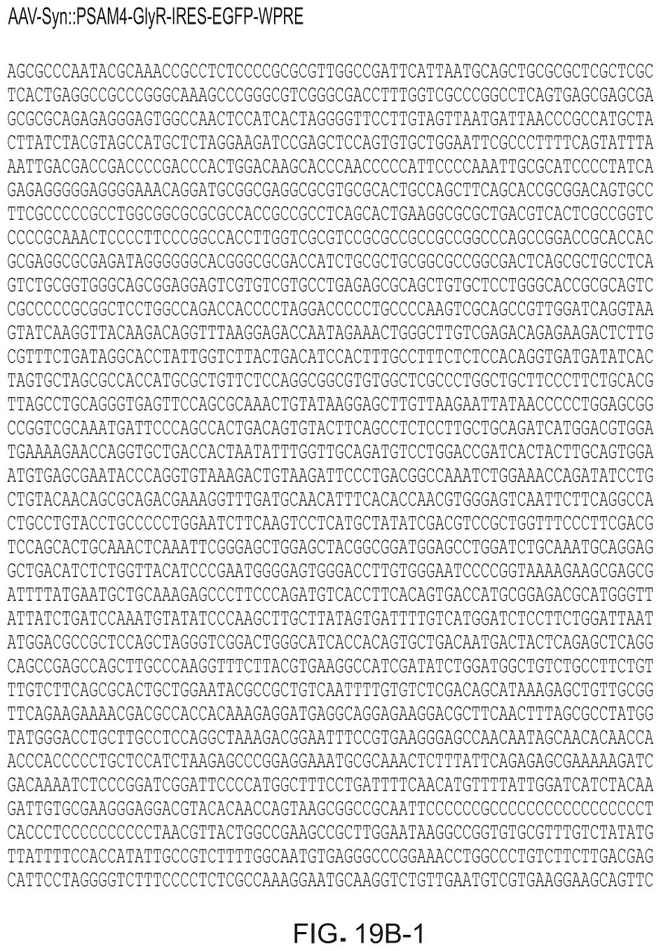

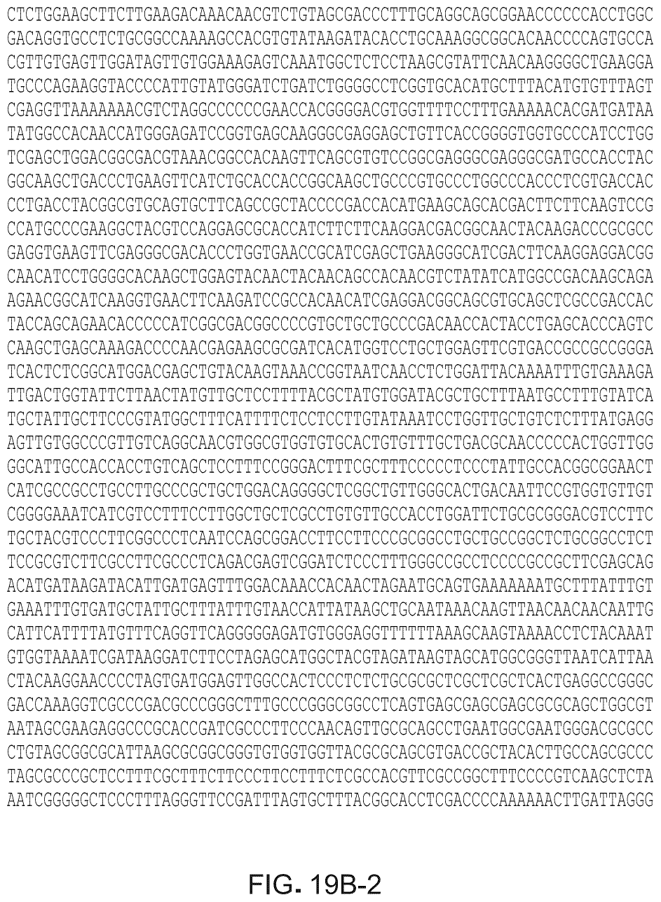

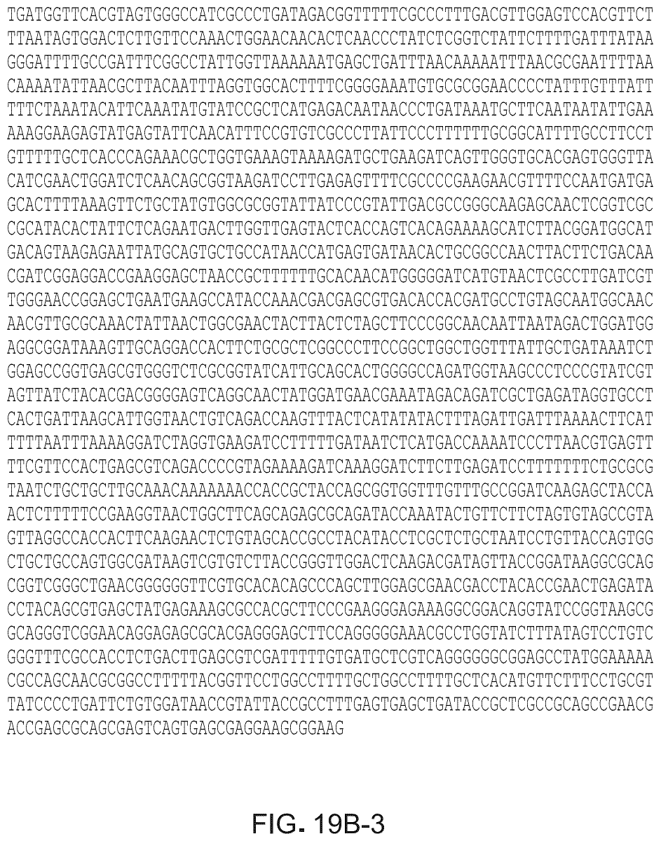

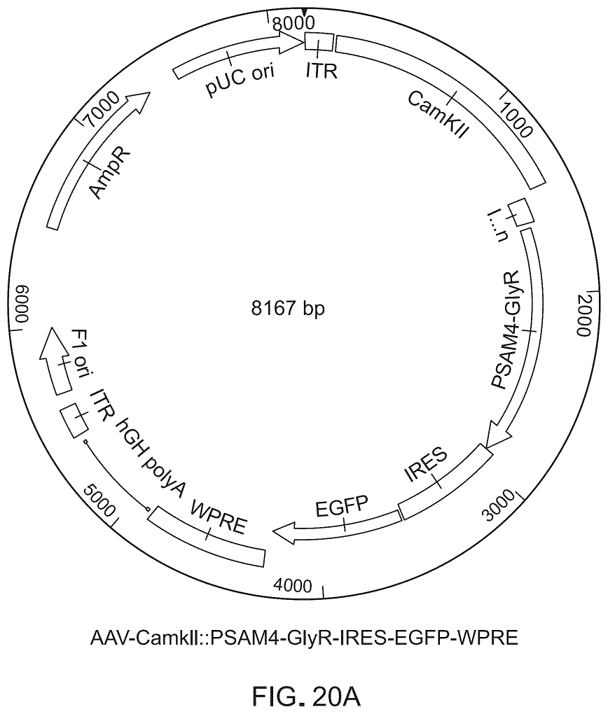

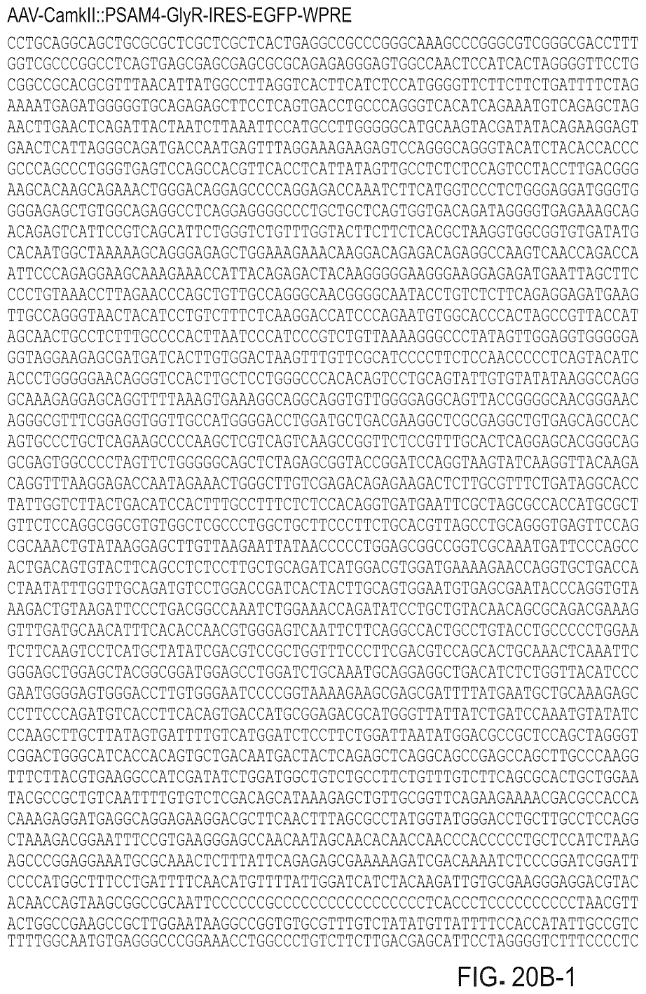

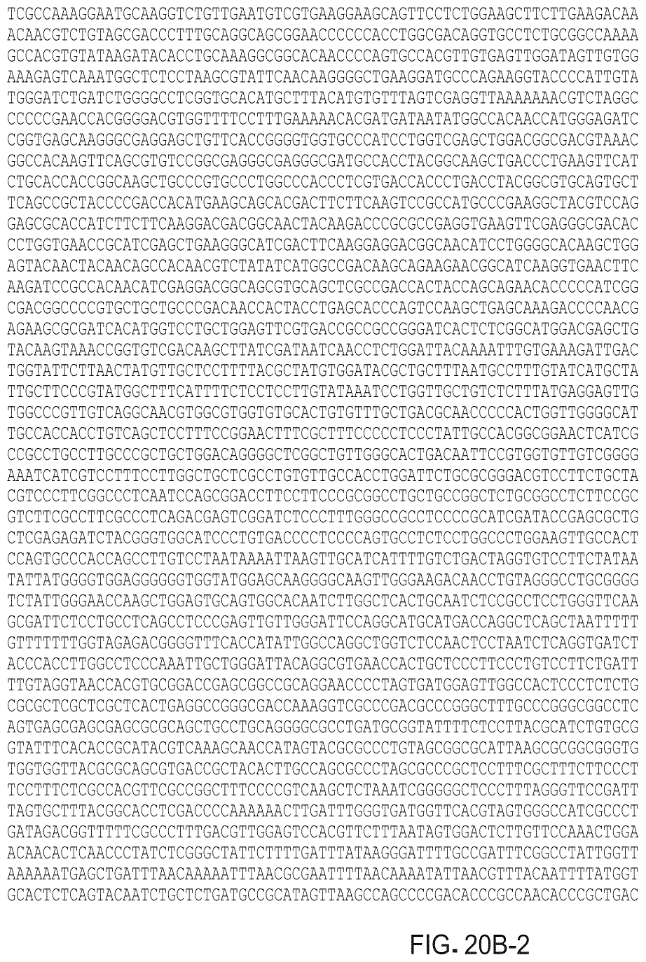

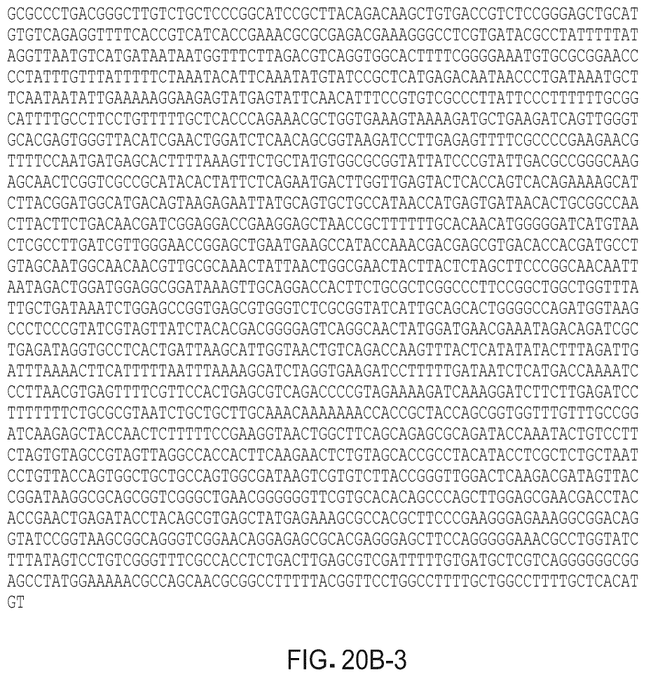

In cases where nucleic acids encoding modified LGIC subunits described herein are present in a construct, the construct can be any appropriate construct. A construct can be a nucleic acid (e.g., DNA, RNA, or a combination thereof) construct. Examples of constructs include, without limitation, plasmids, non-viral vectors, viral vectors (e.g., adeno-associated virus vectors, herpes simplex virus vectors, or lentivirus vectors). In some cases, a construct including a nucleic acid encoding a modified LGIC subunit described herein can express the modified LGIC subunit. In some cases, a construct can include an internal ribosome entry site (IRES; e.g., a bicistronic IRES). In some cases, a construct can include a nucleic acid sequence encoding a detectable marker (e.g., a fluorescent polypeptide such as a green fluorescent polypeptide (GFP; e.g., an enhanced GFP (EGFP))). In some cases, a construct can include a nucleic acid sequence providing the construct with a selectable marker (e.g., an antibiotic resistance marker such as ampicillin resistance). An exemplary plasmid map and nucleic acid sequence of a construct including a nucleic acid sequence encoding modified LGIC subunit described herein are shown in FIG. 19. Another exemplary plasmid map and nucleic acid sequence of a construct including a nucleic acid sequence encoding modified LGIC subunit described herein are shown in FIG. 20.

This document also provides cells (e.g., mammalian cells) having a modified LGIC described herein. Mammalian cells having a modified LGIC described herein can be obtained by any appropriate method. In some cases, a pre-assembled modified LGIC can be provided to the cell. In some cases, a nucleic acid encoding a modified LGIC subunit described herein can be provided to the cell under conditions in which a modified LGIC subunit is translated and under conditions in which multiple (e.g., three, four, five, six, or more) modified LGIC subunits can assemble into a modified LGIC described herein.

### LGIC Ligands

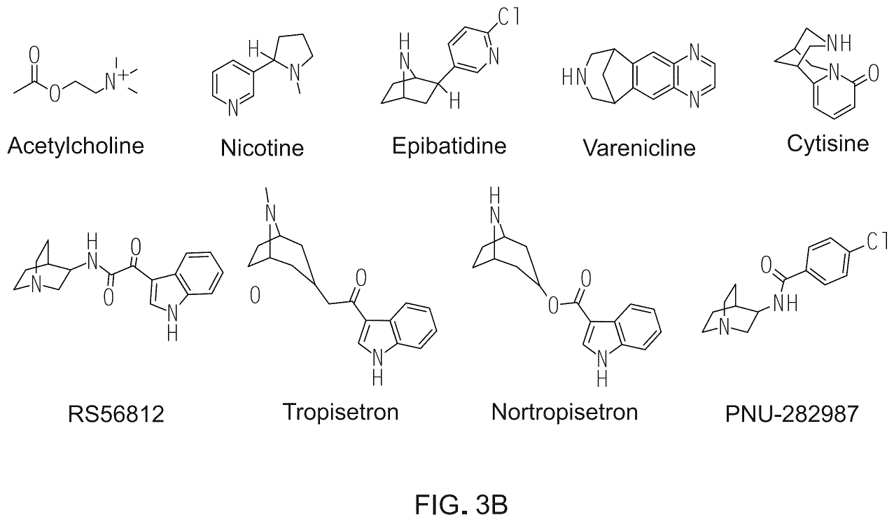

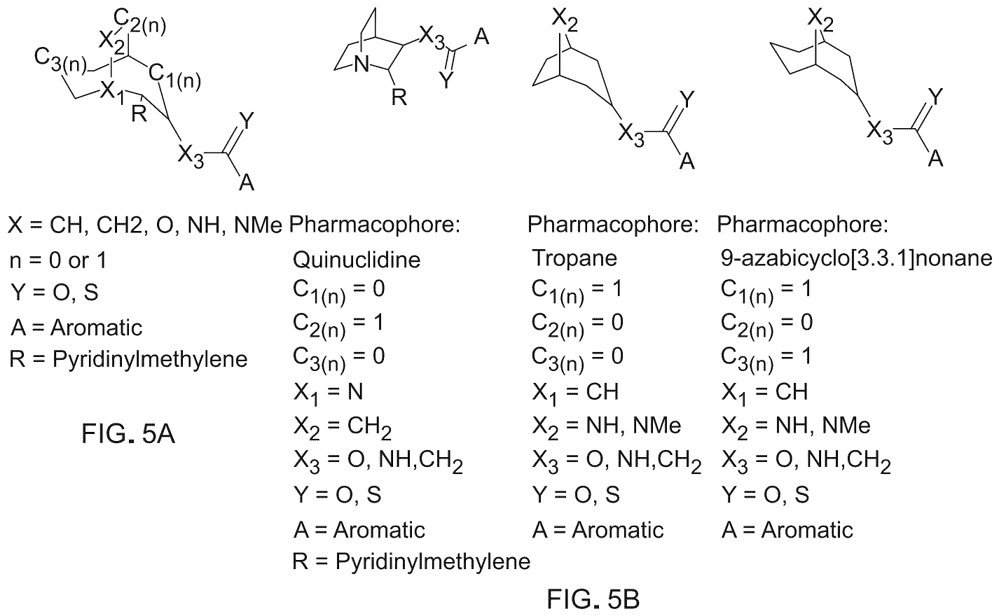

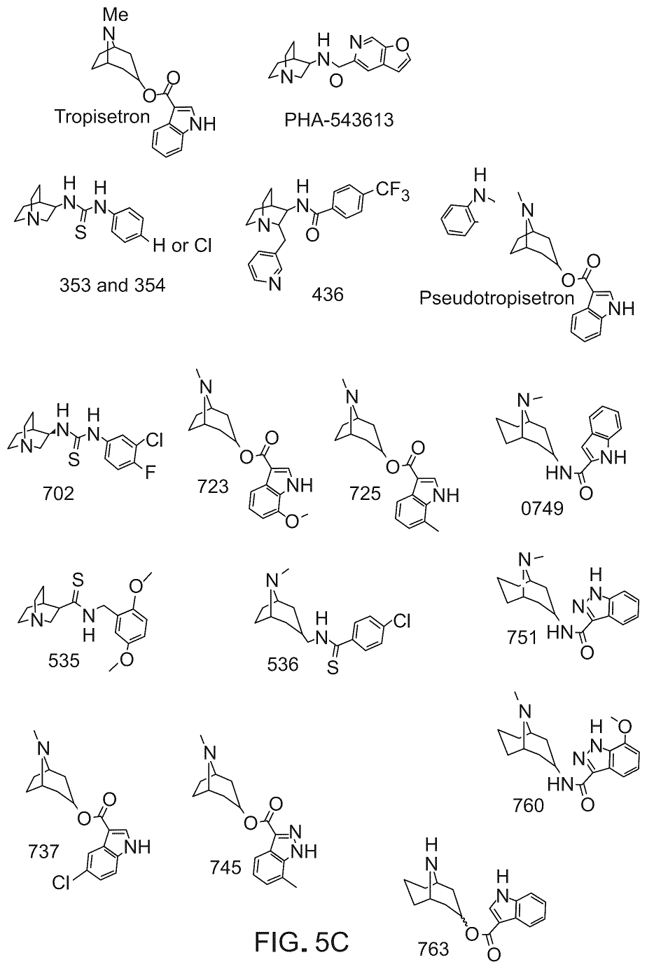

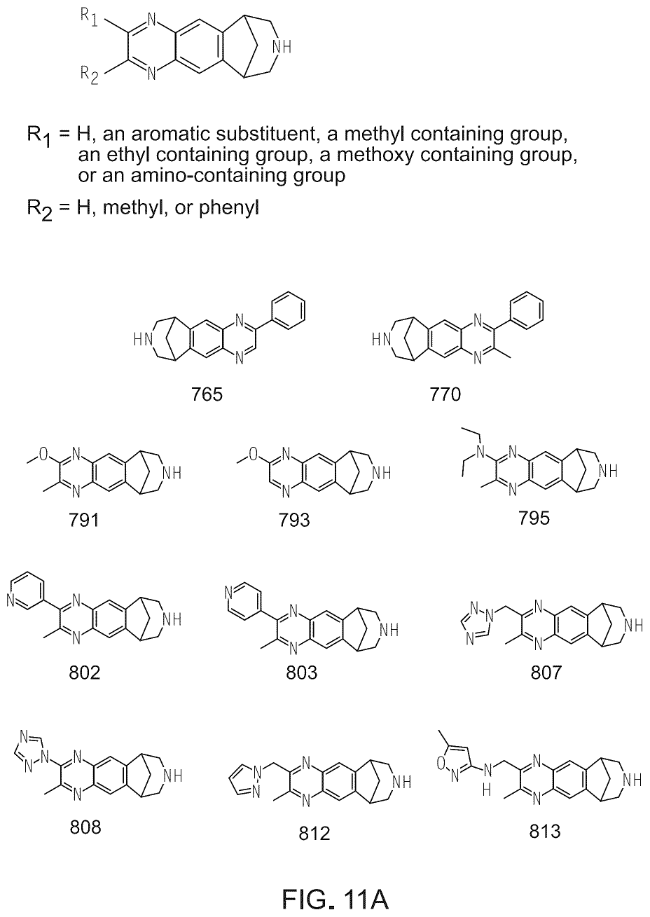

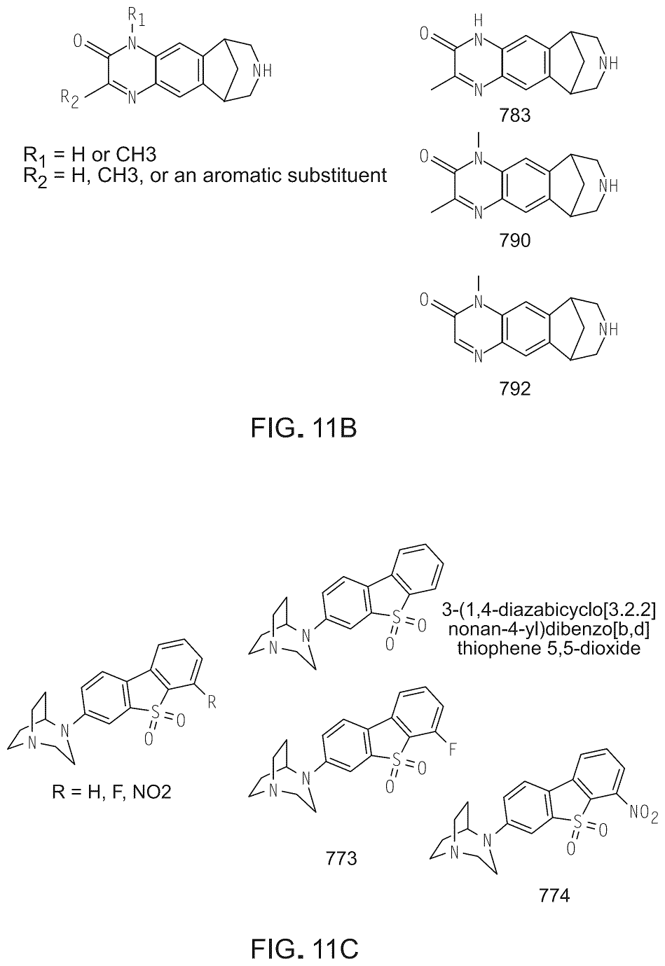

This document also provides LGIC ligands that can bind to and activate modified LGICs described herein. LGIC ligands can also be referred to as pharmacologically selective effector molecules (PSEMs). A LGIC ligand that can bind to and activate modified LGICs described herein can be exogenous or endogenous. A LGIC ligand that can bind to and activate modified LGICs described herein can be naturally occurring or synthetic. A LGIC ligand that can bind to and activate modified LGICs described herein can be canonical or non-canonical. A LGIC ligand that can bind to and activate modified LGICs described herein can be an agonist or an antagonist. In some cases, an LGIC ligand is an exogenous LGIC agonist. Examples of LGIC ligands include, without limitation, ACh, nicotine, epibatatine, cytisine, RS56812, tropisetron, nortropisetron, PNU-282987, PHA-543613, compound 0353, compound 0354, compound 0436, compound 0676, compound 702, compound 723, compound 725, granisetron, ivermectin, mequitazine, promazine, varenicline, compound 765, compound 770, 3-(1,4-diazabicyclo[3.2.2]nonan-4-yl)dibenzo[b,d]thiophene 5,5-dioxide, compound 773, and compound 774 (see, e.g., FIG. 3B, FIG. 5C, FIG. 11A, FIG. 11B, and FIG. 11C).

A LGIC ligand that can bind to and activate modified LGICs described herein can have selective binding (e.g., enhanced binding or increased potency) for a modified LGIC described herein (e.g., relative to an unmodified LGIC). In some cases, a LGIC ligand that can bind to and activate modified LGICs described herein does not bind to and activate endogenous receptors (e.g., endogenous LGICs). A LGIC ligand that selectively binds to and activates a modified LGIC (e.g., a modified LGIC having at least one amino acid modification that confers pharmacological selectivity to the modified LGIC) described herein over an unmodified LGIC ligand can also be described as having enhanced potency for a modified LGIC. In some cases, a modified LGIC subunit described herein that selectively binds an exogenous LGIC ligand can have at least 5 fold (e.g., at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 95 fold, at least 100 fold, at least 125 fold, at least 150 fold, at least 200 fold, at least 250 fold, or at least 300 fold) enhanced potency for a modified LGIC. For example, a LGIC ligand that selectively binds to and activates a modified LGIC can have about 10 fold to about 300 fold (e.g., about 10 fold to about 250 fold, about 10 fold to about 200 fold, about 10 fold to about 150 fold, about 10 fold to about 100 fold, about 25 fold to about 300 fold, about 50 fold to about 300 fold, about 100 fold to about 300 fold, about 200 fold to about 300 fold, about 25 fold to about 250 fold, about 50 fold to about 200 fold, or about 100 fold to about 150 fold) enhanced potency for a modified LGIC. In some cases, a LGIC ligand that binds to and activates a modified LGIC described herein can have a ligand potency of less than 25 nM (e.g., less than 22 nM, less than 20 nM, less than 17 nM, less than 15 nM, less than 13 nM, less than 12 nM, less than 11 nM, less than 10 nM, less than 5 nM, less, than 2 nM, or less than 1 nM). For example, a LGIC ligand that binds to and activates a modified LGIC described herein can have a ligand potency of less than 15 nM. In some cases, a LGIC ligand can have an EC50 of less than 25 nM (e.g., less than 22 nM, less than 20 nM, less than 17 nM, less than 15 nM, less than 13 nM, less than 12 nM, less than 11 nM, or less than 10 nM) for a modified LGIC subunit described herein. For example, a LGIC ligand (e.g., tropisetron) can have an EC50 of about 11 nM for a modified LGIC subunit described herein (e.g., α7^{Q79G}-GlyR^{A298G}). For example, a LGIC ligand (e.g., nortropisetron) can have an EC50 of about 13 nM for a modified LGIC subunit described herein (e.g., α7^{Q79G,Y115F}-GlyR^{A298G}). In some cases, a LGIC ligand can have an EC50 of greater than 20 μM (e.g., greater than 22 μM, greater than 25 μM, greater than 35 μM, greater than 50, greater than 65 μM, greater than 80 M, or greater than 100 μM) for a modified LGIC subunit described herein. For example, a LGIC ligand (e.g., ACh) can have an EC50 of greater than 100 μM for a modified LGIC subunit described herein (e.g., α7^{Q79G,Y115F}-GlyR^{A298G}).

In some aspects, a LGIC ligand can be a synthetic ligand that can bind to and activate modified LGICs described herein can be a quinuclidine, a tropane, a 9-azabicyclo[3.3.1]nonane, or a 2-phenyl-7,8,9,10-tetrahydro-6H-6,10-methanoazepino[4,5-g]quinoxaline.

A LGIC ligand that can be to and activate a modified LGIC described herein can have Formula I:

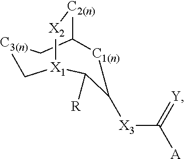

where X1 and X2 can independently be CH, CH2, O, NH, or NMe; each n can independently be 0 or 1; Y can be O or S; A can be an aromatic substituent; and R can be H or pyridinymethylene. Examples of aromatic substituents include, without limitation, 4-chloro-benzene, 1H-indole, 4-(trifluoromethyl)benzene, 4-chloro benzene, 2,5-dimethoxy benzene, 4-chloroaniline, aniline, 5-(trifluoromethyl)pyridin-2-yl, 6-(trifluoromethyl) nicotinic, and 4-chloro-benzene.

A LGIC ligand that can bind to and activate a modified LGIC described herein can be a quinuclidine. A quinuclidine can have the structure of Formula II:

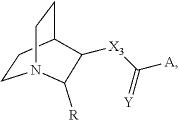

where X3 can be O, NH, or CH2; Y can be O or S; A can be an aromatic substituent; and R can be H or pyridinylmethylene. Examples of aromatic substituents include without limitation, 1H-indole, 4-(trifluoromethyl)benzene, 4-chloro benzene, 2,5-dimethoxy benzene, 4-(trifluoromethyl)benzene, 4-chloroaniline, aniline, 5-(trifluoromethyl)pyridin-2-yl, 6-(trifluoromethyl) nicotinic, 3-chloro-4-fluoro benzene, 4-chloro-benzene, and 1H-indole. Examples of quinuclidines include, without limitation, compounds PNU-282987, PHA-543613, 0456, 0434, 0436, 0354, 0353, 0295, 0296, and 0676 (see, e.g., FIG. 5C, Table 3, and Table 6).

A LGIC ligand that can bind to and activate a modified LGIC described herein can be a tropane. A tropane can have the structure of Formula III:

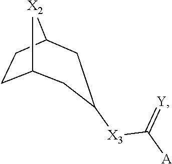

where X2 can be NH or NMe; X3 can be O, NH, or CH2; Y can be O or S; and A can be an aromatic substituent. Example of aromatic substituents include, without limitation, 1H-indole, 7-methoxy-1H-indole, 7-methyl-1H-indole, 5-chloro-1H-indole, and 1H-indazole. Examples of tropanes include, without limitation, tropisetron, pseudo-tropisetron, nortropisetron, compound 737, and compound 745 (see, e.g., FIG. 5C, Table 3, and Table 6).

A LGIC ligand that can bind to and activate a modified LGIC described herein can be a 9-azabicyclo[3.3.1]nonane. A 9-azabicyclo[3.3.1]nonane can have the structure of Formula IV:

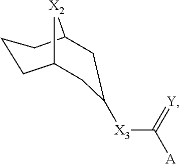

where X1 can be CH, X2 can be NH or NMe, X3 can be O, NH, or CH; Y can be O or S; and A can be an aromatic substituent. An example of an aromatic substituent is, without limitation, 4-chloro-benzene. Examples of 9-azabicyclo[3.3.1]nonanes include, without limitation, compound 0536, compound 0749, compound 0751, compound 0760, and compound 0763 (see, e.g., FIG. 5C, Table 3, and Table 6).

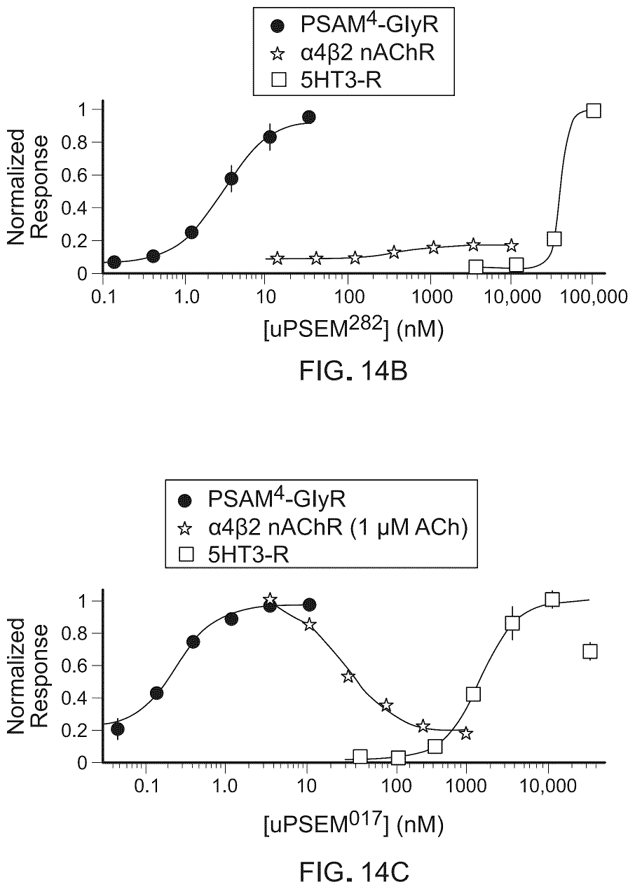

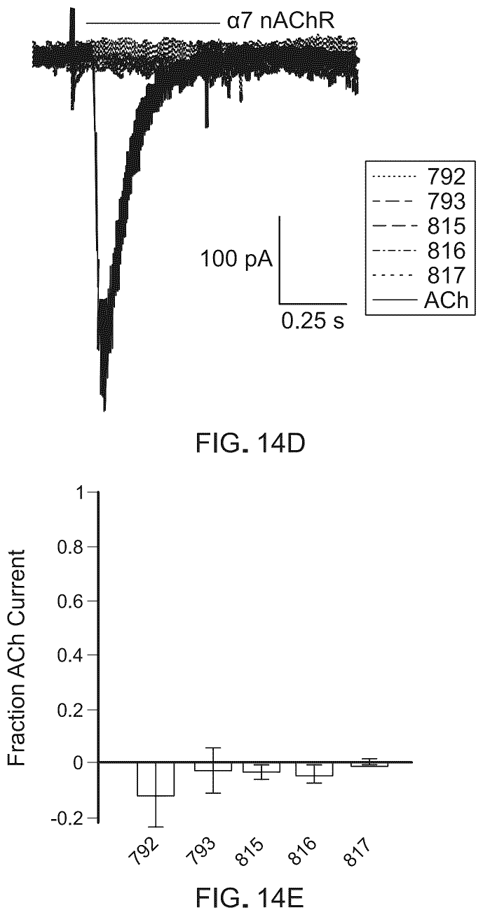

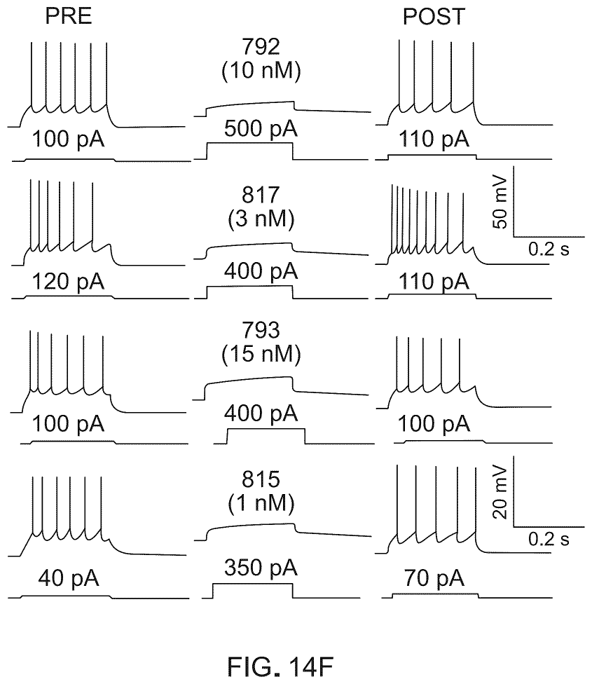

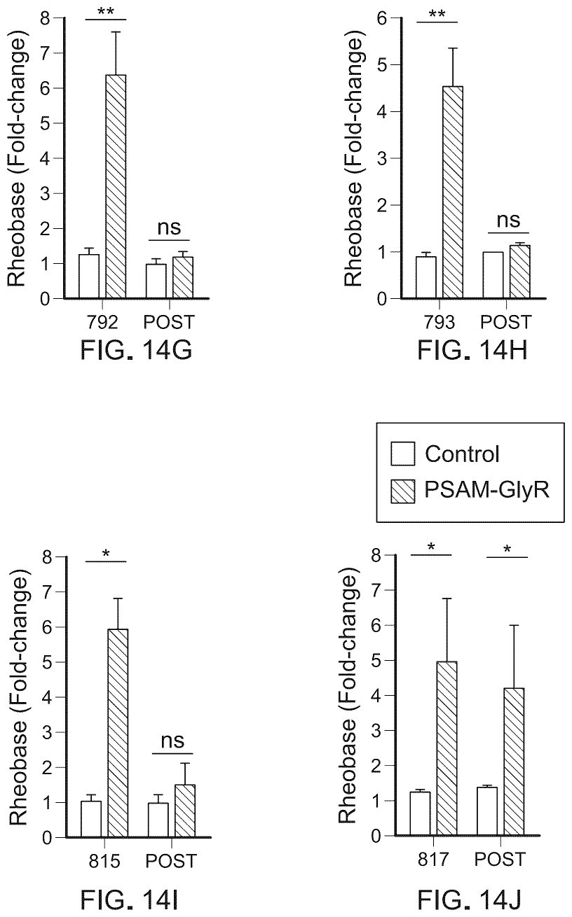

In some cases, a LGIC ligand can be an a 6,7,8,9-tetrahydro-6,10-methano-6H-pyrazino (2,3-h)benzazepine and can have a structure shown in Formula V:

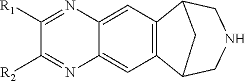

where R1=H, an aromatic substituent, a methyl containing group, an ethyl containing group, or other aliphatic groups, an alkoxy-containing group (e.g., methoxy-containing groups, ethoxy-containing groups, propoxy-containing groups, and isopropoxy-containing groups); or an amino-containing group; and R2=H, an aliphatic substituent (e.g., methyl), or an aromatic substituent (e.g., phenyl). Examples of R1 groups include, without limitation, phenyl, 2-toluyl, 3-pyridyl, 4-pyridyl, imidazole, pyrrole, pyrazole, triazole, isoxazole-3-amine, trifluoromethyl, methoxy, N,N-dimethylamino, and N,N-diethylamino. In some cases, R1 and R2 can be connected to form a ring. Examples of 6,7,8,9-tetrahydro-6,10-methano-6H-pyrazino (2,3-h)benzazepines include, without limitation, varenicline, compound 0765, compound 0770, compound 0780, compound 0782, compound 0785, compound 0788, compound 0782, compound 0789, compound 0791, compound 0793, compound 0794, compound 0795, compound 0798, compound 0799, compound 0800, compound 0801, compound 0802, compound 0803, compound 0804, compound 0805, compound 0807, compound 0808, compound 0812, compound 0813, compound 815, compound 816, and compound 817 (see, e.g., FIG. 11A, FIG. 14A, Table 3, Table 9, Table 10, and Table 11).

In some cases, a LGIC ligand can be a 2-(pyridin-3-yl)-1,5,6,7,8,9-hexahydro-5,9-methanoimidazo[4′,5′: 4,5]benzo[1,2-d]azepine:

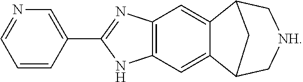 [SMILES: c1cncc(-c2nc3cc4c(cc3[nH]2)C2CNCC4C2)c1]

Examples of 2-(pyridin-3-yl)-1,5,6,7,8,9-hexahydro-5,9-methanoimidazo[4′,5′: 4,5]benzo[1,2-d]azepines include compound 0786 (see, e.g., Table 10 and Table 11).

In some cases, a LGIC ligand can be a 7,8,9,10-tetrahydro-1H-6,10-methanoazepino[4,5-g]quinoxalin-2 (6H)-one and can have a structure shown in formula VI:

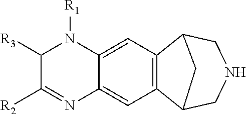

where R1=H or an aliphatic substituent (e.g., methyl); where R2=H, an aliphatic substituent (e.g., methyl), or an aromatic substituent; and where R3=O or S. Examples of 7,8,9,10-tetrahydro-1H-6,10-methanoazepino[4,5-g]quinoxalin-2 (6H)-ones include, without limitation, compound 0783, compound 0784, compound 0790, or compound 0792 (see, e.g., FIG. 11B, Table 10, and Table 11). For example, a LGIC can be a 7,8,9,10-tetrahydro-1H-6,10-methanoazepino[4,5-g]quinoxalin-2 (6H)-one having a structure of:

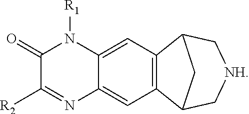

In some cases, a LGIC ligand can be a 1,4-diazabicyclo[3.2.2]nonane and can have a structure shown in Formula VII:

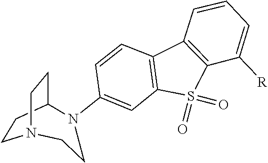

where R=H, F, NO₂. Examples of 1,4-diazabicyclo[3.2.2]nonanes include, without limitation, 3-(1,4-diazabicyclo[3.2.2]nonan-4-yl)dibenzo[b,d]thiophene 5,5-dioxide, compound 0773, and compound 0774 (see, e.g., FIG. 11C, Table 6, and Table 9).

### Methods of Using

This document also provides methods of using a modified LGIC described herein and a LGIC ligand that can bind to and activate the modified LGIC as described herein. A LGIC ligand that can bind to and activate the modified LGIC can be used to activate a modified LGIC with temporal and/or spatial control based on delivery of the ligand.

In some aspects, a modified LGIC described herein and a LGIC ligand that can bind to and activate the modified LGIC as described herein can be used to identify a ligand that selectively binds to a modified LGIC described herein. For example, such screening methods can include providing one or more candidate ligands to a modified LGIC described herein, and detecting binding between the candidate ligand and the modified LGIC.

Any appropriate method can be used to detect binding between a candidate ligand and the modified LGIC and any appropriate method can be used to detect activity of a modified LGIC. For example, the ability of a ligand to bind to and activate a modified LGIC can be measured by assays including, but not limited to, membrane potential (MP) assay (e.g., a fluorescence MP assay), radioactive binding assays, and/or voltage clamp measurement of peak currents and sustained currents.

In some aspects, a modified LGIC described herein and a LGIC ligand that can bind to and activate the modified LGIC as described herein can be used to treat a mammal having a channelopathy (e.g., a neural channelopathy or a muscle channelopathy). For example, a mammal having a channelopathy can be treated by administering a modified LGIC described herein, and then administering a LGIC ligand that can bind to and activate the modified LGIC. For example, a mammal having a channelopathy can be treated by administering a modified LGIC described herein (e.g., including at least one chimeric α7-GlyR LGIC subunit (SEQ ID NO:6) having a human α7 nAChR LBD (SEQ ID NO:2) with a R27D amino acid substitution, a E41R amino acid substitution, a Q79G amino acid substitution, and a Y115F amino acid substitution, and a human GlyR IPD (SEQ ID NO:5) with a A298G amino acid substitution), and then administering tropisetron. For example, a mammal having a channelopathy can be treated by administering a modified LGIC described herein including a modified human α7 nAChR LBD (e.g., SEQ ID NO:1, SEQ ID NO:2, SEQ ID NO:11, or SEQ ID NO:12) with an L131 amino acid substitution (e.g., L131G, L131A, L131M, or L131N) and, optionally, a Q79S amino acid substitution, a Q139L amino acid substitution, and/or a Y217F amino acid substitution, and then administering varenicline, tropisetron, and/or compound 765.

Any type of mammal can be treated using a modified LGIC described herein and a LGIC ligand that can bind to and activate the modified LGIC as described herein. For example, humans and other primates such as monkeys can be treated using a modified LGIC described herein and a LGIC ligand that can bind to and activate the modified LGIC as described herein. In some cases, dogs, cats, horses, cows, pigs, sheep, rabbits, mice, and rats can be treated using a modified LGIC described herein and a LGIC ligand that can bind to and activate the modified LGIC as described herein.

Any appropriate method can be used to identify a mammal having a channelopathy and/or a mammal at risk of developing a channelopathy. For example, genetic testing can be used to identify a mammal having a channelopathy and/or a mammal at risk of developing a channelopathy.

Once identified as having a channelopathy and/or a mammal at risk of developing a channelopathy, the mammal can be administered or instructed to self-administer a modified LGIC described herein, and then administered or instructed to self-administer a LGIC ligand that can bind to and activate the modified LGIC as described herein. A modified LGIC described herein and a LGIC ligand that can bind to and activate the modified LGIC as described herein can be administered together or can be administered separately.

When treating a mammal having a channelopathy and/or a mammal at risk of developing a channelopathy using the materials and methods described herein, the channelopathy can be any channelopathy. As used herein, a channelopathy can be any disease or disorder caused by aberrant ion channel function and/or aberrant ligand function, or which could be alleviated by modulated ion channel function and/or altered cellular ion flux (e.g., calcium ion flux). A channelopathy can be congenital or acquired. Examples of channelopathies include, without limitation, Bartter syndrome, Brugada syndrome, catecholaminergic polymorphic ventricular tachycardia (CPVT), congenital hyperinsulinism, cystic fibrosis, Dravet syndrome, episodic ataxia, erythromelalgia, generalized epilepsy (e.g., with febrile seizures), familial hemiplegic migraine, fibromyalgia, hyperkalemic periodic paralysis, hypokalemic periodic paralysis, Lambert-Eaton myasthenic syndrome, long QT syndrome (e.g., Romano-Ward syndrome), short QT syndrome, malignant hyperthermia, mucolipidosis type IV, myasthenia gravis, myotonia congenital, neuromyelitis optica, neuromyotonia, nonsyndromic deafness, paramyotonia congenital, retinitis pigmentosa, timothy syndrome, tinnitus, seizure, trigeminal neuralgia, and multiple sclerosis. Alternatively, or in addition, the materials and methods described herein can be used in other applications including, without limitation, pain treatment, cancer cell therapies, appetite control, spasticity treatment, muscle dystonia treatment, tremor treatment, and movement disorder treatment.

In some cases, a modified LGIC described herein and a LGIC ligand that can bind to and activate the modified LGIC as described herein can be used to modulate the activity of a cell. The activity of the cell that is modulated using a modified LGIC described herein and a LGIC ligand that can bind to and activate the modified LGIC as described herein can be any cellular activity. Examples of cellular activities include, without limitation, active transport (e.g., ion transport), passive transport, excitation, inhibition, ion flux (e.g., calcium ion flux), and exocytosis. The cellular activity can be increased or decreased. For example, a modified LGIC described herein and a LGIC ligand that can bind to and activate the modified LGIC as described herein can be used to modulate (e.g., increase) ion transport across the membrane of a cell. For example, a modified LGIC described herein and a LGIC ligand that can bind to and activate the modified LGIC as described herein can be used to modulate (e.g., increase) the excitability of a cell.

A modified LGIC described herein and a LGIC ligand that can bind to and activate the modified LGIC as described herein can be used to modulate the activity of any type of cell in a mammal. The cell can be a neuron, a glial cell, a myocyte, an immune cell (e.g., neutrophils, eosinophils, basophils, lymphocytes, and monocytes), an endocrine cell, or a stem cell (e.g., an embryonic stem cell). In some cases, the cell can be an excitable cell. The cell can be in vivo or ex vivo.

A modified LGIC described herein can be administered by any appropriate method. A modified LGIC can be administered as modified LGIC subunits or as pre-assembled modified LGICs. A modified LGIC can be administered as a nucleic acid encoding a modified LGIC. A modified LGIC can be administered as a nucleic acid encoding a modified LGIC subunit described herein. For example, a nucleic acid can be delivered as naked nucleic acid or using any appropriate vector (e.g., a recombinant vector). Vectors can be a DNA based vector, an RNA based, or combination thereof. Vectors can express a nucleic acid in dividing cells or non-dividing cells. Examples of recombinant vectors include, without limitation, plasmids, viral vectors (e.g., retroviral vectors, adenoviral vectors, adeno-associated viral vectors, and herpes simplex vectors), cosmids, and artificial chromosomes (e.g., yeast artificial chromosomes or bacterial artificial chromosomes). In some cases, a nucleic acid encoding a modified LGIC subunit described herein can be expressed by an adeno-associated viral vector.

A modified LGIC described herein can be detected (e.g., to confirm its presence in a cell) by any appropriate method. In some cases, an agent that selectively binds a modified LGIC can be used to detect the modified LGIC. Examples of agents that can be used to bind to a modified LGIC described herein include, without limitation, antibodies, proteins (e.g., bungarotoxin), and small molecule ligands (e.g., PET ligands). An agent that selectively binds a modified LGIC can include a detectable label (e.g., fluorescent labels, radioactive labels, positron emitting labels, and enzymatic labels). Methods to detect LGIC expression in a cell can include fluorescence imaging, autoradiography, functional MRI, PET, and SPECT.

A modified LGIC described herein and a LGIC ligand that can bind to and activate the modified LGIC as described herein can be administered to a mammal having a channelopathy and/or at risk of developing a channelopathy as a combination therapy with one or more additional agents/therapies used to treat a channelopathy. For example, a combination therapy used to treat a mammal having a channelopathy as described herein can include administering a modified LGIC described herein and a LGIC ligand that can bind to and activate the modified LGIC as described herein and treating with acetazolaminde, dichlorphenamide, mexilitine, glucose, calcium gluconate, L-DOPA, muscle stimulation, spinal stimulation, brain stimulation, and/or nerve stimulation.

In embodiments where a modified LGIC described herein and a LGIC ligand that can bind to and activate the modified LGIC as described herein are used in combination with additional agents/therapies used to treat a channelopathy, the one or more additional agents can be administered at the same time or independently. For example, a modified LGIC described herein and a LGIC ligand that can bind to and activate the modified LGIC as described herein first, and the one or more additional agents administered second, or vice versa. In embodiments where a modified LGIC described herein and a LGIC ligand that can bind to and activate the modified LGIC as described herein are used in combination with one or more additional therapies used to treat a channelopathy, the one or more additional therapies can be performed at the same time or independently of the administration of a modified LGIC described herein and a LGIC ligand that can bind to and activate the modified LGIC as described herein. For example, a modified LGIC described herein and a LGIC ligand that can bind to and activate the modified LGIC as described herein can be administered before, during, or after the one or more additional therapies are performed.

In some cases, a modified LGIC described herein and/or a LGIC ligand that can bind to and activate the modified LGIC as described herein can be formulated into a pharmaceutically acceptable composition for administration to a mammal having a channelopathy or at risk of developing a channelopathy. For example, a therapeutically effective amount of a modified LGIC described herein (e.g., a nucleic acid encoding a modified LGIC described herein) and/or a LGIC ligand that can bind to and activate the modified LGIC as described herein can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing a modified LGIC described herein and/or a LGIC ligand that can bind to and activate the modified LGIC as described herein can be designed for oral, parenteral (including subcutaneous, intracranial, intraarterial, intramuscular, intravenous, intracoronary, intradermal, or topical), or inhaled administration. When being administered orally, a pharmaceutical composition containing a therapeutically effective amount of a modified LGIC described herein (e.g., a nucleic acid encoding a modified LGIC described herein) and/or a LGIC ligand that can bind to and activate the modified LGIC as described herein can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. Compositions for inhalation can be delivered using, for example, an inhaler, a nebulizer, and/or a dry powder inhaler. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A pharmaceutically acceptable composition including a therapeutically effective amount of a modified LGIC described herein (e.g., a nucleic acid encoding a modified LGIC described herein) and/or a LGIC ligand that can bind to and activate the modified LGIC as described herein can be administered locally or systemically. In some cases, a composition containing a therapeutically effective amount of a modified LGIC described herein (e.g., a nucleic acid encoding a modified LGIC described herein) and/or a LGIC ligand that can bind to and activate the modified LGIC as described herein can be administered systemically by venous or oral administration to, or inhalation by a mammal (e.g., a human). In some cases, a composition containing a therapeutically effective amount of a modified LGIC described herein (e.g., a nucleic acid encoding a modified LGIC described herein) and/or a LGIC ligand that can bind to and activate the modified LGIC as described herein can be administered locally by percutaneous, subcutaneous, intramuscular, intracranial, or open surgical administration (e.g., injection) to a target tissue of a mammal (e.g., a human).

Effective doses can vary depending on the severity of the channelopathy, the route of administration, the age and general health condition of the subject, excipient usage, the possibility of co-usage with other therapeutic treatments such as use of other agents, and the judgment of the treating physician.

The frequency of administration can be any frequency that improves symptoms of a channelopathy without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing a therapeutically effective amount of a modified LGIC described herein (e.g., a nucleic acid encoding a modified LGIC described herein) and/or a LGIC ligand that can bind to and activate the modified LGIC as described herein can include rest periods. For example, a composition containing a therapeutically effective amount of a modified LGIC described herein (e.g., a nucleic acid encoding a modified LGIC described herein) and/or a LGIC ligand that can bind to and activate the modified LGIC as described herein can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the channelopathy may require an increase or decrease in administration frequency.

An effective duration for administering a composition containing a therapeutically effective amount of a modified LGIC described herein (e.g., a nucleic acid encoding a modified LGIC described herein) and/or a LGIC ligand that can bind to and activate the modified LGIC as described herein can be any duration that improves symptoms of a channelopathy without producing significant toxicity to the mammal. For example, the effective duration can vary from several days to several weeks, months, or years. In some cases, the effective duration for the treatment of a channelopathy can range in duration from about one month to about 10 years. Multiple factors can influence the actual effective duration used for a particular treatment. For example, an effective duration can vary with the frequency of administration, effective amount, use of multiple treatment agents, route of administration, and severity of the channelopathy being treated.

In certain instances, a course of treatment and the symptoms of the mammal being treated for a channelopathy can be monitored. Any appropriate method can be used to monitor the symptoms of a channelopathy.

The invention will be further described in the following examples, which do not limit the scope of the invention described in the claims.

### EXAMPLES

### Example 1: Potency-Enhancing Ligand Binding Domain Mutations

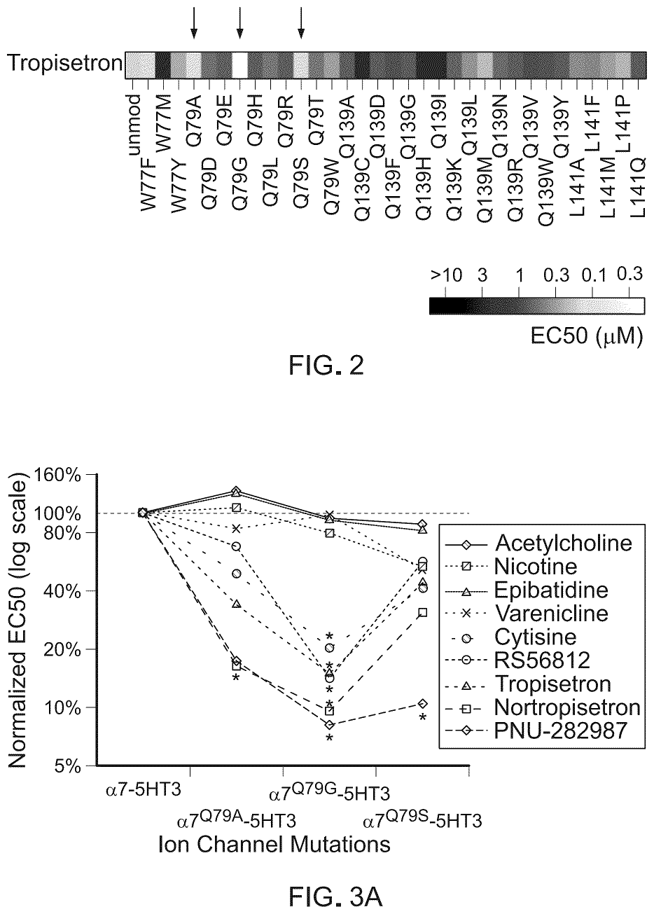

A screen was performed with a panel of 41 α7-5HT3 chimeric channels having mutant LBDs against a panel of 51 clinically used drugs with chemical similarity to nicotinic receptor agonists. Mutations were at residues highlighted in FIG. 1. The screen revealed mutations at Gln79 in the α7 nAChR LBD that enhanced potency for the known nAChR agonist tropisetron (FIG. 2). These mutations (Q79A, Q79G, Q79S) reduce the size of the amino acid side chain. Some mutant ion channel-ligand combinations gave up to 12-fold improvement in potency (Table 1, FIG. 3A-B). Canonical α7 nAChR agonists, ACh, nicotine, epibatidine, and the anti-smoking drug varenicline were not significantly affected by Q79A, Q79G, or Q79S mutations. However, a subset of α7 nAChR agonists showed enhanced potency with some of the mutations. Cytisine, RS56812, tropisetron, nortropisetron, and PNU-282987 showed significantly improved potency for α7^{Q79G}-5HT3. Additionally, nortropisetron and PNU-282987 showed a significantly enhanced potency for α7^{Q79A}-5HT3 and α7^{Q79S}-5HT3, respectively. In general, agonists based on a quinuclidine or tropane pharmacophore with a linked aromatic structure that interacts with the complementary binding face of the ligand binding domain showed improved potency with Gln79 substitution with the smaller amino acid residues Ala, Gly, or Ser. For most agonists, α7^{Q79G}-5HT3 was the most preferred mutant chimeric ion channel.

TABLE 1
Potency of nAChR agonists against chimeric cation channels mutated
at Gln79 in HEK cells. Mean EC50, SEM in parentheses (μM).

| Agonist | α7-5HT3 | | α7^{Q79A}-5HT3 | | α7^{Q79G}-5HT3 | | α7^{Q79S}-5HT3 | |
|---|---|---|---|---|---|---|---|---|
| Acetylcholine | 7.0 | (0.8) | 9.2 | (1.8) | 6.7 | (0.6) | 6.2 | (1.4) |
| Nicotine | 3.9 | (0.4) | 4.1 | (1.3) | 3.1 | (0.5) | 2.1 | (0.4) |
| Epibatidine | 0.053 | (0.006) | 0.067 | (0.022) | 0.050 | (0.008) | 0.044 | 0.006) |
| Varenicline | 0.92 | (0.16) | 0.76 | (0.21) | 0.91 | (0.12) | 0.47 | (0.07) |
| Cytisine | 8.2 | (0.3) | 4.0 | (0.9) | 1.7 | (0.2) | 4.4 | (1.0) |
| RS56812 | 10 | (1.8) | 6.8 | (1.9) | 1.4 | (0.2) | 5.7 | (0.8) |
| Tropisetron | 0.24 | (0.03) | 0.08 | (0.02) | 0.035 | (0.002) | 0.11 | (0.02) |
| Nortropisetron | 0.061 | (0.021) | 0.010 | (0.002) | 0.006 | (0.001) | 0.019 | (0.007) |
| PNU-282987 | 0.22 | (0.03) | 0.037 | (0.009) | 0.018 | (0.003) | 0.023 | (0.004) |

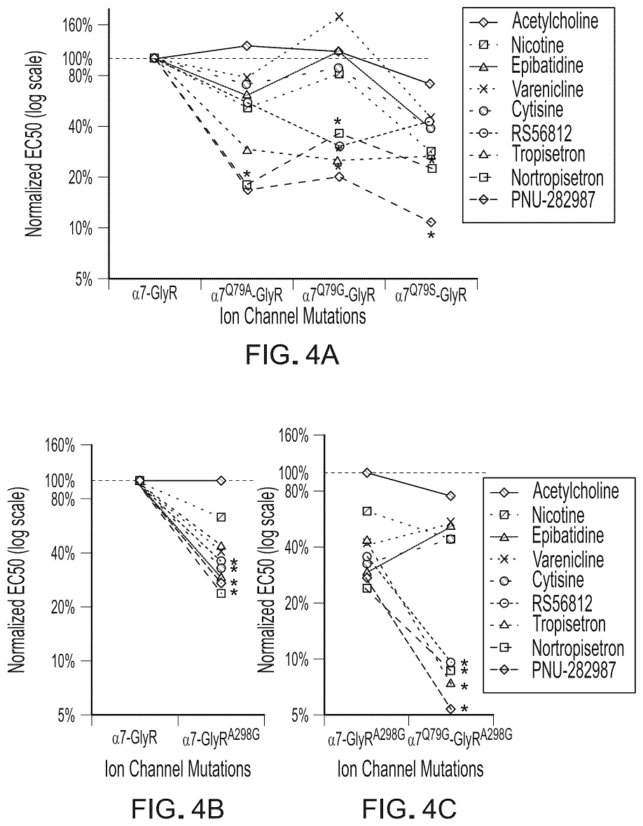

These mutated LBDs were used to generate α7-GlyR chimeric channels having enhanced potency for most of these ligands up to 6-fold (FIG. 4A). Like mutations of α7-5HT3, these mutations at Gln79 did not significantly affect potency of ACh, nicotine, epibatidine, varenicline, or cytisine. However, tropisetron, nortropisetron, and RS56812 showed significantly enhanced potency for α7^{Q79G}-GlyR. Similar to LBD mutations for α7-5HT3, nortropisetron had significantly enhanced potency for α7^{Q79A}-GlyR, and PNU-282987 showed significantly enhanced potency for α7^{Q79S}-GlyR. For most agonists, α7^{Q79G}-GlyR was the most preferred mutant chimeric ion channel.

Another relationship that was observed in the small molecule screen was that mutations at Trp77 conferred agonist activity for the drug granisetron at the α7\′77F-5HT3 (EC50: 1.2 μM), α7^{W77Y}-5HT3 (EC50: 1.1 μM), and α7^{W77F}-GlyR (EC50: 0.66 μM) receptors. Granisetron is a 5HT3 receptor antagonist granisetron, which does not activate α7-5HT3 or α7-GlyR.

These results show that mutation of Q79 (to A, G, or S) in the α7 nAChR LBD enhanced binding of known LGIC ligands to modified LGICs.

### Example 2: Potency Enhancing Ion Pore Domain Mutations

α7-GlyR channels having IPD mutations previously established in full length glycine receptor channels (T258S and A288G, GlyR numbering; equivalent to T268S and A298G for α7-GlyR numbering) were examined for enhanced potency for the allosteric agonist ivermectin. Channels having α7-GlyR^{T268S} were found to have substantial ligand-free open probability, which rendered them unsuitable for ligand-controlled manipulations of cells. Mutations at α7-GlyR^{A298G}, which were effective for enhancing ivermectin potency at the full length glycine receptor, led to modest change in open probability in the absence of the ligand; thus this channel was examined for activity against a panel of known agonists. For canonical agonists ACh, nicotine, and epibatidine, as well as for varenicline and tropisetron, the agonist potency was not significantly enhanced in α7-GlyR^{A298G}. A subset of α7 nAChR agonists did show up to a modest 4-fold increase in potency: RS56812, cytisine, PNU-282987, and nortropisetron were significantly more potent. Therefore, the effect of the IPD A298G mutation improved ligand potency, but depended on ligand structure and was not as effective as mutations in the LBD.

The Q79G mutation in the LBD and the A298G IPD mutation for α7-GlyR was examined (Table 2). The double mutant chimeric channel, α7^{Q79G}-GlyR^{A298G}, led to synergistic enhancement of potency showing up to 18-fold enhancement of potency relative to α7-GlyR to α7 nAChR agonists. The enhancement from this double mutant channel was greater than that from the individual mutations for agonists RS56812, tropisetron, nortropisetron, and PNU-282987. Further underscoring the unexpected structural sensitivity of this combination of mutations, multiple agonists, such as ACh, nicotine, epibatidine, varenicline, and cytisine were not significantly changed between α7-GlyR and α7^{Q79G}-GlyR^{A298G}. Therefore, combination of the LBD mutation Q79G with the IPD mutation A298G led to a synergistic effect where potency for some but not all nicotinic agonists was greatly increased by ˜10-20-fold.

TABLE 2
Potency of nAChR agonists against mutated chimeric chloride channels. Mean
EC50 and SEM in parentheses (μM) for agonist activity in HEK cells expressing chimeric
channels.

| | | | | | | α7^{Q79G}- |
|---|---|---|---|---|---|---|
| Agonist | α7 GlyR | α7^{Q79A}-GlyR | α7^{Q79G}-GlyR | α7^{Q79S}-GlyR | α7-GlyR^{A298G} | GlyR^{A298G} |
| Acetylcholine | 6.4 (1.2) | 7.6 (1.7) | 7.1 (1.2) | 4.5 (1.2) | 6.4 (1.8) | 4.8 (0.5) |
| Nicotine | 5.0 (1.8) | 2.6 (0.7) | 4.1 (0.3) | 1.4 (0.4) | 3.1 (1.8) | 2.2 (0.6) |
| Epibatidine | 0.062 (0.021) | 0.038 (0.005) | 0.069 (0.011) | 0.024 (0.003) | 0.018 (0.001) | 0.032 (0.007) |
| Varenicline | 0.62 (0.2) | 0.48 (0.08) | 1.1 (0.25) | 0.28 (0.06) | 0.25 (0.04) | 0.33 (0.08) |
| Cytisine | 6.4 (2.0) | 4.5 (0.6) | 5.6 (2.1) | 2.5 (0.7) | 2.1 (0.28) | 2.8 (1.0) |
| RS56812 | 6.5 (1.8) | 3.5 (0.5) | 2.0 (0.15) | 2.8 (0.5) | 2.3 (0.1) | 0.61 (0.14) |
| Tropisetron | 0.15 (0.045) | 0.044 (0.008) | 0.038 (0.003) | 0.040 (0.009) | 0.065 (0.026) | 0.011 (0.002) |
| Nortropisetron | 0.022 (0.007) | 0.004 (0.001) | 0.008 (0.003) | 0.005 (0.001) | 0.005 (0.001) | 0.002 (0.001) |
| PNU-282987 | 0.13 (0.038) | 0.022 (0.004) | 0.026 (0.005) | 0.014 (0.002) | 0.035 (0.005) | 0.007 (0.001) |

These results show that mutation of Q79 (to A, G, or S) in the α7 nAChR LBD and/or mutation of A298 (to G) in the GlyR IPD further enhanced selective binding of known LGIC ligands to modified LGICs.

### Example 3: Molecules Exhibiting Enhanced Potency

Based on the structure activity relationship of known agonists that showed enhanced potency with α7^{Q79G}-GlyR^{A298G}, a variety of synthetic molecules comprised of either quinuclidine, tropane, or 9-azabicyclo[3.3.1]nonane pharmacophores with one or more aromatic side chain substituents were tested. In addition, the known α7 nAChR agonist PHA-543613 (Walker et al 2006, Wishka et al 2006) was also tested and showed exceptional potency for α7^{Q79G}-GlyR^{A298G}. These molecules generally showed enhanced potency 10-fold to 100-fold (Table 3), indicating that, for these pharmacophores, a range of structural features were compatible with improved potency for α7^{Q79G}-GlyR^{A298G}.

These results show that modified LGICs can be activated by synthetic quinuclidine-containing and tropane-containing LGIC ligands.

TABLE 3
Potency of compounds against chimeric channels. Mean EC50 and SEM in parentheses (μM) for agonist activity in HEK cells
expressing chimeric channels. Partial refers to partial agonist activity.

| | | | | | | | | | | | | | α7^{Q79G}- |
|---|---|---|---|---|---|---|---|---|---|---|---|---|---|
| | | | | | | | | C-X | | | α7-5HT3 | α7-GlyR | GlyR^{A298G} |
| Compound | X₁ | X₂ | X₃ | Y | C₁ n | C₂ n | C₃ n | config | R | A | EC₅₀ (μM) | EC₅₀ (μM) | EC₅₀ (μM) |
| PNU-282987 | N | CH₂ | NH | O | 0 | 1 | 0 | R | H | 4-chloro-benzene | 0.22 | 0.13 | 0.007 |
| Tropisetron | C | NMe | O | O | 1 | 0 | 0 | Endo | H | 1H-indole | 0.24 | 0.15 | 0.011 |
| Pseudo- | C | NMe | O | O | 1 | 0 | 0 | Exo | H | 1H-indole | 2 | 0.7 | <0.2 |
| tropisetron | | | | | | | | | | | | | |
| Nortropisetron | C | NH | O | O | 1 | 0 | 0 | Endo | H | 1H-indole | 0.061 | 0.022 | 0.002 |
| PHA-543613 | N | CH₂ | NH | O | 0 | 1 | 0 | R | H | furo[2,3]pyridine | 0.046 | 0.039 | 0.004 |
| 0542 | C | NMe | NH | S | 1 | 0 | 0 | Endo | H | 1H-indole | 3.8 | 0.58 | 0.072 |
| 0026 | N | CH₂ | O | O | 0 | 1 | 0 | S | H | 4-(trifluoromethyl) | — | 13.7 | 1.43 |
| | | | | | | | | | | benzene | | | |
| 0456 | N | CH₂ | CH₂— | S | 0 | 1 | 0 | mix | H | 4-chloro benzene | — | 2.8 | 0.47 |
| | | | NH | | | | | | | | | | |
| 0434 | N | CH₂ | NH | O | 0 | 1 | 0 | mix | pyridin-3- | 2,5-dimethoxy | >10 | >10 | 0.19 |
| | | | | | | | | | ylmethyl | benzene | | | |
| 0436 | N | CH₂ | NH | O | 0 | 1 | 0 | mix | pyridin-3- | 4-(trifluoromethyl) | 0.84 | 0.31 | 0.006 |
| | | | | | | | | | ylmethyl | benzene | | | |
| 0354 | N | CH₂ | NH | S | 0 | 1 | 0 | R | H | 4-chloroaniline | 1.4 partial | 1.0 | 0.03 |
| 0353 | N | CH₂ | NH | O | 0 | 1 | 0 | S | H | aniline | 0.65 | 0.27 | 0.01 |
| 0295 | N | CH₂ | NH | O | 0 | 1 | 0 | S | H | 5-(trifluoromethyl) | >100 | >100 | 4.6 |
| | | | | | | | | | | pyridin-2-yl) | | | |
| 0296 | N | CH | NH | O | 0 | 1 | 0 | S | H | 6-(trifluoromethyl) | >100 | — | 0.45 |
| | | | | | | | | | | nicotinic | | | |
| 0536 | C | NMe | NH | S | 1 | 0 | 1 | Endo | H | 4-chloro-benzene | >33 | >100 | 9.1 |
| 0676 | N | CH₂ | NH | O | 0 | 1 | 0 | S | H | 1H-indole | 0.03 | 0.018 | 0.002 |

### Example 4: Mutations that Reduce Acetylcholine Responsiveness

The α7 nAChR has relatively low sensitivity to ACh compared to other nAChR isoforms, and potency enhancing mutations for tropane and quinuclidine ligands did not substantially alter the potency of acetylcholine at these channels. Thus, the chimeric channels were further modified to reduce acetylcholine responsiveness of these channels. Acetylcholine responsiveness was considerably reduced to more than 100 μM in some cases with additional LBD mutations Y115F and Q139G that that only modestly reduced the potency of some agonists for α7^{Q79G,Y115F}-5HT3, α7^{Q79G,Q139G}-5HT3, α7^{Q79G,Q139G}-GlyR^{A298}, α7^{Q79G,Y115F}-GlyR^{A298G} For example, α7^{Q79G,Y115F}-GlyR^{A298G} has an EC50 of 13 nM for nortropisetron and >100 μM for ACh (Table 4).

TABLE 4
Potency of nAChR agonists against mutated chimeric chloride channels with low
acetylcholine responsiveness. Mean EC50 and SEM in parentheses
(μM) for activity in HEK cells expressing chimeric channels.

| | α7^{Q79G,Y115F}- | | α7^{Q79G,Q139G}- | | α7^{Q79G,Y115F}- | | α7^{Q79G,Q139G}- | | α7^{R27D,E41R,Q79G,Y115F}- | |
|---|---|---|---|---|---|---|---|---|---|---|
| | 5HT3 | | 5HT3 | | GlyRA298G | | GlyRA298G | | GlyRA298G | |
| Acetylcholine | >100 | | 36 | (2) | >100 | | 73 | (27) | >100 | |
| Nicotine | 34 | (4) | 24 | (4) | 22 | (3) | 30 | (8) | 7.5 | (1.3) |
| Tropisetron | 0.10 | (0.12) | 0.31 | (0.06) | 0.086 | (0.043) | 0.26 | (0.04) | 0.035 | (0.021) |
| Nortropisetron | 0.028 | (0.005) | 0.047 | (0.013) | 0.013 | (0.001) | 0.031 | (0.006) | 0.003 | (0.001) |
| PNU-282987 | 0.35 | (0.07) | 0.16 | (0.04) | 0.22 | (0.04) | 0.18 | (0.04) | 0.066 | (0.010) |

These results show that Y115F and/or Q139G mutations in the α7 nAChR LBD reduced binding of the endogenous LGIC ligand Ach to the modified LGIC.

### Example 5: Mutations that Reduce Associations with Endogenous Receptor Subunits

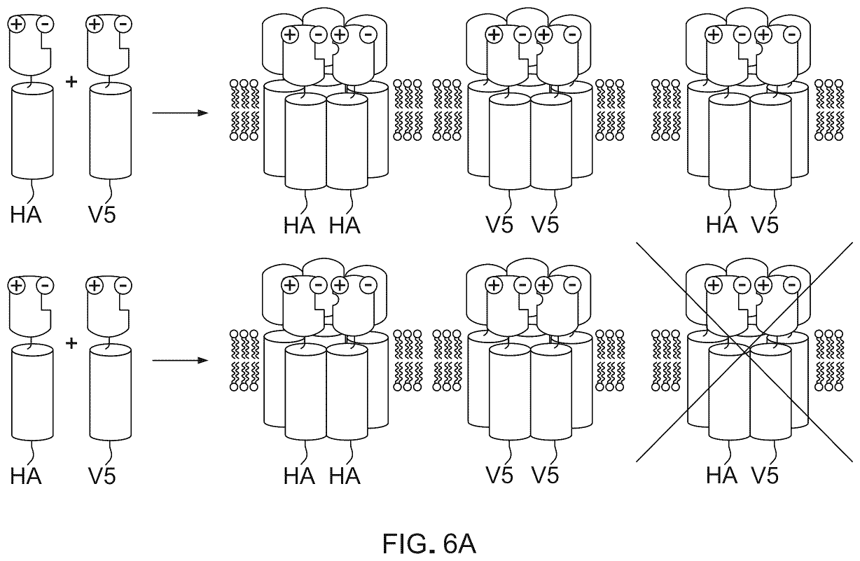

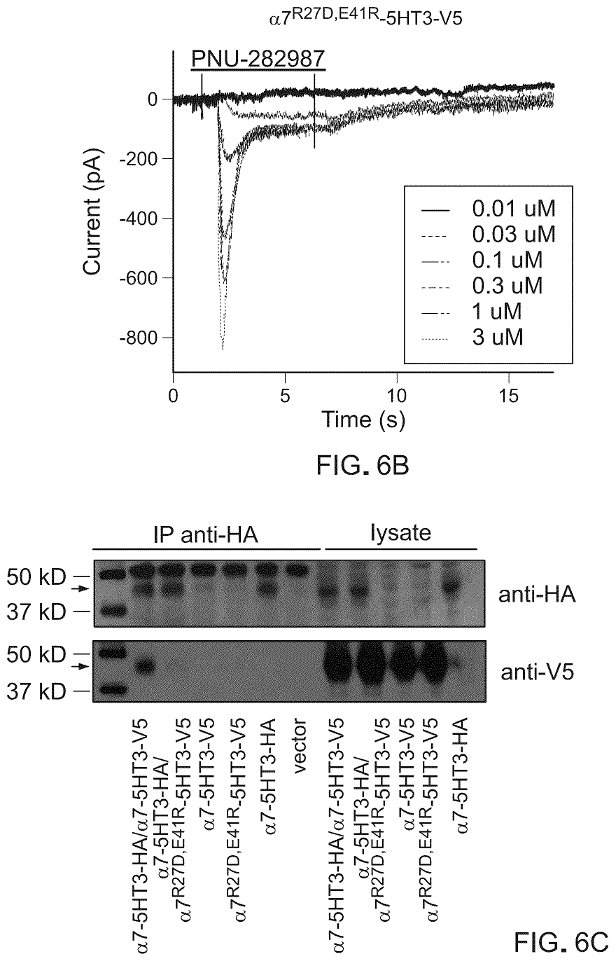

Assembly of α7 nAChRs is based on associations of five homomeric subunits through interactions between the LBDs (Celie et al 2004 Neuron 41:907-14). To minimize undesired associations with endogenous α7 nAChR subunits and/or unwanted associations of chimeric channels, potential inter-subunit salt bridges were identified by examining the crystal structure of the acetylcholine binding protein and identifying nearby inter-subunit residues with opposite charge that also have homologous ionic amino acids in the α7 nAChR receptor LBD. Charge reversal mutations (switching the acidic member of a potential salt bridge to a basic residue and its basic partner to an acidic residue) were designed to disrupt inter-subunit interactions with unmodified subunits but preserve interactions between the subunits with charge reversal mutations (FIG. 6A). Chimeric LGIC subunits having charge reversal mutations were able to assemble selectively with each other without interacting with unmodified channels, e.g. endogenous α7 nAChR. The double mutation of R27D, E41R in the α7 nAChR LBD resulted in functional channels (FIG. 6B). Co-expression of these charge reversal channels with α7-5HT3 channels having an unmodified sequence showed that the charge reversal subunits did not co-immunoprecipitate with unmodified channels (FIG. 6C). Combination with potency enhancing mutations and acetylcholine blocking mutations to give the chimeric channel α7^{R27D,E41R,Q79G,Y115F}-GlyR^{A298G} revealed that some agonists retained high potency for their cognate agonist (Table 4, right column).

These results show that R27D and E41R mutations in α7 nAChR LBD reduced association of the modified LGIC subunits with other modified and/or endogenous LGIC subunits.

### Example 6: LBD Mutations that Increase Ligand Potency

Mutations in Gly¹⁷⁵ and Pro²¹⁶ of the α7 nAChR LBD in α7-GlyR chimeric channels were tested. Mutation of Gly¹⁷⁵ to Lys (α7^{G175K}-GlyR) showed increased potency for ACh (5-fold) (Table 5). For α7^{G175K}-GlyR, it was also found that nicotine potency was enhanced 10-fold relative to the unmodified α7-GlyR chimeric channel (Table 5). Mutation of Pro²¹⁶ to Ile (α7^{P216I}-GlyR) did not substantially alter ACh potency (Table 5). However, α7^{P216I}-GlyR showed increased nicotine potency by >4-fold relative to unmodified α7-GlyR (Table 5). These potency enhancing mutations in α7^{G175K}-GlyR and α7^{P216I}-GlyR also affected potency of several other α7-GlyR agonists up to 30-fold (Table 5). For α7^{G175K}-GlyR, greater than 10-fold potency enhancement over α7-GlyR was seen for the clinically used drugs tropisetron, varenicline, cytisine, granisetron, and epibatidine. For α7^{P216I}-GlyR, potency enhancement was approximately 3-fold (Table 5).

TABLE 5
Agonist potency enhancement by G175K and P216I mutations at a7GlyR chimeric channels.
Units: μM. Parentheses: SEM.

| | | | | | | | | | | | | | | | α7GlyR | |
|---|---|---|---|---|---|---|---|---|---|---|---|---|---|---|---|---|
| | | | | | | | α7GlyR | | α7GlyR | | α7GlyR | | α7GlyR | | ^{W77F} | |
| | | | α7GlyR | | α7GlyR | | ^{Y115F} | | ^{G175K} | | ^{W77F} | | ^{Q79G} | | ^{Q79G} | |
| Compound | a7GlyR | | ^{G175K} | | ^{P216I} | | ^{G175K} | | ^{Y210F} | | ^{G175K} | | ^{G175K} | | ^{G175K} | |
| Acetyl- | 6.4 | (1.2) | 1.2 | (0.41) | 4.0 | (0.5) | 52 | (6.6) | 93 | (1.3) | 6.8 | (1.6) | 4.5 | (1.3) | 41 | (3.1) |
| choline | | | | | | | | | | | | | | | | |
| Nicotine | 5.0 | (1.8) | 0.5 | (0.25) | 1.4 | (0.1) | 4.1 | (1.4) | 6 | (0.5) | 1.3 | (0.4) | 1.1 | (0.1) | 2.6 | (0.7) |
| Epibatidine | 0.062 | (0.021) | 0.005 | (0.001) | 0.03 | (0.01) | 0.036 | (0.006) | 0.65 | (0.11) | 0.04 | (0) | 0.037 | (0.013) | 2.6 | (2.3) |
| Varenicline | 0.62 | (0.2) | 0.056 | (0.014) | 0.18 | (0.06) | 5.0 | (1.7) | 4.3 | (0.6) | 0.57 | (0.18) | 0.42 | (0.1) | 3.3 | (1.0) |
| Cytisine | 6.4 | (2.0) | 0.4 | (0.05) | 1.9 | (0.2) | 7.1 | (1.2) | >10 | | 1.5 | (0.6) | 2.5 | (1.1) | 6.9 | (1.2) |
| PNU- | 0.13 | (0.038) | 0.005 | (0.001) | 0.04 | (0.004) | 0.1 | (0.01) | 0.7 | (0.3) | 0.67 | (0.35) | 0.06 | (0.05) | 0.5 | (0.2) |
| 282987 | | | | | | | | | | | | | | | | |
| Tropisetron | 0.15 | (0.045) | 0.011 | (0.002) | 0.05 | (0.003) | 0.027 | (0.004) | 1.1 | (0.2) | 0.04 | (0.01) | 0.01 | (0.001) | 0.024 | (0.004) |
| Nortro- | 0.022 | (0.007) | 0.003 | (0.002) | 0.006 | (0.0004) | 0.007 | (0.001) | 0.28 | (0.09) | 0.004 | (0.001) | 0.0008 | (0.0001) | 0.0026 | (0.0004) |
| pisetron | | | | | | | | | | | | | | | | |
| PHA- | 0.03 | (0.01) | 0.001 | (0.0001) | 0.009 | (0.001) | 0.02 | (0.007) | 0.26 | (0.08) | 0.041 | (0.016) | 0.003 | (0.0004) | 0.12 | (0.04) |
| 543613 | | | | | | | | | | | | | | | | |

| | | | | | | | | | | | | | | |
|---|---|---|---|---|---|---|---|---|---|---|---|---|---|---|
| Granisetron | >100 | 3.3 | (0.1) | 6.1 | (0.9) | 1.6 | (0.6) | 1.4 | (0.1) | 0.18 | (0.02) | >100 | 1.6 | (0.4) |

| Ivermectin | | nd | | nd | | nd | | nd | nd | nd | nd |
|---|---|---|---|---|---|---|---|---|---|---|---|
| | | α7GlyR | | | | | | α7GlyR | α7GlyR | | |
| | | ^{W77F} | | α7GlyR | | α7GlyR | | ^{Q79G} | ^{Q79G} | α7GlyR | α7GlyR |
| | | ^{Q79G} | | ^{W77F} | | ^{Q79G} | | ^{Y115F} | ^{Y115F} | ^{Y115F} | ^{Q79G} |
| | | ^{Y115F} | | ^{G175K} | | ^{G175K} | | ^{G175K} | ^{G175K} | ^{G175K} | ^{Q139L} |
| | Compound | ^{G175K} | | ^{Y210F} | | ^{Y115F} | | ^{Y210F} | ^{K322L} | ^{L141F} | ^{G175K} |
| | Acetyl- | 143 | (13) | 80 | (31) | 98 | (10) | >1000 | >200 | 58 | 53 |
| | choline | | | | | | | | | | |

| Nicotine | 6.1 | (2.0) | 4.2 | 13 | (0.2) | >100 | 14.5 | 3 | 5.8 |
|---|---|---|---|---|---|---|---|---|---|
| Epibatidine | 0.33 | | 0.38 | 0.22 | (0.015) | >10 | 0.27 | 0.144 | 0.144 |
| Varenicline | >10 | | >9 | >10 | | >30 | >30 | >8.1 | 0.96 |
| Cytisine | 4.02 | | 5.1 | >10 | | >30 | >30 | 4.74 | 3.24 |
| PNU- | >1 | | >40 | 0.08 | (0.01) | >1 | 0.018 | 0.51 | 0.05 |
| 282987 | | | | | | | | | |
| Tropisetron | 0.1 | (0.04) | >1 | 0.027 | (0.002) | 0.717 | 0.066 | 0.117 | 0.105 |

| Nortro- | 0.014 | >12 | | 0.012 | (0.001) | >0.3 | 0.069 | 0.075 | 0.001 |
|---|---|---|---|---|---|---|---|---|---|
| pisetron | | | | | | | | | |
| PHA- | >0.3 | >3 | | 0.036 | (0.006) | >1 | 0.111 | 0.057 | 0.024 |
| 543613 | | | | | | | | | |
| Granisetron | 0.2 | 0.06 | (0.01) | 6.8 | (1.7) | 4.8 | >30 | 0.84 | >30 |

| | | | | | | | |
|---|---|---|---|---|---|---|---|
| Ivermectin | nd | nd | 0.21 | nd | nd | nd | nd |

nd = not determined

For use in organisms that produce ACh, it is important to reduce the endogenous ACh potency at these channels comprised of the α7 nAChR LBD. Mutation G175K could be further combined with other mutations that reduced sensitivity to ACh, such as Y115F and Y210F. For α7^{Y115F,G175K}-GlyR, high potency for agonists based on tropane or quinuclidine core structures were found for tropisetron, granisetron, nortropisetron, PNU-282987, and PHA-543613, and greatly reduced potency for varenicline and cytisine (Table 5). For α7^{G175K,Y210F}-GlyR, potency for most agonists was considerably reduced, however potency enhancement for granisetron was observed (Table 5).

To develop channels with reduced ACh responsiveness but high potency for other agonists, α7G175K-GlyR was combined with additional mutations that increase the potency of specific agonists. Combination with W77F reduced ACh potency, and α7^{W77F,G175K}-GlyR showed increased potency over α7-GlyR for granisetron, nortropisetron, and tropisetron but not for PNU282-987, varenicline, cytisine, or PHA-543613 (Table 5). Combination of G175K with Q79G reduced ACh potency, and α7^{Q79G,G175K}-GlyR showed increased potency for nortropisetron, PHA-543613, and tropisetron (Table 5). However, this potency enhancement was not observed for other agonists, such as PNU282-987, or varenicline. α7^{G175K,Q139L}-GlyR reduced ACh potency and increased potency for nortropisetron and tropisetron (Table 5).

Further reductions in ACh potency were achieved while maintaining high potency for with synthetic agonists, including those based on tropane and quinuclidine core structures, by incorporating mutations at W77F, Q79G, L141F, Y115F, G175K, and Y210F in various combinations. α7^{Q79G,Y115F,G175K}-GlyR reduced ACh responsiveness while maintaining potent responses to tropisetron (Table 5). These mutations also enhanced responsiveness to other tropane and quinuclidine core structures relative to α7^{Y115F,G175K}-GlyR as well as relative to α7-5HT3 (representative of endogenous α7 nAChR activity), especially quinuclidine thioureas 702 and 703 as well as tropane ester 723, 725, 726, 736, 737, 738, and 745 (Table 6). α7^{Q79G,Y115F,G175K}-GlyR also showed high sensitivity to ivermectin (Table 5). α7^{W77F,Q79G,G175K}-GlyR reduced ACh responsiveness while maintaining high potency responses to tropisetron, and nortropisetron (Table 5). α7^{W77F,Q79G,G175K}-GlyR also showed enhanced potency for additional tropane-based core structures, such as compounds 723 and 725, as well as the clinically used drugs mequitazine and promazine (Table 6). α7^{W77F,G175K,Y210F}-GlyR reduced ACh responsiveness but markedly improved potency to granisetron (Table 5). α7^{L141F,Y115F,G175K}-GlyR reduced ACh responsiveness while conferring sensitivity to granisetron (Table 5). α7^{Q79G,Q139L,G175K}-GlyR reduced ACh responsiveness but showed potent responses to nortropisetron (Table 5).

TABLE 6
Potency enhancement of tropane, quniuclidine agonists, 9-azabicyclo[3.3.1]nonane agonists, diazabicyclo[3.2.2]nonane agonists, and promazine by G175K and P216I α7GlyR chimeric
channels. Indole and indazole aromatic (A) substituents attached at 3-position. Units: μM.

| | | | | | | | | | | | | | | | | | | α7GlyR | |
|---|---|---|---|---|---|---|---|---|---|---|---|---|---|---|---|---|---|---|---|
| | | | | | | | | | | | | | | | | | | Q79G | |
| | | | | | | | | | | | | | | | | | α7Gly | G175K | α7GlyR |
| | | | | | | | | C-X | | | | | | | α7GlyR | α7GlyR | Q79G | Y115F | W77F |
| | | | | | | | | config- | | Aromatic substitution | Com- | α7- | α7- | α7GlyR | Q79G | Y115F | G175K | R27D | Q79G |
| Agonist class | X₁ | X₂ | X₃ | Y | C₁ n | C₂ n | C₃ n | uration | R | (A) | pound | 5HT3 | GlyR | G175K | G175K | G175K | Y115F | E41R | G175K |
| Quinuclidine | N | CH₂ | NH | S | 0 | 1 | 0 | R | H | 3,5-dichloro-aniline | 677 | 10.6 | 4.4 | 0.66 | 0.86 | 3.7 | 0.98 | 0.58 | nd |
| | | | | | | | | | | | | | | (0.06) | (0.004) | (0.7) | (0.09) | (0.14) | |
| Quinuclidine | N | CH₂ | NH | S | 0 | 1 | 0 | R | H | 3,4-dichloro-aniline | 682 | >100 | 0.2 | 0.12 | 0.013 | 0.40 | 0.13 | 0.06 | nd |
| | | | | | | | | | | | | | | (0.1) | (0.001) | (0.01) | (0.01) | (0.012) | |
| Quinuclidine | N | CH₂ | NH | S | 0 | 1 | 0 | R | H | 4- | 684 | >100 | 1.6 | 0.23 | 0.078 | 3.0 | 0.79 | 0.4 | nd |
| | | | | | | | | | | (trifluoromethoxy)aniline | | | | (0.02) | (0.022) | (0.3) | (0.04) | (0.03) | |
| Quinuclidine | N | CH₂ | NH | S | 0 | 1 | 0 | R | H | 4-fluoroaniline | 699 | 2.8 | 3.6 | 0.26 | 0.039 | 2.9 | 0.52 | 0.33 | nd |
| | | | | | | | | | | | | | | (0.11) | (0.009) | | (0.09) | (0.1) | |
| Quinuclidine | N | CH₂ | NH | S | 0 | 1 | 0 | R | H | 3-chloro-aniline | 700 | 1.8 | 1.9 | 0.081 | 0.012 | 1.5 | 0.21 | 0.11 | nd |
| | | | | | | | | | | | | | | (0.009) | (0.0002) | | (0.04) | (0.02) | |
| Quinuclidine | N | CH₂ | NH | S | 0 | 1 | 0 | R | H | 3-chloro-2-fluoroaniline | 701 | >100 | nd | 0.47 | 0.086 | 5.46 | 1.0 | 0.58 | nd |
| | | | | | | | | | | | | | | (0.17) | (0.014) | | (0.2) | (0.03) | |
| Quinuclidine | N | CH₂ | NH | S | 0 | 1 | 0 | R | H | 3-chloro-4-fluoroaniline | 702 | >100 | 0.9 | 0.12 | 0.018 | 1.6 | 0.17 | 0.12 | nd |
| | | | | | | | | | | | | | | (0.004) | (0.003) | | (0.03) | (0.02) | |
| Quinuclidine | N | CH₂ | NH | S | 0 | 1 | 0 | R | H | 5-chloro-2-fluoroaniline | 703 | >100 | nd | 0.52 | 0.03 | 12.7 | 1.2 | 1.1 | nd |
| | | | | | | | | | | | | | | (0.08) | (0.01) | | (0.06) | (0.5) | |
| Quinuclidine | N | CH₂ | NH | S | 0 | 1 | 0 | R | H | 3-chloro-4-methylaniline | 704 | 0.7 | nd | 0.062 | 0.018 | 0.76 | 0.24 | 0.18 | nd |
| | | | | | | | | | | | | | | (0.008) | (0.002) | (0.01) | (0.02) | (0.06) | |
| Quinuclidine | N | CH₂ | NH | S | 0 | 1 | 0 | R | H | 5-chloro-2-methylaniline | 705 | >100 | nd | 9.6 | 0.67 | >10 | 4.8 | 4.5 | nd |
| | | | | | | | | | | | | | | | (0.14) | | (1.4) | (2.7) | |
| Quinuclidine | N | CH₂ | NH | S | 0 | 1 | 0 | S | H | 4- | 713 | >100 | nd | 2.1 | 0.54 | >10 | 23.9 | >10 | nd |
| | | | | | | | | | | (trifluoromethoxy)aniline | | | | (0.2) | (0.06) | | | | |
| Tropane | C | NMe | NH | S | 1 | 0 | 0 | Endo | H | 1-methyl-1H-indole | 622 | >100 | nd | 0.87 | 1.3 | 2.5 | 0.93 | 1.0 | 1.7 |
| | | | | | | | | | | | | | | | (0.2) | (0.4) | (0.02) | (0.2) | |
| Tropane | C | NMe | O | O | 1 | 0 | 0 | Endo | H | 4-methoxy-1H-indole | 721 | 0.5 | nd | 0.027 | 0.015 | 0.080 | 0.020 | 0.016 | 0.04 |
| | | | | | | | | | | | | | | (0) | (0.003) | (0.002) | (0.001) | (0.001) | |
| Tropane | C | NMe | O | O | 1 | 0 | 0 | Endo | H | 6-methoxy-1H-indole | 722 | 0.5 | nd | 0.02 | 0.015 | 0.052 | 0.028 | 0.016 | 0.03 |
| | | | | | | | | | | | | | | (0.001) | (0) | (0.008) | (0.008) | (0.001) | |
| Tropane | C | NMe | O | O | 1 | 0 | 0 | Endo | H | 7-methoxy-1H-indole | 723 | 12.8 | 4 | 0.31 | 0.02 | 0.71 | 0.07 | 0.024 | 0.02 |
| | | | | | | | | | | | | | | (0.02) | (0) | (0.46) | (0.01) | (0.003) | |
| Tropane | C | NMe | O | O | 1 | 0 | 0 | Endo | H | 4-methyl-1H-indole | 724 | 1.2 | nd | 0.036 | 0.012 | 0.091 | 0.02 | 0.012 | 0.06 |
| | | | | | | | | | | | | | | (0.003) | (0.002) | (0.013) | (0.006) | (0.002) | |
| Tropane | C | NMe | O | O | 1 | 0 | 0 | Endo | H | 7-methyl-1H-indole | 725 | 12.2 | 8.1 | | 0.022 | 0.069 | 0.042 | 0.022 | 0.024 |
| | | | | | | | | | | | | | | | (0.02) | (0.33) | (0.005) | (0.0001) | |
| Tropane | C | NMe | O | O | 1 | 0 | 0 | Endo | H | 4-chloro-1H-indole | 726 | 4.2 | nd | 0.58 | 0.016 | 0.51 | 0.044 | 0.018 | 0.03 |
| | | | | | | | | | | | | | | (0.24) | (0.001) | (0.37) | (0.006) | (0) | |
| Tropane | C | NMe | O | O | 1 | 0 | 0 | Endo | H | 5-methoxy-1H-indole | 736 | 0.83 | nd | 0.2 | 0.044 | 0.57 | 0.078 | 0.078 | 0.06 |
| | | | | | | | | | | | | | | (0.01) | (0.002) | (0.21) | (0.018) | (0.024) | |
| Tropane | C | NMe | O | O | 1 | 0 | 0 | Endo | H | 5-chloro-1H-indole | 737 | 1 | 0.9 | 0.082 | 0.013 | 0.16 | 0.033 | 0.016 | 0.101 |
| | | | | | | | | | | | | | | (0.004) | (0.001) | (0.03) | (0.004) | (0.001) | |
| Tropane | C | NMe | O | O | 1 | 0 | 0 | Endo | H | 6-chloro-1H-indole | 738 | 0.4 | nd | 0.015 | 0.016 | 0.04 | 0.025 | 0.012 | 0.033 |
| | | | | | | | | | | | | | | (0) | (0.002) | (0.014) | (0.002) | (0.001) | |
| Tropane | C | NMe | O | O | 1 | 0 | 0 | Endo | H | 1H-indazole | 745 | 1.2 | 1.3 | 0.069 | 0.026 | 0.26 | 0.089 | 0.043 | 0.05 |
| | | | | | | | | | | | | | | | (0.002) | (0.03) | (0.024) | (0.014) | |
| 9- | CH | NMe | NH | O | 1 | 0 | 1 | Endo | H | 1H-indole | 749 | 6.6 | nd | nd | nd | nd | 1.3 | nd | 1.9 |
| azabicyclo | | | | | | | | | | | | | | | | | | | |
| [3.3.1]nonane | | | | | | | | | | | | | | | | | | | |
| 9- | CH | NMe | NH | O | 1 | 0 | 1 | Endo | H | 1H-indazole | 751 | 1.8 | 3.4 | nd | nd | nd | 3.2 | nd | 0.7 |
| azabicyclo | | | | | | | | | | | | | | | | | | | |
| [3.3.1]nonane | | | | | | | | | | | | | | | | | | | |
| 9- | CH | NMe | NH | O | 1 | 0 | 1 | Endo | H | 7-methoxy-1H-indazole | 760 | >100 | 9.8 | nd | nd | nd | 3 | nd | 1.3 |
| azabicyclo | | | | | | | | | | | | | | | | | | | |
| [3.3.1]nonane | | | | | | | | | | | | | | | | | | | |
| 9- | CH | NH | O | O | 1 | 0 | 1 | Endo | H | 1H-indole | 763 | 1.9 | 0.17 | nd | nd | nd | 0.3 | nd | 0.2 |
| azabicyclo | | | | | | | | | | | | | | | | | | | |
| [3.3.1]nonane | | | | | | | | | | | | | | | | | | | |
| 1,4- | | | | | | | | | F | dibenzo[b,d]thiophene | 773 | 0.135 | 0.001 | nd | nd | 0.0003 | 0.00042 | nd | 0.0014 |
| diazabicyclo | | | | | | | | | | 5,5-dioxide | | | | | | | | | |
| [3.2.2]nonane | | | | | | | | | | | | | | | | | | | |
| 1,4- | | | | | | | | | NO₂ | dibenzo[b,d]thiophene | 774 | 0.03 | 0.006 | nd | nd | 0.00078 | 0.03 | nd | 0.03 |
| diazabicyclo | | | | | | | | | | 5,5-dioxide | | | | | | | | | |
| [3.2.2]nonane | | | | | | | | | | | | | | | | | | | |
| Quinuclidine | N | CH₂ | CH₂ | | 0 | 1 | 0 | R | H | 10H-phenothiazine | Mequi- | >30 | nd | nd | nd | nd | >10 | nd | 0.15 |
| | | | | | | | | | | | tazine | | | | | | | | |
| N,N- | | | | | | | | | | 10H-phenothiazine | Pro- | >100 | nd | nd | nd | nd | >100 | nd | 1.6 |
| dimethylpropyl | | | | | | | | | | | mazine | | | | | | | | |
| amine | | | | | | | | | | | | | | | | | | | |

nd = not determined;
parentheses: SEM

α7^{G175K}-GlyR and α7^{P216I}-GlyR along with mutations at Q79G, Y115F, and G175K were also compatible with non-association mutations R27D,E41R as well as the GlyR IPD mutation A298G, which further enhanced ligand potency for granisetron, epibatidine, varenicline, cytisine, PNU-282987, tropisetron, nortropisetron, and PHA-543613 (Table 7). Combination with non-association mutations to form α7^{R27D,E41R,Q79G,Y115F,G175K} further improved the potency for 702, 723, 725, and 726, with low ACh responsiveness (Table 6).

TABLE 7
Agonist potency enhancement by G175K and A298G mutations at α7GlyR chimeric
channels as well as W298A at α7GABAc (also referred to as GABA_{A}-ρ) channels. Units:
μM.

| | | | | | α7GlyR | | α7GlyR | |
|---|---|---|---|---|---|---|---|---|
| | | | α7GlyR | | ^{Q79G} | | ^{Q79G} | α7GlyR |
| | α7GlyR | α7GlyR | ^{Q79G} | α7GlyR | ^{A298G} | a7GABAc | ^{G175K} | ^{R27D} |
| | ^{Q79G} | ^{Q79G} | ^{A298G} | ^{Q79G} | ^{Y115F} | ^{Q79G} | ^{Y115F} | ^{E41R} |
| | ^{W77F} | ^{G175K} | ^{G175K} | ^{A298G} | ^{K395} | ^{L141F} | ^{R27D,} | ^{Q79G} |
| Compound | ^{A298G} | ^{A298G} | ^{Y115F} | ^{P216I} | ^{K396A} | ^{W298A} | ^{E41R} | ^{Y115F} |
| Acetylcholine | 45 | 0.66 | 31 | 5 | 90 | 52 | 52 (7.7) | >500 |
| Nicotine | 3.8 | 0.11 | 3.3 | 1.6 | 16.5 | 16.2 | 4.8 (0.4) | >39.8 |
| Epibatidine | 0.37 | 0.0023 | 0.011 | 0.05 | 0.15 | 0.42 | 0.059 (0.03) | 0.267 |
| Varenicline | 3.66 | 0.022 | 2.37 | 0.18 | >30 | 6.27 | 4.9 (0.3) | >30 |
| Cytisine | 14.1 | 0.134 | 4.6 | 5.5 | >30 | 13.3 | 4.8 (0.4) | >30 |
| PNU-282987 | 1.63 | 0.00036 | 0.009 | 0.25 | 0.11 | 0.12 | 0.05 (0.03) | 0.34 |
| Tropisetron | 0.018 | 0.0006 | 0.0028 | 0.009 | 0.021 | 0.111 | 0.013 (0.005) | >0.096 |
| Nortropisetron | 0.0024 | 0.00013 | 0.0084 | 0.0012 | 0.0063 | 0.009 | 0.003 (0.001) | 0.102 |
| PHA-543613 | 0.0066 | 0.00018 | 0.0039 | 0.003 | 0.0408 | 0.039 | 0.0054 | 0.156 |
| Granisetron | 1.2 | nd | nd | nd | >30 | >100 | 2.4 (0.3) | >30 |

nd = not determined;
parentheses: SEM

Additional amino acid substitutions at Gly¹⁷⁵ of the α7 nAChR LBD in α7Y115F-GlyR chimeric channels are also enhanced agonist potency. Potency for tropisetron at α7Y115F-GlyR chimeric channels was enhanced with additional mutations, which include G175A (7.1-fold), G175F (2-fold), G175H (2.3-fold), G175K (5.6-fold), G175M (2.6-fold), G175R (5.8-fold), G175S (9.3-fold), G175V (16.7-fold).

TABLE 8
Agonist potency enhancement by G175 mutations at α7GlyR Y115F chimeric
channels. Units: μM.

| | | α7GlyR | α7GlyR | α7GlyR | α7GlyR | α7GlyR | α7GlyR | α7GlyR | α7GlyR |
|---|---|---|---|---|---|---|---|---|---|
| | | Y115F | Y115F | Y115F | Y115F | Y115F | Y115F | Y115F | Y115F |
| Compound | a7GlyR | G175K | G175A | G175F | G175H | G175M | G175R | G175S | G175V |
| Acetylcholine | 6.4 (1.2) | 52 (6.6) | 24 | 67 | 79 | 71 | 29.5 | 31.5 | 15 |
| Varenicline | 0.62 | 5.0 (1.7) | 5.9 | 13.6 | 12.7 | 14.1 | 7.6 | 9.7 | 4.6 |
| | (0.2) | | | | | | | | |
| Tropisetron | 0.15 | 0.027 | 0.021 | 0.074 | 0.064 | 0.057 | 0.024 | 0.016 | 0.009 |
| | (0.045) | (0.004) | | | | | | | |
| PHA-543613 | 0.03 | 0.02 | 0.027 | 0.173 | 0.12 | 0.25 | 0.11 | 0.12 | 0.037 |
| | (0.01) | (0.007) | | | | | | | |

nd = not determined;
parentheses: SEM

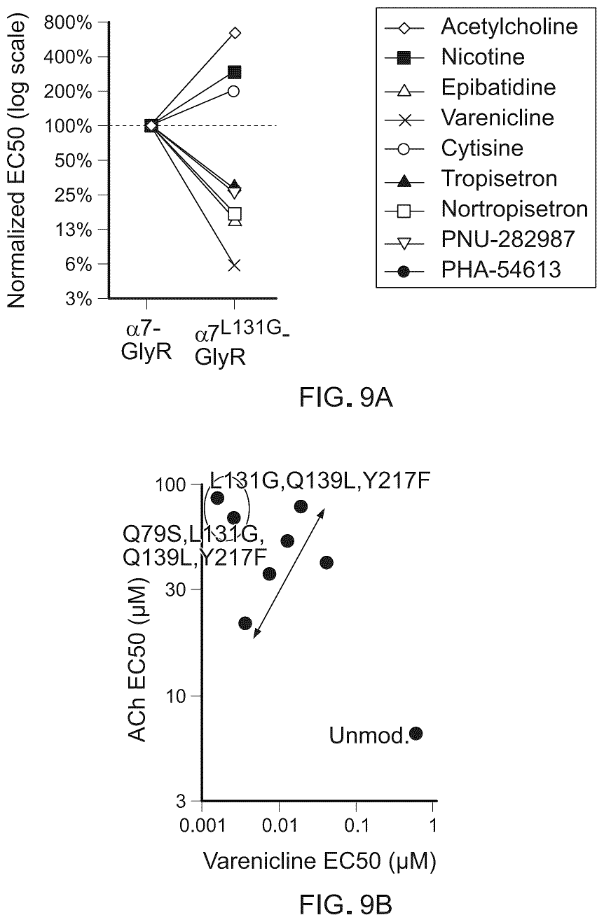

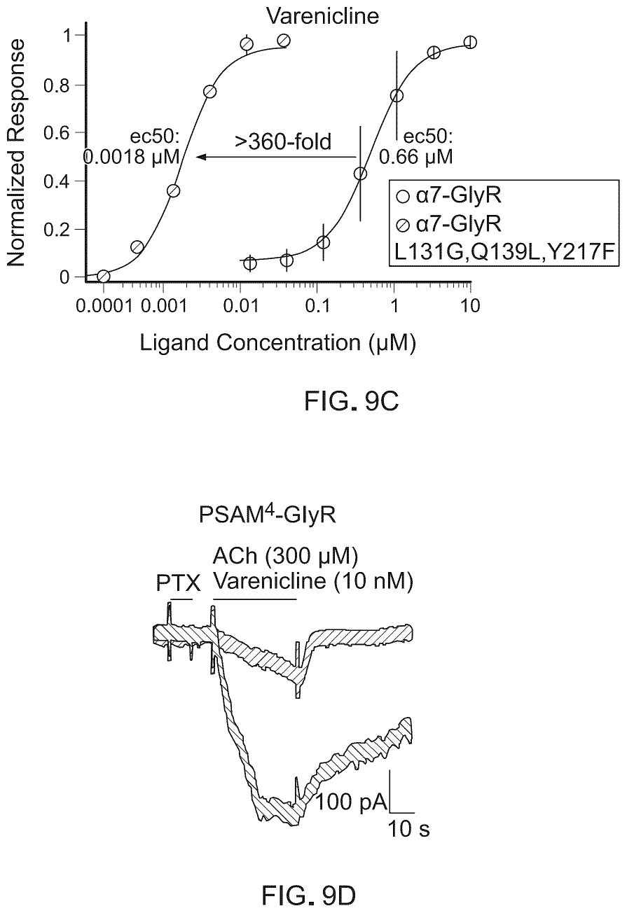

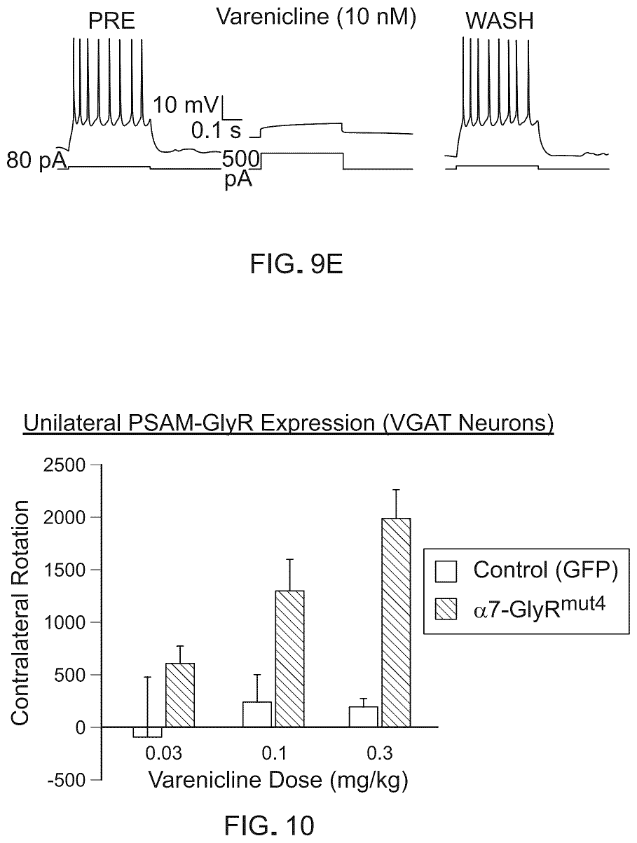

Mutations for Leu¹³¹ to smaller amino acids were found to reduce the potency of canonical agonists ACh and nicotine, while markedly increasing potency of varenicline, tropisetron and several other agonists. α7^{L131A}-GlyR and α7^{L131G}-GlyR had reduced ACh responsiveness (6-fold) and enhanced potency for varenicline (8-fold and 17-fold, respectively) and tropisetron (2.5-fold and 3.6-fold, respectively) (Table 9). α7L131G-5HT3 HC had reduced ACh responsiveness (5-fold) and enhanced potency for varenicline (16-fold) and tropisetron (2.3-fold) (FIG. 9A and Table 9). α7^{L131G,Q139L}-GlyR and α7^{L131G,Y217F}-GlyR showed similar potency enhancement over α7-GlyR for varenicline (21-fold) but also reduced ACh sensitivity (−11-fold and −13-fold, respectively). α7^{Q79S,L131G}-GlyR further improved potency over α7-GlyR for varenicline (89-fold) and tropisetron (15-fold). α7^{L131G,Q139L,Y217F}-GlyR showed the greatest improvement in potency over α7-GlyR for varenicline (387-fold) and also showed reduced ACh potency (13-fold) (FIG. 9B and Table 9). α7^{L131G,Q139L,Y217F}-GlyR also showed extremely high potency for compound 770 (0.001 μM), compound 773 (0.00034 μM), and compound 774 (0.00013 μM) (FIG. 11). α7^{Q79S,L131G,Q139L}-GlyR also improved potency over α7-GlyR for varenicline (31-fold) and tropisetron (3-fold) but reduced ACh potency (9-fold) (FIG. 9B and Table 9). α7^{L131M}-GlyR, α7^{L131Q}-GlyR, and α7L131V-GlyR reduced ACh potency but enhanced potency to tropisetron, nortropisetron, PHA-543613, and granisetron (Table 9). α7^{L131F}-GlyR was found to substantially reduced ACh potency but did not improve potency for other agonists (Table 8). α7^{L131G}-GABAc substantially reduced ACh potency but did not improve potency for other agonists (Table 9). α7^{L131G,Q139L,Y217F}-5HT3 HC (Table 9) improved varenicline potency by 131-fold over α7-5HT3 (Table 1). α7^{L131G,Q139I,Y217F}-5HT3 HC also showed high potency for compound 770 (0.007 UM), compound 773 (0.002 μM), and compound 774 (0.004 μM) (Table 8).

TABLE 9
Agonist potency enhancement by chimeric channels with L131 mutations. Units: μM.

| | | | | | | α7GlyR | | | α7GlyR | |
|---|---|---|---|---|---|---|---|---|---|---|
| | | | | α7GlyR | α7GlyR | L131G | α7GlyR | α7GlyR | Q79S | α7GlyR |
| | | α7GlyR | α7GlyR | L131G | L131G | Q139L | Q79G | Q79S | L131G | L131G |
| Compound | a7GlyR | L131A | L131G | Q139L | Y217F | Y217F | L131G | L131G | Q139L | D219A |
| Acetyl- | 6.4 | 42 | 41 | 68 | 85 | 83 | >500 | 21 | 58 | 210 |
| choline | (1.2) | (21) | (11) | | | (20) | | (3.5) | | |
| Nicotine | 5.0 | 8.0 | 15 | 26 | 28 | 55 | >100 | 8.2 | 25 | 36 |
| | (1.8) | (3.2) | (3.5) | | | (18) | | (0.8) | | |
| Epibatidine | 0.062 | 0.027 | 0.009 | 0.012 | 0.015 | 0.021 | nd | 0.007 | 0.012 | 0.16 |
| | (0.021) | | (0.004) | | | (0.002) | | (0.001) | | |
| Varenicline | 0.62 | 0.082 | 0.037 | 0.03 | 0.03 | 0.0016 | >10 | 0.007 | 0.02 | 0.78 |
| | (0.2) | (0.068) | (0.026) | | | (0.001) | | (0.001) | | |
| Cytisine | 6.4 | 20.6 | 13.1 | 12 | 30 | nd | >30 | 8.1 | 10 | >30 |
| | (2.0) | (9.4) | (0.66) | | | | | (0.3) | | |
| PNU- | 0.13 | 0.055 | 0.034 | 0.063 | 0.054 | 0.16 | 0.096 | 0.006 | 0.018 | 0.41 |
| 282987 | (0.038) | (0.025) | (0.008) | | | (0.03) | | (0.002) | | |
| Tropisetron | 0.15 | 0.06 | 0.042 | 0.13 | 0.087 | 0.31 | 0.09 | 0.01 | 0.045 | 0.36 |
| | (0.045) | (0.021) | (0.01) | | | (0.05) | | (0.003) | | |
| Nortro- | 0.022 | 0.006 | 0.004 | 0.024 | 0.018 | 0.047 | 0.012 | 0.004 | 0.006 | 0.07 |
| pisetron | (0.007) | (0.003) | (0.001) | | | (0.006) | | (0.002) | | |
| PHA- | 0.03 | 0.012 | 0.008 | 0.021 | 0.016 | 0.045 | 0.066 | 0.002 | 0.009 | 0.038 |
| 543613 | (0.01) | (0.006) | (0.002) | | | (0.008) | | (0.0005) | | |
| Granisetron | >100 | 17.2 | 6.7 | 4 | 4 | nd | nd | 4.2 | nd | >30 |
| | | (12.8) | (1.6) | | | | | (0.8) | | |
| 765 | >100 | nd | nd | nd | nd | 0.031 | 0.027 | 0.024 | nd | nd |
| | | | | | | (0.02) | | | | |
| 770 | nd | nd | nd | nd | nd | 0.001 | nd | nd | nd | nd |
| | | | | | | (0.0003) | | | | |
| 773 | 0.001 | nd | 0.00013 | 0.00004 | nd | 0.00034 | 0.00004 | nd | nd | nd |
| 774 | 0.006 | nd | 0.00004 | 0.00004 | nd | 0.00018 | 0.00004 | nd | nd | nd |
| | | α7GlyR | | | | | | | α75HT3 | |
| | | Q79S | | | | | | | L131G | |
| | | L131G | | α7GlyR | | | | α75HT3 | Q139L | α7- |
| | α7GlyR | Q139L | α7GlyR | Y115F | α7GlyR | α7GlyR | α7GlyR | L131G | Y217F | GABA_{C} |
| Compound | L131F | Y217F | L131M | L131M | L131N | L131Q | L131V | HC | HC | L131G |
| Acetyl- | 92 | 67 | 29 | >500 | 5 | 58 | 16 | 35 | 39 | >500 |
| choline | (32) | (3) | | | (0.5) | | (5) | | | |
| Nicotine | 20 | 41 | 15 | nd | nd | 13 | 3.9 | 15 | 20 | >500 |
| | (6.3) | (8) | | | | | (0.7) | | | |
| Epibatidine | 0.24 | 0.022 | 0.042 | nd | nd | 0.027 | 0.21 | 0.009 | nd | |
| | (0.05) | (0.004) | | | | | (0.04) | | | |
| Varenicline | 2.6 | 0.003 | 0.53 | >100 | 0.069 | 0.72 | 0.33 | 0.04 | 0.007 | 0.3 |
| | (1.1) | (0.001) | | | (0.027) | | (0.21) | | | |
| Cytisine | 10.5 | nd | 7 | nd | nd | >30 | 4.3 | 11 | nd | >500 |
| | (1.8) | | | | | | (0.7) | | | |
| PNU- | 0.20 | 0.05 | 0.021 | nd | nd | 0.048 | 0.064 | 0.033 | 0.015 | 0.12 |
| 282987 | (0.04) | (0.01) | | | | | (0.018) | | | |
| Tropisetron | 0.39 | 0.084 | 0.024 | 0.035 | 0.025 | 0.048 | 0.062 | 0.066 | 0.04 | 0.18 |
| | (0.2) | (0.009) | | | (0.005) | | (0.013) | | | |
| Nortro- | 0.027 | 0.014 | 0.006 | nd | nd | 0.009 | 0.003 | 0.009 | nd | 0.021 |
| pisetron | (0.008) | (0.002) | | | | | (0.001) | | | |
| PHA- | 0.04 | 0.015 | 0.009 | 0.028 | 0.02 | 0.015 | 0.011 | 0.012 | 0.009 | 0.027 |
| 543613 | (0.007) | (0.001) | | | | | (0.002) | | | |
| Granisetron | >100 | nd | 4 | nd | nd | 4 | 5.4 | 4 | nd | >500 |
| | | | | | | | (1.3) | | | |
| 765 | nd | 0.034 | nd | nd | >10 | nd | nd | nd | 0.11 | nd |
| | | (0.013) | | | | | | | | |
| 770 | 0.034 | 0.001 | 0.03 | nd | >10 | >0.3 | nd | nd | 0.007 | nd |
| | | (0.0001) | | | | | | | | |
| 773 | 0.0005 | nd | 0.00005 | nd | 0.0004 | 0.006 | nd | nd | 0.002 | nd |
| 774 | 0.0013 | nd | 0.001 | nd | 0.0006 | 0.002 | nd | nd | 0.004 | nd |

nd: not determined;
parentheses: SEM

### Example 7: Chimeric LGIC's in Neurons

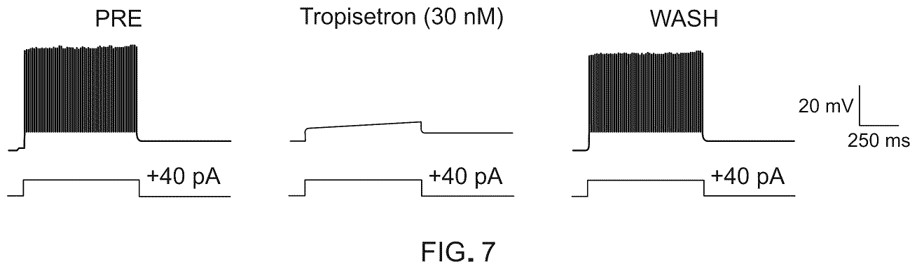

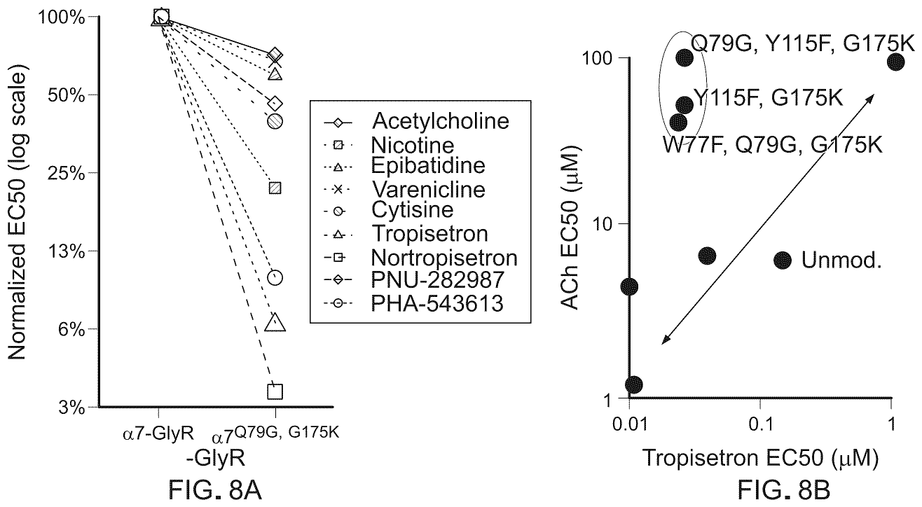

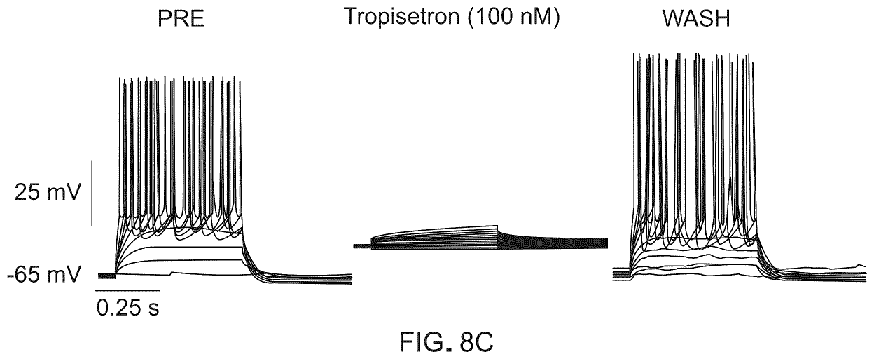

AAVs or DNA plasmids containing nucleic acids encoding a α7979G-GlyR^{A298G} or α7Q79G, Y115F,G175K-GlyR chimeric LGICs were transduced into mouse cortical neurons. A low concentration of tropisetron (30 nM or 100 nM) was administered to mouse cortical neurons. Neuron activity was silenced by application of low concentration of agonist (FIG. 7 and FIG. 8C).

DNA plasmids containing nucleic acids encoding a α7L131G,Q139L,Y217F-GlyR chimeric LGICs were transfected into mouse cortical neurons. Low concentration of varenicline (10 nM) was administered to mouse cortical neurons. Neuron activity was silenced by application of low concentration of agonist (FIG. 9C).

These results show that modified LGIC activity can be controlled in neurons using low concentration of the LGIC ligands tropisetron and varenicline.

### Example 8: Varenicline and Varenicline Derivatives and Chimeric LGICs in Therapy

The anti-smoking drug varenicline is a potent partial α4b2 agonist. Varenicline is also a modest α7 nAChR agonist and an agonist at 5HT3. It has excellent brain penetrance. The chimeric channels α7^{L131G,Q139L,Y217F}-GlyR and α7^{L131G,Q139L,Y217F}-5HT3 HC have enhanced potency binding for the ligand varenicline compared to the unmodified chimeric channels, α7-GlyR and α7-5HT3. However, varenicline activates endogenous ion channels, such as α7 nACHR, α4β2 nAChR, and serotonin receptor 3 (5HT3-R) and it is desirable to obtain varenicline derivatives with high potency at the engineered channels but lowered potency at these endogenous targets.

Using the crystal structure of varenicline bound to the acetylcholine binding protein, it was found that the molecule contacts reside V106, which is homologous to L131 in the α7 nAChR sequence. Mutation to L131G improved varenicline potency by 20-fold while reduce Ach potency 5-fold (FIG. 9A).

Additional mutations to L131G were made to further reduce Ach potency and to improve varenicline potency. In most cases the change in potency was related for both molecules but a subset of mutations selectively enhanced varenicline potency by 360-fold and reduced Ach potency by 20-fold (FIGS. 9B and 9C). You can see the response on the right to 10 nM varenicline compared to 300 μM Ach (FIG. 9D). These chimeric channels show a characteristically slow activation that acts as a low pass filter for transient fluctuations in Ach. Also note that despite the high potency of the channel, it does not have any ligand independent activity as shown by brief application of the channel antagonist picrotoxin before these ligands are added. Ligand independent activity would have registered as an outward current in this trace. When chimeric channels were expressed in cortical neurons, 10 nM varenicline induced ultrapotent neuron silencing (FIG. 9E).

In vivo activity was also very potent in a unilateral substantia nigra (SNr) silencing experiment. Circling contralateral to the silenced SNr was seen at doses above 0.1 milligrams per kilograms (mpk). A 10-fold improvement was seen in varenicline potency over anti-nicotine activity (FIG. 10).

Varenicline derivatives were engineered to reduce or eliminate endogenous varenicline activity. The potency of varenicline and each varenicline derivative was determined for a variety of chimeric channels (Table 10).

TABLE 10
Potency (EC50, μM) of varenicline and varenicline derivatives at chimeric
channels, and comparison to agonist potency at 5HT3-R and α4β2 nAChR.

| | | | | α7GlyR | α75HT3 | | |
|---|---|---|---|---|---|---|---|
| | | | | L131G | L131G | | |
| Compound | | α7- | α7- | Q139L | Q139L | | α4β2 |
| structure | Number | 5HT3 | GlyR | Y217F | Y217F | 5HT3-R | nAChR |
| 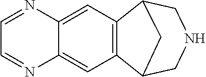 [SMILES: c1cnc2cc3c(cc2n1)C1CNCC3C1] | Varenicline | 0.92 (0.15) | 0.62 (0.2) | 0.0016 (0.001) | 0.004 (0.002) | 1.4 (0.4) | 0.25 (0.06) |
| 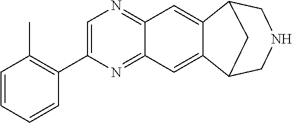 [SMILES: Cc1ccccc1-c1cnc2cc3c(cc2n1)C1CNCC3C1] | 780 | 10.6 | >100 | 0.007 | nd | nd | nd |
| 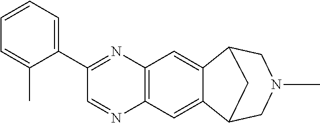 [SMILES: Cc1ccccc1-c1cnc2cc3c(cc2n1)C1CC3CN(C)C1] | 782 | >100 | >100 | 0.64 | 0.36 | >30 | nd |
| 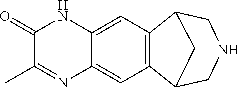 [SMILES: Cc1nc2cc3c(cc2[nH]c1=O)C1CNCC3C1] | 783 | >100 | >100 | 0.008 (0.002) | 0.01 | 0.43 (0.04) | 43 |
| 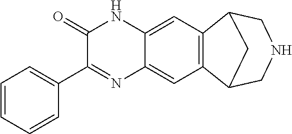 [SMILES: O=c1[nH]c2cc3c(cc2nc1-c1ccccc1)C1CNCC3C1] | 784 | >30 | >30 | 0.43 | 0.88 | 6.2 (0.8) | nd |
| 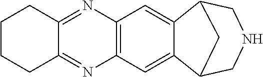 [SMILES: c1c2c(cc3nc4c(nc13)CCCC4)C1CNCC2C1] | 785 | >100 | >100 | 0.25 | 0.12 (0.04) | 10.2 (2.0) | nd |
| 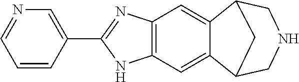 [SMILES: c1cncc(-c2nc3cc4c(cc3[nH]2)C2CNCC4C2)c1] | 786 | >100 | >100 | >10 | >10 | nd | nd |
| 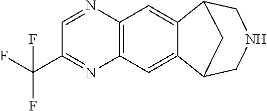 [SMILES: FC(F)(F)c1cnc2cc3c(cc2n1)C1CNCC3C1] | 788 | >100 | >100 | 0.16 (0.02) | 0.13 (0.02) | 41 (1.4) | >30 |
| 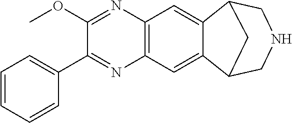 [SMILES: COc1nc2cc3c(cc2nc1-c1ccccc1)C1CNCC3C1] | 789 | >100 | >100 | 0.028 (0.001) | 0.031 (0.002) | 0.51 (0.21) | >30 |
| 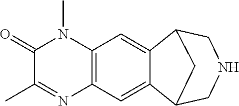 [SMILES: Cc1nc2cc3c(cc2n(C)c1=O)C1CNCC3C1] | 790 | >100 | >100 | 0.048 (0.002) | 0.015 (0.004) | 4.3 (1.1) | >30 |
| 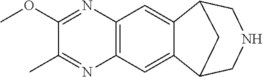 [SMILES: COc1nc2cc3c(cc2nc1C)C1CNCC3C1] | 791 | >30 | >30 | 0.010 (0.003) | 0.003 (0.0005) | 0.17 (0.04) | >30 |
| 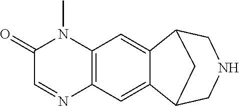 [SMILES: Cn1c(=O)cnc2cc3c(cc21)C1CNCC3C1] | 792 | >30 | >30 | 0.0023 (0.0003) | 0.0016 (0.0002) | 40.5 (0.5) | 0.52 (0.1) |
| 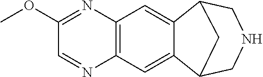 [SMILES: COc1cnc2cc3c(cc2n1)C1CNCC3C1] | 793 | 20 | 19 | 0.0007 (0.0004) | 0.001 (0.0005) | 0.19 (0.055) | 18 (6.1) |
| 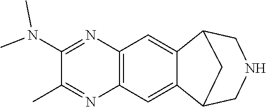 [SMILES: Cc1nc2cc3c(cc2nc1N(C)C)C1CNCC3C1] | 794 | >30 | >30 | 0.071 (0.011) | 0.034 (0.003) | 1.7 (0.37) | >30 |
| 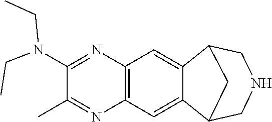 [SMILES: CCN(CC)c1nc2cc3c(cc2nc1C)C1CNCC3C1] | 795 | >30 | >30 | 0.021 (0.004) | 0.016 (0.003) | 1.1 (0.2) | >30 |
| 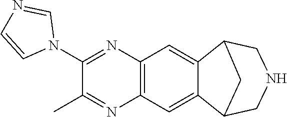 [SMILES: Cc1nc2cc3c(cc2nc1-n1ccnc1)C1CNCC3C1] | 798 | >30 | >30 | 0.020 (0.001) | 0.006 (0.001) | 1.1 (0.24) | 28 (2) |
| 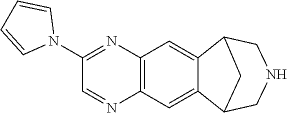 [SMILES: c1ccn(-c2cnc3cc4c(cc3n2)C2CNCC4C2)c1] | 799 | >100 | >100 | 0.74 | 0.42 | 11.7 | 3.5 (1.9) |
| 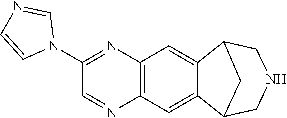 [SMILES: c1cn(-c2cnc3cc4c(cc3n2)C2CNCC4C2)cn1] | 800 | >30 | >30 | 0.11 (0.04) | 0.041 (0.016) | 3.5 (1.9) | 13.9 |
| 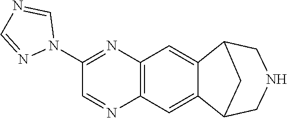 [SMILES: c1ncn(-c2cnc3cc4c(cc3n2)C2CNCC4C2)n1] | 801 | >30 | >30 | 0.074 (0.022) | 0.060 (0.006) | 4.4 (0.9) | 26 (7.4) |
| 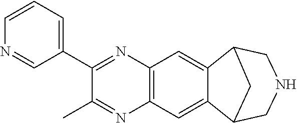 [SMILES: Cc1nc2cc3c(cc2nc1-c1cccnc1)C1CNCC3C1] | 802 | >30 | >30 | 0.003 (0.0006) | 0.001 (0.0004) | 1.5 (0.5) | >30 |
| 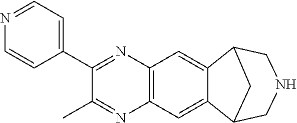 [SMILES: Cc1nc2cc3c(cc2nc1-c1ccncc1)C1CNCC3C1] | 803 | >30 | >30 | 0.007 (0.002) | 0.002 (0.001) | 0.75 (0.22) | 14 (5) |
| 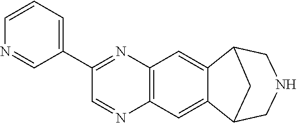 [SMILES: c1cncc(-c2cnc3cc4c(cc3n2)C2CNCC4C2)c1] | 804 | >30 | >30 | 0.020 (0.001) | 0.017 (0.002) | 2.0 (0.48) | 4.8 |
| 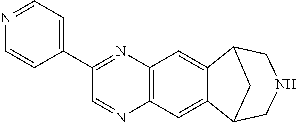 [SMILES: c1cc(-c2cnc3cc4c(cc3n2)C2CNCC4C2)ccn1] | 805 | >30 | >30 | 0.026 (0.003) | 0.030 (0.010) | 1.0 | 4.9 |
| 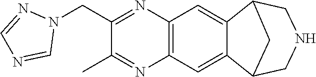 [SMILES: Cc1nc2cc3c(cc2nc1Cn1cncn1)C1CNCC3C1] | 807 | >30 | >30 | 0.007 (0.004) | 0.002 (0.001) | 0.57 | >30 |
| 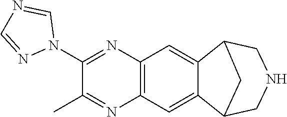 [SMILES: Cc1nc2cc3c(cc2nc1-n1cncn1)C1CNCC3C1] | 808 | >30 | >30 | 0.015 (0.006) | 0.005 (0.0006) | 12.5 (0.5) | >30 |
| 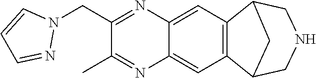 [SMILES: Cc1nc2cc3c(cc2nc1Cn1cccn1)C1CNCC3C1] | 812 | >100 | >100 | 0.03 (0.006) | 0.013 (0.006) | 11 (1) | >100 |
| 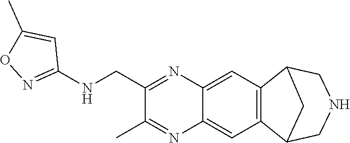 [SMILES: Cc1cc(NCc2nc3cc4c(cc3nc2C)C2CNCC4C2)no1] | 813 | >100 | >100 | 0.017 (0.001) | 0.006 (0.0003) | 3.3 (0.7) | 16.5 (13.5) |

Table 10 shows specific chemical structures of LGIC agonists with substitution patterns compatible with high potency for α7^{L131G,Q139L,Y217F}-GlyR or α7^{L131G,Q139L,Y217F}-5HT3 HC.

All molecules in Table 10 show reduced sensitivity for α7-GlyR and α7-5HT3 (which serve as proxies for α7 nAChR potency). Compound 780, compound 783, compound 789, compound 790, compound 791, compound 792, compound 793, compound 795, compound 798, compound 802, compound 803, compound 804, compound 805, compound 807, compound 808, compound 812, and compound 813 show potency less than 30 nM for either α7^{L131G,Q139L,Y217F}-GlyR or α7^{L131G,Q139L,Y217F}-5HT3 HC. Compound 780, compound 783, compound 791, compound 792, compound 793, compound 798, compound 802, compound 803, compound 807, or compound 808 show potency less than 10 nM for either α7^{L131G,Q139L,Y217F}-GlyR or α7^{L131G,Q139L,Y217F}-5HT3 HC. Compound 792, compound 795, compound 802, compound 808 show potency less than 10 nM for either α7^{L131G,Q139L,Y217F}-GlyR or α7^{L131G,Q139L,Y217F}-5HT3 HC and potency >1 μM for 5HT3-R, and >10 μM for α4β2 nAChR.

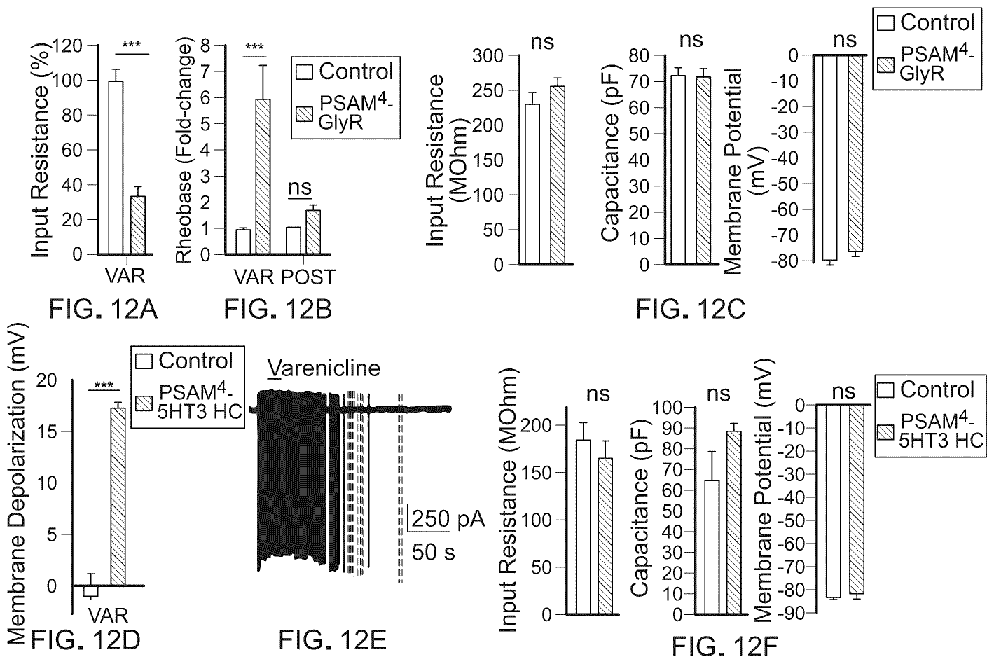

Chemogenetic perturbation of cortical neuron activity was evaluated. Action potential firing was strongly suppressed by varenicline strongly in neurons expressing PSAM⁴-GlyR due to reduced input resistance and elevated rheobase. Cortical layer 2/3 neuron membrane properties were similar in PSAM⁴-GlyR expressing neurons and intermingled untransfected control neurons. Varenicline depolarizes and elicits firing in neurons expressing PSAM⁴-5HT3 HC. Cortical layer 2/3 neuron membrane properties are similar in PSAM⁴-5HT3 HC expressing neurons and intermingled untransfected control neurons. See, e.g., FIG. 12.

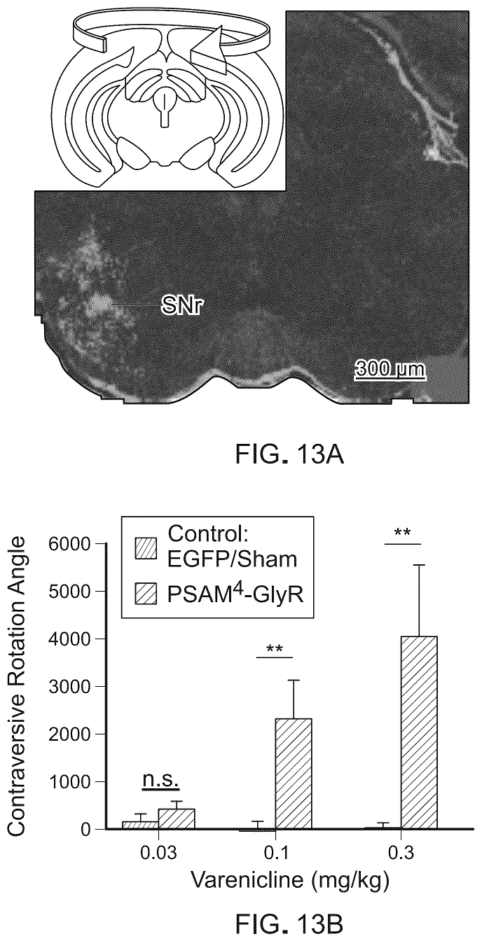

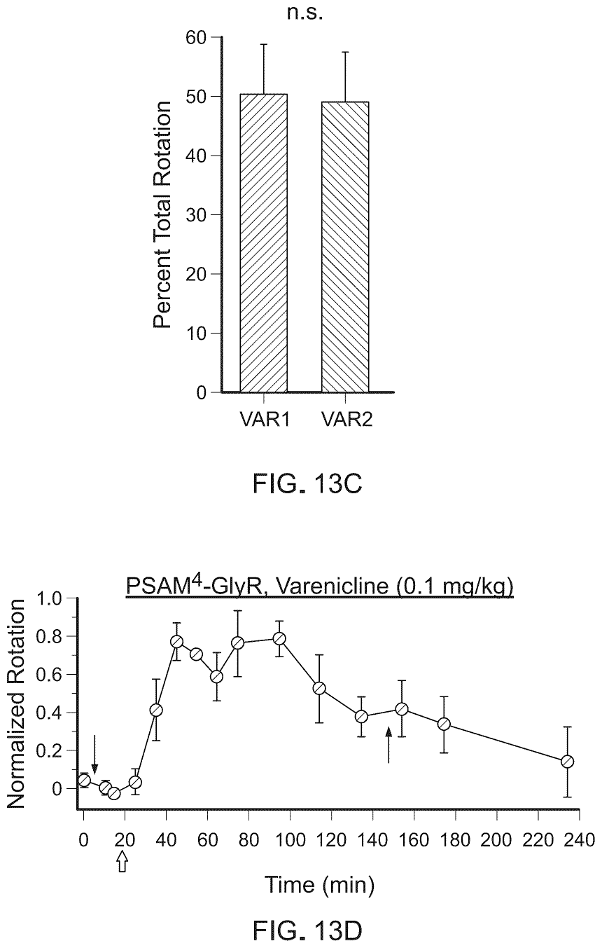

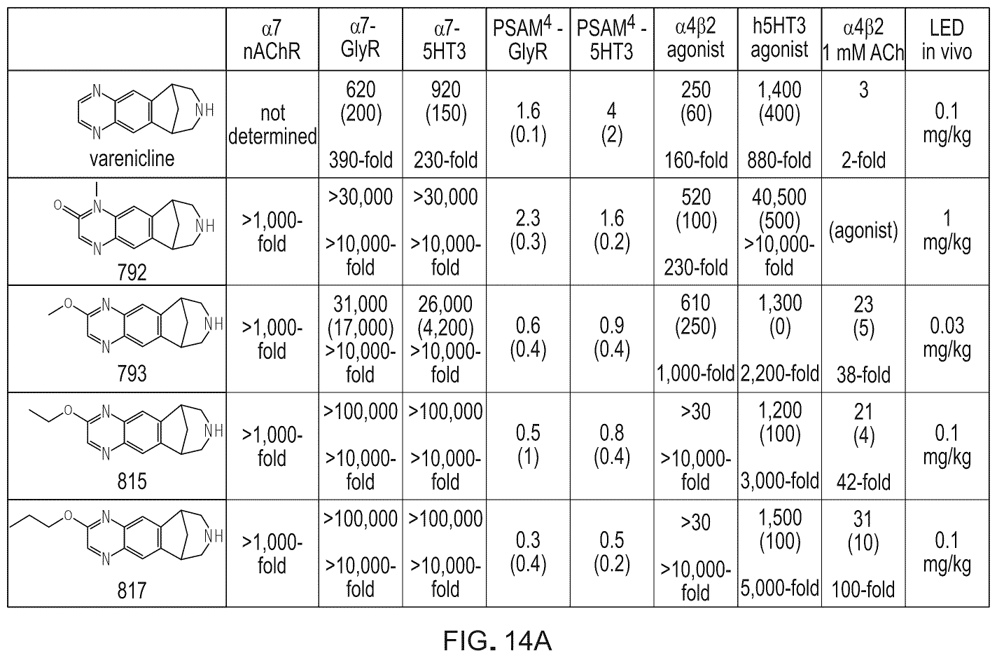

PSAM⁴-GlyR neuron silencing was evaluated in mice. PSAM⁴-GlyR-IRES-EGFP targeted unilaterally to the SNr. Low doses of intraperitoneal varenicline elicit contraversive rotation for mice expressing PSAM⁴-GlyR but not sham operated or EGFP-alone expressing mice. Two doses of varenicline separated by 5 hours give similar proportion of total rotation, indicating no tachyphylaxis of the chemogenetic response. Duration of chemogenetic silencing monitored by the timecourse of rotation response normalized to maximum rotation for each mouse. See, e.g., FIG. 13.

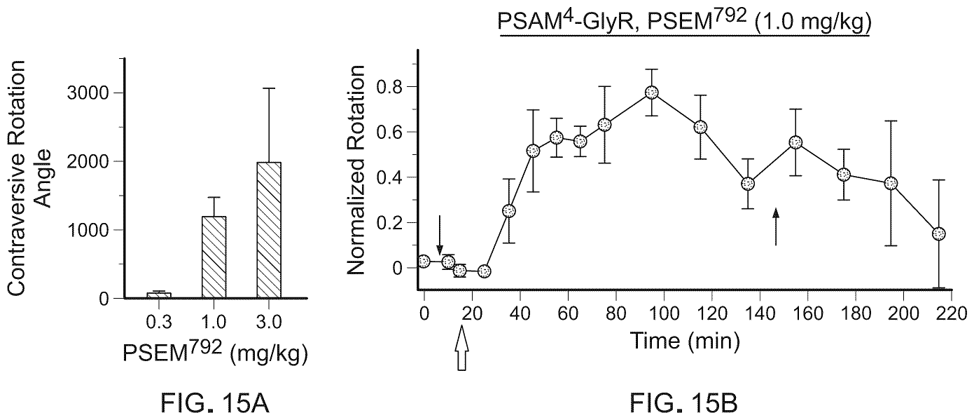

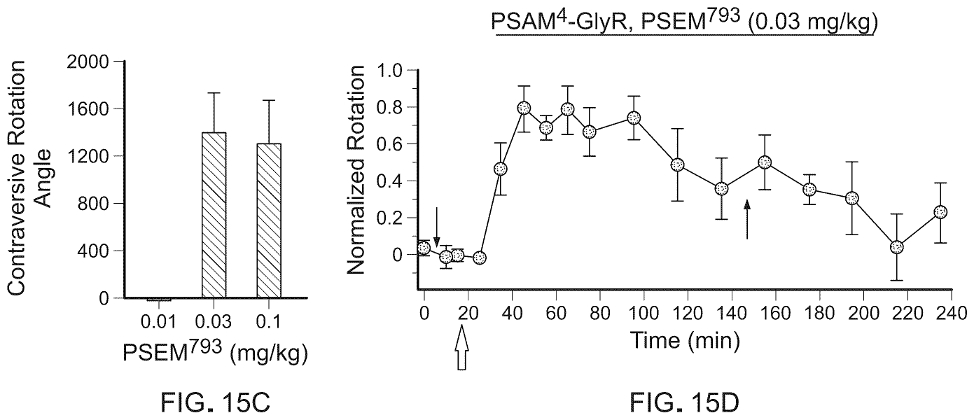

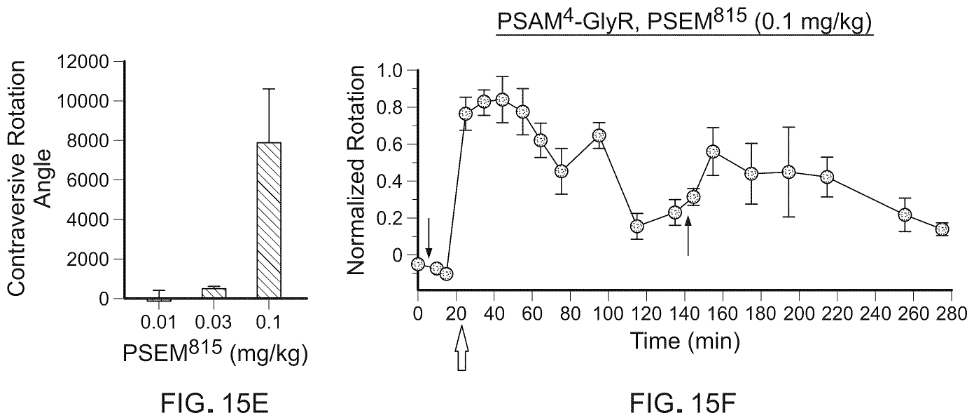

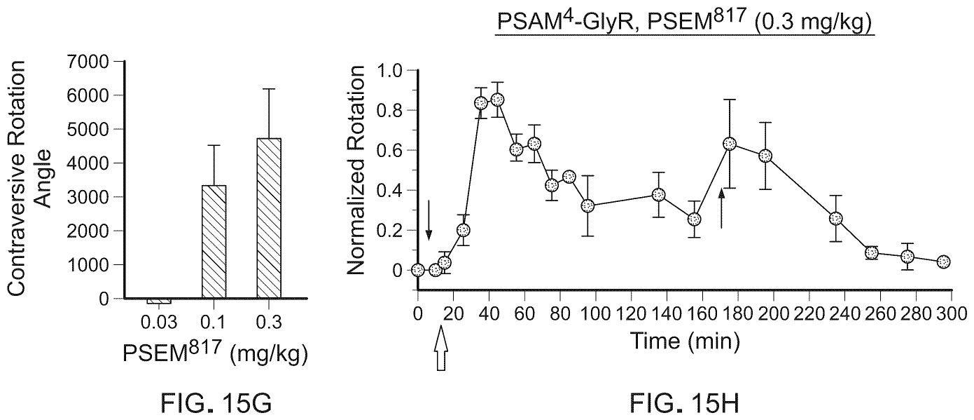

Ultrapotent chemogenetic agonists were evaluated. uPSEM agonist EC50s were compared at PSAM⁴ channels and endogenous varenicline targets, as well as IC50 for α4β2 nAChR with 1 μM ACh. uPSEM⁷⁹² was a 10% partial agonist of α4β2 nAChR and uPSEM⁸¹⁷, and inhibited α482 nAChR. uPSEM⁷⁹², uPSEM⁷⁹³, uPSEM⁸¹⁵, and uPSEM⁸¹⁷ strongly suppressed firing in cortical neurons expressing PSAM⁴-GlyR by reducing the current required to fire an action potential (rheobase). See, e.g., FIG. 14. In vivo uPSEM dose responses were evaluated for mice expressing PSAM⁴-GlyR unilaterally in SNr. See, e.g., FIG. 15.

Varenicline derivatives were engineered to reduce or eliminate endogenous varenicline activity. The potency of varenicline and each varenicline derivative was determined for a variety of chimeric channels (Table 11).

TABLE 11
EC50_{MP} (μM) of varenicline and derivatives at chimeric
channels along with comparison of agonist potency at 5HT3-R and
α4β2 nAChR. In vivo potency determined in SNr silencing rotation assay in mice.

| | | | | | | | α4β2 | In vivo |
|---|---|---|---|---|---|---|---|---|
| Structure | Compound | α7-5HT3 | α7-GlyR | PSAM⁴-GlyR | PSAM⁴-5HT3 | 5HT3-R | nAChR | potency |
| 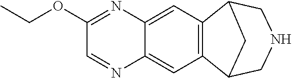 [SMILES: CCOc1cnc2cc3c(cc2n1)C1CNCC3C1] | 815 | >100 | >100 | 0.0005 ± 0.0001 | 0.0008 ± 0.0004 | 1.3 ± 0.0 | >30 | 0.1 mpk |
| 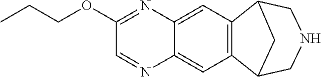 [SMILES: CCCOc1cnc2cc3c(cc2n1)C1CNCC3C1] | 817 | >100 | >100 | 0.0003 ± 0.0001 | 0.0005 ± 0.0002 | 1.5 ± 1.0 | >30 | 0.1 mpk |
| 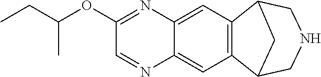 [SMILES: CCC(C)Oc1cnc2cc3c(cc2n1)C1CNCC3C1] | 816 | 33 ± 4 | 52 ± 25 | 0.0005 ± 0.0001 | 0.0009 ± 0.0003 | >100 | 0.56 ± 0.32 | nd |

nd: not determined.
nr: no response.
Values are mean ± SEM.

Ethoxy and propoxy compounds (uPSEMs) 815 and 817 resulted in sub-nanomolar potencies at PSAM⁴-GlyR. uPSEM⁸¹⁷ agonist selectivity was excellent, having 5000-fold to 10,000-fold selectivity for PSAM⁴-GlyR over α7-GlyR, α7-5HT3, and 5HT3-R. uPSEM⁸¹⁵ and uPSEM⁸¹⁷ do not show evident α4β2 nAChR agonism up to 30 μM, and uPSEM 815 has >2000-fold selectivity for PSAM⁴-GlyR over 5HT3-R.

### Example 9: Chimeric LGICs in Therapy

Chemogenetic tools offer an attractive strategy for combined drug and gene therapy. This is because cellular function can be modulated in a consistent manner across different cell types in various indications using the same ion channels and ligands by use of an exogenously delivered ion channel that is selectively engaged by administration of a drug. Identification of ion channels that are gated by well tolerated, clinically used drugs are especially attractive for potentially extending chemogenetics to human therapeutic use.

For the drug tropisetron, we have found that it activates α7^{Q79G}-GlyR^{A298G} with an EC50 of 11 nM, which is similar to the reported IC50 of 10 nM tropisetron for its therapeutic target, the 5HT3 receptor (Combrink et al 2009 Pharmacological reports: PR 61:785-97).

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. (canceled)

2. A synthetic nucleic acid construct encoding a modified ligand gated ion channel (LGIC) subunit comprising:
(i) a human alpha 7 nicotinic acetylcholine receptor (α7-nAChR) ligand binding domain (LBD) comprising an L131G amino acid substitution, a Q139L amino acid substitution, and/or a Y217F amino acid substitution as numbered in SEQ ID NO:2; and
(ii) an ion pore domain (IPD);
wherein an exogenous LGIC ligand selected from the group consisting of compound 0780, compound 0782, compound 0785, compound 0788, compound 0782, compound 0789, compound 0791, compound 0793, compound 0794, compound 0795, compound 0798, compound 0799, compound 0800, compound 0801, compound 0802, compound 0803, compound 0804, compound 0805, compound 0807, compound 0808, compound 0812, compound 0813, compound 815, compound 816, compound 817, compound 0783, compound 0784, compound 0790, and compound 0792 selectively binds the modified LGIC subunit.

3. The synthetic nucleic acid construct of claim 2, the modified LGIC subunit further comprises an endoplasmic reticulum export sequence, a signal sequence, or a somatic targeting sequence.

4. The synthetic nucleic acid construct of claim 3, wherein the endoplasmic reticulum export sequence comprises the amino acid sequence FCYENEV (SEQ ID NO: 16).

5. The synthetic nucleic acid construct of claim 4, wherein the modified LGIC subunit comprises the amino acid sequence set forth in SEQ ID NO:13.

6. The synthetic nucleic acid construct of claim 2, wherein the signal sequence comprises the amino acid sequence MRRAPSLVLFFLVALCGRGNC (SEQ ID NO: 17).

7. The synthetic nucleic acid construct of claim 6, wherein the modified LGIC subunit comprises the amino acid sequence set forth in SEQ ID NO:14.

8. The synthetic nucleic acid construct of claim 3, wherein the somatic targeting sequence comprises the amino acid sequence
(SEQ ID NO: 18)
QSQPILNTKEMAPQSKPPEELEMSSMPSPVAPLPARTEGVIDMRSMSSID
SFISCATDFPEATRF.

9. The synthetic nucleic acid construct of claim 8, wherein the modified LGIC subunit comprises the amino acid sequence set forth in SEQ ID NO:15.

10. A synthetic nucleic acid construct encoding a modified ligand gated ion channel (LGIC) subunit comprising:
(i) a human alpha 7 nicotinic acetylcholine receptor (α7-nAChR) ligand binding domain (LBD) having an amino acid substitution at one or more amino acid residues selected from the group consisting of residues 77, 79, 115, 131, 139, 141, 175, 210, 216, 217, and 219 as numbered in SEQ ID NO:2;
(ii) a 5HT3 ion pore domain (IPD);
wherein said synthetic nucleic acid construct comprises a nucleic acid sequence having at least 75 percent sequence identity to the sequence set forth in SEQ ID NO:27;
wherein an exogenous LGIC ligand selected from the group consisting of compound 0780, compound 0782, compound 0785, compound 0788, compound 0782, compound 0789, compound 0791, compound 0793, compound 0794, compound 0795, compound 0798, compound 0799, compound 0800, compound 0801, compound 0802, compound 0803, compound 0804, compound 0805, compound 0807, compound 0808, compound 0812, compound 0813, compound 815, compound 816, compound 817, compound 0783, compound 0784, compound 0790, and compound 0792 selectively binds the modified LGIC subunit.

11. A synthetic nucleic acid construct encoding a modified ligand gated ion channel (LGIC) subunit comprising:
(i) a human alpha 7 nicotinic acetylcholine receptor (α7-nAChR) ligand binding domain (LBD) having an amino acid substitution at one or more amino acid residues selected from the group consisting of residues 77, 79, 115, 131, 139, 141, 175, 210, 216, 217, and 219 as numbered in SEQ ID NO:2;
(ii) a GlyR ion pore domain (IPD); and
wherein said synthetic nucleic acid construct comprises a nucleic acid sequence having at least 75 percent sequence identity to the sequence set forth in SEQ ID NO:28;
wherein an exogenous LGIC ligand selected from the group consisting of compound 0780, compound 0782, compound 0785, compound 0788, compound 0782, compound 0789, compound 0791, compound 0793, compound 0794, compound 0795, compound 0798, compound 0799, compound 0800, compound 0801, compound 0802, compound 0803, compound 0804, compound 0805, compound 0807, compound 0808, compound 0812, compound 0813, compound 815, compound 816, compound 817, compound 0783, compound 0784, compound 0790, and compound 0792 selectively binds the modified LGIC subunit.

12. A synthetic nucleic acid construct encoding a modified ligand gated ion channel (LGIC) subunit comprising:
(i) a human alpha 7 nicotinic acetylcholine receptor (α7-nAChR) ligand binding domain (LBD) having an amino acid substitution at one or more amino acid residues selected from the group consisting of residues 77, 79, 115, 131, 139, 141, 175, 210, 216, 217, and 219 as numbered in SEQ ID NO:2;
(ii) a GABA ion pore domain (IPD); and
wherein said synthetic nucleic acid construct comprises a nucleic acid sequence having at least 75 percent sequence identity to the sequence set forth in SEQ ID NO:29;
wherein an exogenous LGIC ligand selected from the group consisting of compound 0780, compound 0782, compound 0785, compound 0788, compound 0782, compound 0789, compound 0791, compound 0793, compound 0794, compound 0795, compound 0798, compound 0799, compound 0800, compound 0801, compound 0802, compound 0803, compound 0804, compound 0805, compound 0807, compound 0808, compound 0812, compound 0813, compound 815, compound 816, compound 817, compound 0783, compound 0784, compound 0790, and compound 0792 selectively binds the modified LGIC subunit.

13. The synthetic nucleic acid construct of claim 11, the modified LGIC subunit further comprises an endoplasmic reticulum export sequence, a signal sequence, or a somatic targeting sequence.

14. The synthetic nucleic acid construct of claim 13, wherein the endoplasmic reticulum export sequence comprises the amino acid sequence FCYENEV (SEQ ID NO: 16).

15. The synthetic nucleic acid construct of claim 14, wherein the modified LGIC subunit comprises the amino acid sequence set forth in SEQ ID NO: 13.

16. The synthetic nucleic acid construct of claim 13, wherein the signal sequence comprises the amino acid sequence MRRAPSLVLFFLVALCGRGNC (SEQ ID NO: 17).

17. The synthetic nucleic acid construct of claim 16, wherein the modified LGIC subunit comprises the amino acid sequence set forth in SEQ ID NO: 14.

18. The synthetic nucleic acid construct of claim 13, wherein the somatic targeting sequence comprises the amino acid sequence
(SEQ ID NO: 18)
QSQPILNTKEMAPQSKPPEELEMSSMPSPVAPLPARTEGVIDMRSMSSID
SFISCATDFPEATRF.

19. The synthetic nucleic acid construct of claim 18, wherein the modified LGIC subunit comprises the amino acid sequence set forth in SEQ ID NO:15.

20. The synthetic nucleic acid construct of claim 2, wherein the α7-nAChR LBD has at least 75% sequence identity to SEQ ID NO:2.

21. The synthetic nucleic acid construct of claim 13, wherein the α7-nAChR LBD has at least 75% sequence identity to SEQ ID NO:2.

22. A viral vector comprising the synthetic nucleic acid construct of claim 2.

23. The viral vector of claim 22, wherein the viral vector is an adeno-associated viral vector.

24. The viral vector of claim 23, wherein the synthetic nucleic acid construct further comprises a synapsin promoter.

25. The viral vector of claim 24, wherein the α7-nAChR LBD comprises an L131G amino acid substitution, a Q139L amino acid substitution, and a Y217F amino acid substitution as numbered in SEQ ID NO:2.
